SEQUENCE LISTING (96 sequences)

SEQ ID NO: 1 - protein, 813 aa, synthetic construct
MGTSLLCWMALCLLGADHADAYPYDVPDYASGGGGSPTTTPAPRPPTPAPTIASQPLSLRPEACRPAAGGAVHTRGLDFACDIYIWAPLAGTCGVLLLSLVITLYCNHRNRRRVCKCPRPVVEGRGSLLTCGDVEENPGPMGWSCIILFLVATATGVHSEIVLTQSPATLSLSPGERATLSCRASQSVRSYLAWYQQKPGQAPRLLIYDASNRATGIPARFSGSGSGTDFTLTISSLEPEDFAVYYCQQRSNWPPTFGCGTKVEIKEPKSSDKTHTSPPSPAPELNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPETATNFSLLKQAGDVEENPGPMETDTLILWVLLLLVPGSTGQLQVQESGPGLVKPSETLSLTCTVSGGSISNSYFSWGWIRQPPGKCLEWIGSFYYGEKTYYNPSLKSRATISIDTSKSQFSLKLSSVTAADTAVYYCPRGPTMIRGVIDSWGQGTLVTVSSEPKSSDKTHTSPPSPAPELNNSSGEMDPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 2 - protein, 813 aa, synthetic construct
MGTSLLCWMALCLLGADHADAYPYDVPDYASGGGGSPTTTPAPRPPTPAPTIASQPLSLRPEACRPAAGGAVHTRGLDFACDIYIWAPLAGTCGVLLLSLVITLYCNHRNRRRVCKCPRPVVEGRGSLLTCGDVEENPGPMGWSCIILFLVATATGVHSEIVLTQSPATLSLSPGERATLSCRASQSVRSYLAWYQQKPGQAPRLLIYDASNRATGIPARFSGSGSGTDFTLTISSLEPEDFAVYYCQQRSNWPPTFGCGTKVEIKEPKSSDKTHTSPPSPAPELNNSSGEMDPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQATNFSLLKQAGDVEENPGPMETDTLILWVLLLLVPGSTGQLQVQESGPGLVKPSETLSLTCTVSGGSISNSYFSWGWIRQPPGKCLEWIGSFYYGEKTYYNPSLKSRATISIDTSKSQFSLKLSSVTAADTAVYYCPRGPTMIRGVIDSWGQGTLVTVSSEPKSSDKTHTSPPSPAPELNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 3 - protein, 867 aa, synthetic construct
MGTSLLCWMALCLLGADHADAYPYDVPDYASGGGGSPTTTPAPRPPTPAPTIASQPLSLRPEACRPAAGGAVHTRGLDFACDIYIWAPLAGTCGVLLLSLVITLYCNHRNRRRVCKCPRPVVEGRGSLLTCGDVEENPGPMGWSCIILFLVATATGVHSEIVLTQSPATLSLSPGERATLSCRASQSVRSYLAWYQQKPGQAPRLLIYDASNRATGIPARFSGSGSGTDFTLTISSLEPEDFAVYYCQQRSNWPPTFGCGTKVEIKEPKSSDKTHTSPPSPAPELNTSKENPFLFALEAVVISVGSMGLIISLLCVYFWLERTMPREKVKTLKAQNSELASTANMLREQVAQLIPTLKNLEDLVTEYHGNFSAWSGVSKGLAESLQPDYSERLCLVSEIPPKGGALGEGPGASPCNQHSPYWAPPCYTLKPETATNFSLLKQAGDVEENPGPMETDTLILWVLLLLVPGSTGQLQVQESGPGLVKPSETLSLTCTVSGGSISNSYFSWGWIRQPPGKCLEWIGSFYYGEKTYYNPSLKSRATISIDTSKSQFSLKLSSVTAADTAVYYCPRGPTMIRGVIDSWGQGTLVTVSSEPKSSDKTHTSPPSPAPELNNSSGEMDPILLTISILSFFSVALLVILACVLWKKRIKPAETDQLEDEKSALQTEIANLLKEKEKLIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 4 - protein, 867 aa, synthetic construct
MGTSLLCWMALCLLGADHADAYPYDVPDYASGGGGSPTTTPAPRPPTPAPTIASQPLSLRPEACRPAAGGAVHTRGLDFACDIYIWAPLAGTCGVLLLSLVITLYCNHRNRRRVCKCPRPVVEGRGSLLTCGDVEENPGPMGWSCIILFLVATATGVHSEIVLTQSPATLSLSPGERATLSCRASQSVRSYLAWYQQKPGQAPRLLIYDASNRATGIPARFSGSGSGTDFTLTISSLEPEDFAVYYCQQRSNWPPTFGCGTKVEIKEPKSSDKTHTSPPSPAPELNNSSGEMDPILLTISILSFFSVALLVILACVLWKKRIKPAETDQLEDEKSALQTEIANLLKEKEKLIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQATNFSLLKQAGDVEENPGPMETDTLILWVLLLLVPGSTGQLQVQESGPGLVKPSETLSLTCTVSGGSISNSYFSWGWIRQPPGKCLEWIGSFYYGEKTYYNPSLKSRATISIDTSKSQFSLKLSSVTAADTAVYYCPRGPTMIRGVIDSWGQGTLVTVSSEPKSSDKTHTSPPSPAPELNTSKENPFLFALEAVVISVGSMGLIISLLCVYFWLERTMPREKVKTLKAQNSELASTANMLREQVAQLIPTLKNLEDLVTEYHGNFSAWSGVSKGLAESLQPDYSERLCLVSEIPPKGGALGEGPGASPCNQHSPYWAPPCYTLKPET

SEQ ID NO: 5 - protein, 1036 aa, synthetic construct
MGTSLLCWMALCLLGADHADAYPYDVPDYASGGGGSPTTTPAPRPPTPAPTIASQPLSLRPEACRPAAGGAVHTRGLDFACDIYIWAPLAGTCGVLLLSLVITLYCNHRNRRRVCKCPRPVVEGRGSLLTCGDVEENPGPMGWSCIILFLVATATGVHSEIVLTQSPATLSLSPGERATLSCRASQSVRSYLAWYQQKPGQAPRLLIYDASNRATGIPARFSGSGSGTDFTLTISSLEPEDFAVYYCQQRSNWPPTFGCGTKVEIKEPKSSDKTHTSPPSPAPELNGIWSEWSEARSWDTESVLPMWVLALIVIFLTIAVLLALRFCGIYGYRLRRKWEEKIPNPSKSHLFQNGSAELWPPGSMSAFTSGSPPHQGPWGSRFPELEGVFPVGFGDSEVSPLTIEDPKHVCDPPSGPDTTPAASDLPTEQPPSPQPGPPAASHTPEKQASSFDFNGPYLGPPHSRSLPDILGQPEPPQEGGSQKSPPPGSLEYLCLPAGGQVQLVPLAQAMGPGQAVEVERRPSQGAAGSPSLESGGGPAPPALGPRVGGQDQKDSPVAIPMSSGDTEDPGVASGYVSSADLVFTPNSGASSVSLVPSLGLPSDQTPSLCPGLASGPPGAPGPVKSGFEGYVELPPIEGRSPRSPRNNPVPPEAKSPVLNPGERPADVSPTSPQPEGLLVLQQVGDYCFLPGLGPGPLSLRSKPSSPGPGPEIKNLDQAFQVKKPPGQAVPQVPVIQLFKALKQQDYLSLPPWEVNKPGEVCATNFSLLKQAGDVEENPGPMETDTLILWVLLLLVPGSTGQLQVQESGPGLVKPSETLSLTCTVSGGSISNSYFSWGWIRQPPGKCLEWIGSFYYGEKTYYNPSLKSRATISIDTSKSQFSLKLSSVTAADTAVYYCPRGPTMIRGVIDSWGQGTLVTVSSEPKSSDKTHTSPPSPAPELNWSSWSEAIEFGSDDGNLGSVYIYVLLIVGTLVCGIVLGFLFKRFLRIQRLFPPVPQIKDKLNDNHEVEDEIIWEEFTPEEGKGYREEVLTVKEIT

SEQ ID NO: 6 - protein, 1036 aa, synthetic construct
MGTSLLCWMALCLLGADHADAYPYDVPDYASGGGGSPTTTPAPRPPTPAPTIASQPLSLRPEACRPAAGGAVHTRGLDFACDIYIWAPLAGTCGVLLLSLVITLYCNHRNRRRVCKCPRPVVEGRGSLLTCGDVEENPGPMGWSCIILFLVATATGVHSEIVLTQSPATLSLSPGERATLSCRASQSVRSYLAWYQQKPGQAPRLLIYDASNRATGIPARFSGSGSGTDFTLTISSLEPEDFAVYYCQQRSNWPPTFGCGTKVEIKEPKSSDKTHTSPPSPAPELNWSSWSEAIEFGSDDGNLGSVYIYVLLIVGTLVCGIVLGFLFKRFLRIQRLFPPVPQIKDKLNDNHEVEDEIIWEEFTPEEGKGYREEVLTVKEITATNFSLLKQAGDVEENPGPMETDTLILWVLLLLVPGSTGQLQVQESGPGLVKPSETLSLTCTVSGGSISNSYFSWGWIRQPPGKCLEWIGSFYYGEKTYYNPSLKSRATISIDTSKSQFSLKLSSVTAADTAVYYCPRGPTMIRGVIDSWGQGTLVTVSSEPKSSDKTHTSPPSPAPELNGIWSEWSEARSWDTESVLPMWVLALIVIFLTIAVLLALRFCGIYGYRLRRKWEEKIPNPSKSHLFQNGSAELWPPGSMSAFTSGSPPHQGPWGSRFPELEGVFPVGFGDSEVSPLTIEDPKHVCDPPSGPDTTPAASDLPTEQPPSPQPGPPAASHTPEKQASSFDFNGPYLGPPHSRSLPDILGQPEPPQEGGSQKSPPPGSLEYLCLPAGGQVQLVPLAQAMGPGQAVEVERRPSQGAAGSPSLESGGGPAPPALGPRVGGQDQKDSPVAIPMSSGDTEDPGVASGYVSSADLVFTPNSGASSVSLVPSLGLPSDQTPSLCPGLASGPPGAPGPVKSGFEGYVELPPIEGRSPRSPRNNPVPPEAKSPVLNPGERPADVSPTSPQPEGLLVLQQVGDYCFLPGLGPGPLSLRSKPSSPGPGPEIKNLDQAFQVKKPPGQAVPQVPVIQLFKALKQQDYLSLPPWEVNKPGEVC

SEQ ID NO: 7 - protein, 1090 aa, synthetic construct
MGTSLLCWMALCLLGADHADAYPYDVPDYASGGGGSPTTTPAPRPPTPAPTIASQPLSLRPEACRPAAGGAVHTRGLDFACDIYIWAPLAGTCGVLLLSLVITLYCNHRNRRRVCKCPRPVVEGRGSLLTCGDVEENPGPMGWSCIILFLVATATGVHSEIVLTQSPATLSLSPGERATLSCRASQSVRSYLAWYQQKPGQAPRLLIYDASNRATGIPARFSGSGSGTDFTLTISSLEPEDFAVYYCQQRSNWPPTFGCGTKVEIKEPKSSDKTHTSPPSPAPELNGIWSEWSEARSWDTESVLPMWVLALIVIFLTIAVLLALRFCGIYEKVKTLKAQNSELASTANMLREQVAQLGYRLRRKWEEKIPNPSKSHLFQNGSAELWPPGSMSAFTSGSPPHQGPWGSRFPELEGVFPVGFGDSEVSPLTIEDPKHVCDPPSGPDTTPAASDLPTEQPPSPQPGPPAASHTPEKQASSFDFNGPYLGPPHSRSLPDILGQPEPPQEGGSQKSPPPGSLEYLCLPAGGQVQLVPLAQAMGPGQAVEVERRPSQGAAGSPSLESGGGPAPPALGPRVGGQDQKDSPVAIPMSSGDTEDPGVASGYVSSADLVFTPNSGASSVSLVPSLGLPSDQTPSLCPGLASGPPGAPGPVKSGFEGYVELPPIEGRSPRSPRNNPVPPEAKSPVLNPGERPADVSPTSPQPEGLLVLQQVGDYCFLPGLGPGPLSLRSKPSSPGPGPEIKNLDQAFQVKKPPGQAVPQVPVIQLFKALKQQDYLSLPPWEVNKPGEVCATNFSLLKQAGDVEENPGPMETDTLILWVLLLLVPGSTGQLQVQESGPGLVKPSETLSLTCTVSGGSISNSYFSWGWIRQPPGKCLEWIGSFYYGEKTYYNPSLKSRATISIDTSKSQFSLKLSSVTAADTAVYYCPRGPTMIRGVIDSWGQGTLVTVSSEPKSSDKTHTSPPSPAPELNWSSWSEAIEFGSDDGNLGSVYIYVLLIVGTLVCGIVLGFLFKRFLRAETDQLEDEKSALQTEIANLLKEKEKLIQRLFPPVPQIKDKLNDNHEVEDEIIWEEFTPEEGKGYREEVLTVKEIT

SEQ ID NO: 8 - protein, 1090 aa, synthetic construct
MGTSLLCWMALCLLGADHADAYPYDVPDYASGGGGSPTTTPAPRPPTPAPTIASQPLSLRPEACRPAAGGAVHTRGLDFACDIYIWAPLAGTCGVLLLSLVITLYCNHRNRRRVCKCPRPVVEGRGSLLTCGDVEENPGPMGWSCIILFLVATATGVHSEIVLTQSPATLSLSPGERATLSCRASQSVRSYLAWYQQKPGQAPRLLIYDASNRATGIPARFSGSGSGTDFTLTISSLEPEDFAVYYCQQRSNWPPTFGCGTKVEIKEPKSSDKTHTSPPSPAPELNWSSWSEAIEFGSDDGNLGSVYIYVLLIVGTLVCGIVLGFLFKRFLRAETDQLEDEKSALQTEIANLLKEKEKLIQRLFPPVPQIKDKLNDNHEVEDEIIWEEFTPEEGKGYREEVLTVKEITATNFSLLKQAGDVEENPGPMETDTLILWVLLLLVPGSTGQLQVQESGPGLVKPSETLSLTCTVSGGSISNSYFSWGWIRQPPGKCLEWIGSFYYGEKTYYNPSLKSRATISIDTSKSQFSLKLSSVTAADTAVYYCPRGPTMIRGVIDSWGQGTLVTVSSEPKSSDKTHTSPPSPAPELNGIWSEWSEARSWDTESVLPMWVLALIVIFLTIAVLLALRFCGIYEKVKTLKAQNSELASTANMLREQVAQLGYRLRRKWEEKIPNPSKSHLFQNGSAELWPPGSMSAFTSGSPPHQGPWGSRFPELEGVFPVGFGDSEVSPLTIEDPKHVCDPPSGPDTTPAASDLPTEQPPSPQPGPPAASHTPEKQASSFDFNGPYLGPPHSRSLPDILGQPEPPQEGGSQKSPPPGSLEYLCLPAGGQVQLVPLAQAMGPGQAVEVERRPSQGAAGSPSLESGGGPAPPALGPRVGGQDQKDSPVAIPMSSGDTEDPGVASGYVSSADLVFTPNSGASSVSLVPSLGLPSDQTPSLCPGLASGPPGAPGPVKSGFEGYVELPPIEGRSPRSPRNNPVPPEAKSPVLNPGERPADVSPTSPQPEGLLVLQQVGDYCFLPGLGPGPLSLRSKPSSPGPGPEIKNLDQAFQVKKPPGQAVPQVPVIQLFKALKQQDYLSLPPWEVNKPGEVC

SEQ ID NO: 9 - protein, 542 aa, synthetic construct
MGTSLLCWMALCLLGADHADAYPYDVPDYASGGGGSPTTTPAPRPPTPAPTIASQPLSLRPEACRPAAGGAVHTRGLDFACDIYIWAPLAGTCGVLLLSLVITLYCNHRNRRRVCKCPRPVVEGRGSLLTCGDVEENPGPMGWSCIILFLVATATGVHSEIVLTQSPATLSLSPGERATLSCRASQSVRSYLAWYQQKPGQAPRLLIYDASNRATGIPARFSGSGSGTDFTLTISSLEPEDFAVYYCQQRSNWPPTFGCGTKVEIKEPKSSDKTHTSPPSPAPELSPLFPGPSKPFWVLVVVGGVLACYSLLVTVAFIIFWVRSATNFSLLKQAGDVEENPGPMETDTLILWVLLLLVPGSTGQLQVQESGPGLVKPSETLSLTCTVSGGSISNSYFSWGWIRQPPGKCLEWIGSFYYGEKTYYNPSLKSRATISIDTSKSQFSLKLSSVTAADTAVYYCPRGPTMIRGVIDSWGQGTLVTVSSEPKSSDKTHTSPPSPAPELSPLFPGPSKPFWVLVVVGGVLACYSLLVTVAFIIFWVRS

SEQ ID NO: 10 - protein, 536 aa, synthetic construct
MGTSLLCWMALCLLGADHADAYPYDVPDYASGGGGSPTTTPAPRPPTPAPTIASQPLSLRPEACRPAAGGAVHTRGLDFACDIYIWAPLAGTCGVLLLSLVITLYCNHRNRRRVCKCPRPVVEGRGSLLTCGDVEENPGPMGWSCIILFLVATATGVHSEIVLTQSPATLSLSPGERATLSCRASQSVRSYLAWYQQKPGQAPRLLIYDASNRATGIPARFSGSGSGTDFTLTISSLEPEDFAVYYCQQRSNWPPTFGCGTKVEIKEPKSSDKTHTSPPSPAPELNNSSGEMDPILLTISILSFFSVALLVILACVLWKKATNFSLLKQAGDVEENPGPMETDTLILWVLLLLVPGSTGQLQVQESGPGLVKPSETLSLTCTVSGGSISNSYFSWGWIRQPPGKCLEWIGSFYYGEKTYYNPSLKSRATISIDTSKSQFSLKLSSVTAADTAVYYCPRGPTMIRGVIDSWGQGTLVTVSSEPKSSDKTHTSPPSPAPELNTSKENPFLFALEAVVISVGSMGLIISLLCVYFWLER

SEQ ID NO: 11 - protein, 1043 aa, synthetic construct
MGTSLLCWMALCLLGADHADAYPYDVPDYASGGGGSPTTTPAPRPPTPAPTIASQPLSLRPEACRPAAGGAVHTRGLDFACDIYIWAPLAGTCGVLLLSLVITLYCNHRNRRRVCKCPRPVVEGRGSLLTCGDVEENPGPMGWSCIILFLVATATGVHSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEPKSCDKTHTCPPCPEPKSSDKTHTSPPSPAPELNGIWSEWSEARSWDTESVLPMWVLALIVIFLTIAVLLALRFCGIYGYRLRRKWEEKIPNPSKSHLFQNGSAELWPPGSMSAFTSGSPPHQGPWGSRFPELEGVFPVGFGDSEVSPLTIEDPKHVCDPPSGPDTTPAASDLPTEQPPSPQPGPPAASHTPEKQASSFDFNGPYLGPPHSRSLPDILGQPEPPQEGGSQKSPPPGSLEYLCLPAGGQVQLVPLAQAMGPGQAVEVERRPSQGAAGSPSLESGGGPAPPALGPRVGGQDQKDSPVAIPMSSGDTEDPGVASGYVSSADLVFTPNSGASSVSLVPSLGLPSDQTPSLCPGLASGPPGAPGPVKSGFEGYVELPPIEGRSPRSPRNNPVPPEAKSPVLNPGERPADVSPTSPQPEGLLVLQQVGDYCFLPGLGPGPLSLRSKPSSPGPGPEIKNLDQAFQVKKPPGQAVPQVPVIQLFKALKQQDYLSLPPWEVNKPGEVCATNFSLLKQAGDVEENPGPMETDTLILWVLLLLVPGSTGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPEPKSSDKTHTSPPSPAPELNWSSWSEAIEFGSDDGNLGSVYIYVLLIVGTLVCGIVLGFLFKRFLRIQRLFPPVPQIKDKLNDNHEVEDEIIWEEFTPEEGKGYREEVLTVKEIT

SEQ ID NO: 12 - protein, 1032 aa, synthetic construct
MGTSLLCWMALCLLGADHADAYPYDVPDYASGGGGSPTTTPAPRPPTPAPTIASQPLSLRPEACRPAAGGAVHTRGLDFACDIYIWAPLAGTCGVLLLSLVITLYCNHRNRRRVCKCPRPVVEGRGSLLTCGDVEENPGPMGWSCIILFLVATATGVHSDIQMTQSPSSLSASVGDRVTITCKASQNVGINVAWYQQKPGKAPKSLISSASYRYSGVPSRFSGSGSGTDFTLTISSLQPEDFATYFCQQYDTYPFTFGCGTKVEIKEPKSSDKTHTSPPSPAPELNGIWSEWSEARSWDTESVLPMWVLALIVIFLTIAVLLALRFCGIYGYRLRRKWEEKIPNPSKSHLFQNGSAELWPPGSMSAFTSGSPPHQGPWGSRFPELEGVFPVGFGDSEVSPLTIEDPKHVCDPPSGPDTTPAASDLPTEQPPSPQPGPPAASHTPEKQASSFDFNGPYLGPPHSRSLPDILGQPEPPQEGGSQKSPPPGSLEYLCLPAGGQVQLVPLAQAMGPGQAVEVERRPSQGAAGSPSLESGGGPAPPALGPRVGGQDQKDSPVAIPMSSGDTEDPGVASGYVSSADLVFTPNSGASSVSLVPSLGLPSDQTPSLCPGLASGPPGAPGPVKSGFEGYVELPPIEGRSPRSPRNNPVPPEAKSPVLNPGERPADVSPTSPQPEGLLVLQQVGDYCFLPGLGPGPLSLRSKPSSPGPGPEIKNLDQAFQVKKPPGQAVPQVPVIQLFKALKQQDYLSLPPWEVNKPGEVCATNFSLLKQAGDVEENPGPMETDTLILWVLLLLVPGSTGQVQLVQSGAEEVKKPGSSVKVSCKASGYTFTTYYLHWVRQAPGQCLEWMGWIYPGNVHAQYNEKFKGRVTITADKSTSTAYMELSSLRSEDTAVYYCARSWEGFPYWGQGTTVTVSSEPKSSDKTHTSPPSPAPELNWSSWSEAIEFGSDDGNLGSVYIYVLLIVGTLVCGIVLGFLFKRFLRIQRLFPPVPQIKDKLNDNHEVEDEIIWEEFTPEEGKGYREEVLTVKEIT

SEQ ID NO: 13 - DNA, 2439 bp, synthetic construct
  source 1..2439: mol_type = other DNA
atgggcaccagcctgctgtgctggatggccctgtgcctgctgggcgccgaccacgccgatgcctacccctacgacgtgcccgactacgccagcggagggggaggcagccccaccaccaccccagcccccagacctcccaccccagcccccaccatcgccagccagcctctgagcctgagacccgaggcctgccgcccagcggccggcggcgccgtgcacaccagaggcctggatttcgcctgcgatatctacatctgggccccactcgccggcacctgtggcgtgctgctgctgagcctggtgatcaccctgtactgcaaccaccgcaaccgcaggcgcgtgtgcaagtgccccaggcccgtcgtggaaggcagaggcagcctgcttacatgtggcgacgtggaagagaaccccggacctatgggctggagctgcatcatcctgttcctggtggccaccgccaccggcgtgcacagcgagatcgtgctgacacagtctcccgccacactgtcactgtctccaggcgaaagagccacactgagctgtagagcctctcagagcgtgcggtcttacctggcctggtatcagcagaagcctggacaggctccccggctgctgatctacgatgccagcaatagagccacaggcatccccgccagattttctggctctggcagcggcaccgatttcaccctgaccataagcagcctggaacctgaggacttcgccgtgtactactgccagcagagaagcaactggcctcctacctttggctgcggcaccaaggtggaaatcaaggagcccaagagcagcgacaagacccacaccagcccccccagccccgcccccgagctgaacaccagcaaggaaaacccctttctgttcgccctggaggccgtggttatcagcgtgggctctatgggcctgatcatcagcctgctgtgcgtgtacttctggctggaacggaccatgcctagaatccccacactgaagaaccttgaggacctggtcaccgagtaccacggcaacttttctgcttggagcggcgtgtccaaaggcctggccgaatctctccagcctgactacagcgagagactgtgcctggtgtctgagatccctcctaaaggcggagccctcggagaaggacctggcgcctctccatgtaatcagcacagcccttactgggctcctccttgctacaccctgaagcctgagacagccaccaatttcagcctgctgaaacaggctggcgacgtggaagagaaccctggacctatggagaccgacaccctgatcctgtgggtgctgctgctgctggtgcccggcagcaccggccagctccaggtgcaagagtctggacctggcctggtcaagcctagcgagacactgagcctgacctgtacagtgtctggcggcagcatcagcaacagctactttagctggggctggatcagacagcctcctggcaagtgcctggaatggatcggcagcttctactacggcgagaaaacctactacaaccccagcctgaagtcccgggccaccatcagcatcgataccagcaagagccagttctccctgaagctgagcagcgtgacagccgccgatacagccgtgtactactgtcctagaggccccaccatgatccggggcgtgatagattcttggggccagggcacactggtcaccgtttcttctgagccgaaatcctccgataaaacgcatacatccccgccatcaccagcgccagaactgaacaacagctccggcgaaatggaccctatcctgctgacaatcagcatcctgagctttttcagcgtggccctgctggtcatcctggcctgtgtgctgtggaagaagcggatcaagcccatcgtgtggcccagcctgcctgaccacaagaaaaccctggaacacctgtgcaagaagccccggaagaacctgaacgtgtccttcaatcccgagagcttcctggactgccagatccacagagtggacgacatccaggccagggacgaagtggaaggcttcctccaggacacattccctcagcagctggaagagagcgagaagcagagactcggcggagatgtgcagagccctaattgccctagcgaggacgtggtcatcacccctgagagcttcggcagagatagcagcctgacatgtctggccggcaatgtgtccgcctgtgatgcccctatcctgagcagtagcagaagcctggattgcagagagagcggcaagaacggccctcacgtgtaccaggatctgctgctgtctctgggcaccaccaatagcacactgcctccacctttcagcctgcaaagcggcatcctgacactgaaccctgtggctcagggacagccaatcctgacaagcctgggcagcaatcaagaagaggcctacgtcaccatgagcagcttctaccagaaccag

SEQ ID NO: 14 - DNA, 2439 bp, synthetic construct
  source 1..2439: mol_type = other DNA
atgggcaccagcctgctgtgctggatggccctgtgcctgctgggcgccgaccacgccgatgcctacccctacgacgtgcccgactacgccagcggagggggaggcagccccaccaccaccccagcccccagacctcccaccccagcccccaccatcgccagccagcctctgagcctgagacccgaggcctgccgcccagcggccggcggcgccgtgcacaccagaggcctggatttcgcctgcgatatctacatctgggccccactcgccggcacctgtggcgtgctgctgctgagcctggtgatcaccctgtactgcaaccaccgcaaccgcaggcgcgtgtgcaagtgccccaggcccgtcgtggaaggcagaggcagcctgcttacatgtggcgacgtggaagagaaccccggacctatgggctggagctgcatcatcctgttcctggtggccaccgccaccggcgtgcacagcgagatcgtgctgacacagtctcccgccacactgtcactgtctccaggcgaaagagccacactgagctgtagagcctctcagagcgtgcggtcttacctggcctggtatcagcagaagcctggacaggctccccggctgctgatctacgatgccagcaatagagccacaggcatccccgccagattttctggctctggcagcggcaccgatttcaccctgaccataagcagcctggaacctgaggacttcgccgtgtactactgccagcagagaagcaactggcctcctacctttggctgcggcaccaaggtggaaatcaaggagcccaagagcagcgacaagacccacaccagcccccccagccccgcccccgagctgaacaacagctccggcgaaatggaccctatcctgctgacaatcagcatcctgagctttttcagcgtggccctgctggtcatcctggcctgtgtgctgtggaagaagcggatcaagcccatcgtgtggcccagcctgcctgaccacaagaaaaccctggaacacctgtgcaagaagccccggaagaacctgaacgtgtccttcaatcccgagagcttcctggactgccagatccacagagtggacgacatccaggccagggacgaagtggaaggcttcctccaggacacattccctcagcagctggaagagagcgagaagcagagactcggcggagatgtgcagagccctaattgccctagcgaggacgtggtcatcacccctgagagcttcggcagagatagcagcctgacatgtctggccggcaatgtgtccgcctgtgatgcccctatcctgagcagtagcagaagcctggattgcagagagagcggcaagaacggccctcacgtgtaccaggatctgctgctgtctctgggcaccaccaatagcacactgcctccacctttcagcctgcaaagcggcatcctgacactgaaccctgtggctcagggacagccaatcctgacaagcctgggcagcaatcaagaagaggcctacgtcaccatgagcagcttctaccagaaccaggccaccaatttcagcctgctgaaacaggctggcgacgtggaagagaaccctggacctatggagaccgacaccctgatcctgtgggtgctgctgctgctggtgcccggcagcaccggccagctccaggtgcaagagtctggacctggcctggtcaagcctagcgagacactgagcctgacctgtacagtgtctggcggcagcatcagcaacagctactttagctggggctggatcagacagcctcctggcaagtgcctggaatggatcggcagcttctactacggcgagaaaacctactacaaccccagcctgaagtcccgggccaccatcagcatcgataccagcaagagccagttctccctgaagctgagcagcgtgacagccgccgatacagccgtgtactactgtcctagaggccccaccatgatccggggcgtgatagattcttggggccagggcacactggtcaccgtttcttctgagccgaaatcctccgataaaacgcatacatccccgccatcaccagcgccagaactgaacaccagcaaggaaaacccctttctgttcgccctggaggccgtggttatcagcgtgggctctatgggcctgatcatcagcctgctgtgcgtgtacttctggctggaacggaccatgcctagaatccccacactgaagaaccttgaggacctggtcaccgagtaccacggcaacttttctgcttggagcggcgtgtccaaaggcctggccgaatctctccagcctgactacagcgagagactgtgcctggtgtctgagatccctcctaaaggcggagccctcggagaaggacctggcgcctctccatgtaatcagcacagcccttactgggctcctccttgctacaccctgaagcctgagaca

SEQ ID NO: 15 - DNA, 2601 bp, synthetic construct
  source 1..2601: mol_type = other DNA
atgggcaccagcctgctgtgctggatggccctgtgcctgctgggcgccgaccacgccgatgcctacccctacgacgtgcccgactacgccagcggagggggaggcagccccaccaccaccccagcccccagacctcccaccccagcccccaccatcgccagccagcctctgagcctgagacccgaggcctgccgcccagcggccggcggcgccgtgcacaccagaggcctggatttcgcctgcgatatctacatctgggccccactcgccggcacctgtggcgtgctgctgctgagcctggtgatcaccctgtactgcaaccaccgcaaccgcaggcgcgtgtgcaagtgccccaggcccgtcgtggaaggcagaggcagcctgcttacatgtggcgacgtggaagagaaccccggacctatgggctggagctgcatcatcctgttcctggtggccaccgccaccggcgtgcacagcgagatcgtgctgacacagtctcccgccacactgtcactgtctccaggcgaaagagccacactgagctgtagagcctctcagagcgtgcggtcttacctggcctggtatcagcagaagcctggacaggctccccggctgctgatctacgatgccagcaatagagccacaggcatccccgccagattttctggctctggcagcggcaccgatttcaccctgaccataagcagcctggaacctgaggacttcgccgtgtactactgccagcagagaagcaactggcctcctacctttggctgcggcaccaaggtggaaatcaaggagcccaagagcagcgacaagacccacaccagcccccccagccccgcccccgagctgaacaccagcaaggaaaacccctttctgttcgccctggaggccgtggttatcagcgtgggctctatgggcctgatcatcagcctgctgtgcgtgtacttctggctggaacggaccatgcctagagagaaagtcaagaccctgaaggcccagaacagcgagctggcctctaccgccaacatgctgagagaacaggtggcccaactgatccccacactgaagaaccttgaggacctggtcaccgagtaccacggcaacttttctgcttggagcggcgtgtccaaaggcctggccgaatctctccagcctgactacagcgagagactgtgcctggtgtctgagatccctcctaaaggcggagccctcggagaaggacctggcgcctctccatgtaatcagcacagcccttactgggctcctccttgctacaccctgaagcctgagacagccaccaatttcagcctgctgaaacaggctggcgacgtggaagagaaccctggacctatggagaccgacaccctgatcctgtgggtgctgctgctgctggtgcccggcagcaccggccagctccaggtgcaagagtctggacctggcctggtcaagcctagcgagacactgagcctgacctgtacagtgtctggcggcagcatcagcaacagctactttagctggggctggatcagacagcctcctggcaagtgcctggaatggatcggcagcttctactacggcgagaaaacctactacaaccccagcctgaagtcccgggccaccatcagcatcgataccagcaagagccagttctccctgaagctgagcagcgtgacagccgccgatacagccgtgtactactgtcctagaggccccaccatgatccggggcgtgatagattcttggggccagggcacactggtcaccgtttcttctgagccgaaatcctccgataaaacgcatacatccccgccatcaccagcgccagaactgaacaacagctccggcgaaatggaccctatcctgctgacaatcagcatcctgagctttttcagcgtggccctgctggtcatcctggcctgtgtgctgtggaagaagcggatcaagcccgccgagacagaccagctggaagatgagaagtctgccctccagaccgagatcgccaacctgctgaaagagaaagagaagctgatcgtgtggcccagcctgcctgaccacaagaaaaccctggaacacctgtgcaagaagccccggaagaacctgaacgtgtccttcaatcccgagagcttcctggactgccagatccacagagtggacgacatccaggccagggacgaagtggaaggcttcctccaggacacattccctcagcagctggaagagagcgagaagcagagactcggcggagatgtgcagagccctaattgccctagcgaggacgtggtcatcacccctgagagcttcggcagagatagcagcctgacatgtctggccggcaatgtgtccgcctgtgatgcccctatcctgagcagtagcagaagcctggattgcagagagagcggcaagaacggccctcacgtgtaccaggatctgctgctgtctctgggcaccaccaatagcacactgcctccacctttcagcctgcaaagcggcatcctgacactgaaccctgtggctcagggacagccaatcctgacaagcctgggcagcaatcaagaagaggcctacgtcaccatgagcagcttctaccagaaccag

SEQ ID NO: 16 - DNA, 2601 bp, synthetic construct
  source 1..2601: mol_type = other DNA
atgggcaccagcctgctgtgctggatggccctgtgcctgctgggcgccgaccacgccgatgcctacccctacgacgtgcccgactacgccagcggagggggaggcagccccaccaccaccccagcccccagacctcccaccccagcccccaccatcgccagccagcctctgagcctgagacccgaggcctgccgcccagcggccggcggcgccgtgcacaccagaggcctggatttcgcctgcgatatctacatctgggccccactcgccggcacctgtggcgtgctgctgctgagcctggtgatcaccctgtactgcaaccaccgcaaccgcaggcgcgtgtgcaagtgccccaggcccgtcgtggaaggcagaggcagcctgcttacatgtggcgacgtggaagagaaccccggacctatgggctggagctgcatcatcctgttcctggtggccaccgccaccggcgtgcacagcgagatcgtgctgacacagtctcccgccacactgtcactgtctccaggcgaaagagccacactgagctgtagagcctctcagagcgtgcggtcttacctggcctggtatcagcagaagcctggacaggctccccggctgctgatctacgatgccagcaatagagccacaggcatccccgccagattttctggctctggcagcggcaccgatttcaccctgaccataagcagcctggaacctgaggacttcgccgtgtactactgccagcagagaagcaactggcctcctacctttggctgcggcaccaaggtggaaatcaaggagcccaagagcagcgacaagacccacaccagcccccccagccccgcccccgagctgaacaacagctccggcgaaatggaccctatcctgctgacaatcagcatcctgagctttttcagcgtggccctgctggtcatcctggcctgtgtgctgtggaagaagcggatcaagcccgccgagacagaccagctggaagatgagaagtctgccctccagaccgagatcgccaacctgctgaaagagaaagagaagctgatcgtgtggcccagcctgcctgaccacaagaaaaccctggaacacctgtgcaagaagccccggaagaacctgaacgtgtccttcaatcccgagagcttcctggactgccagatccacagagtggacgacatccaggccagggacgaagtggaaggcttcctccaggacacattccctcagcagctggaagagagcgagaagcagagactcggcggagatgtgcagagccctaattgccctagcgaggacgtggtcatcacccctgagagcttcggcagagatagcagcctgacatgtctggccggcaatgtgtccgcctgtgatgcccctatcctgagcagtagcagaagcctggattgcagagagagcggcaagaacggccctcacgtgtaccaggatctgctgctgtctctgggcaccaccaatagcacactgcctccacctttcagcctgcaaagcggcatcctgacactgaaccctgtggctcagggacagccaatcctgacaagcctgggcagcaatcaagaagaggcctacgtcaccatgagcagcttctaccagaaccaggccaccaatttcagcctgctgaaacaggctggcgacgtggaagagaaccctggacctatggagaccgacaccctgatcctgtgggtgctgctgctgctggtgcccggcagcaccggccagctccaggtgcaagagtctggacctggcctggtcaagcctagcgagacactgagcctgacctgtacagtgtctggcggcagcatcagcaacagctactttagctggggctggatcagacagcctcctggcaagtgcctggaatggatcggcagcttctactacggcgagaaaacctactacaaccccagcctgaagtcccgggccaccatcagcatcgataccagcaagagccagttctccctgaagctgagcagcgtgacagccgccgatacagccgtgtactactgtcctagaggccccaccatgatccggggcgtgatagattcttggggccagggcacactggtcaccgtttcttctgagccgaaatcctccgataaaacgcatacatccccgccatcaccagcgccagaactgaacaccagcaaggaaaacccctttctgttcgccctggaggccgtggttatcagcgtgggctctatgggcctgatcatcagcctgctgtgcgtgtacttctggctggaacggaccatgcctagagagaaagtcaagaccctgaaggcccagaacagcgagctggcctctaccgccaacatgctgagagaacaggtggcccaactgatccccacactgaagaaccttgaggacctggtcaccgagtaccacggcaacttttctgcttggagcggcgtgtccaaaggcctggccgaatctctccagcctgactacagcgagagactgtgcctggtgtctgagatccctcctaaaggcggagccctcggagaaggacctggcgcctctccatgtaatcagcacagcccttactgggctcctccttgctacaccctgaagcctgagaca

SEQ ID NO: 17 - DNA, 3108 bp, synthetic construct
  source 1..3108: mol_type = other DNA
atgggcaccagcctgctgtgctggatggccctgtgcctgctgggcgccgaccacgccgatgcctacccctacgacgtgcccgactacgccagcggagggggaggcagccccaccaccaccccagcccccagacctcccaccccagcccccaccatcgccagccagcctctgagcctgagacccgaggcctgccgcccagcggccggcggcgccgtgcacaccagaggcctggatttcgcctgcgatatctacatctgggccccactcgccggcacctgtggcgtgctgctgctgagcctggtgatcaccctgtactgcaaccaccgcaaccgcaggcgcgtgtgcaagtgccccaggcccgtcgtggaaggcagaggcagcctgcttacatgtggcgacgtggaagagaaccccggacctatgggctggagctgcatcatcctgttcctggtggccaccgccaccggcgtgcacagcgagatcgtgctgacacagtctcccgccacactgtcactgtctccaggcgaaagagccacactgagctgtagagcctctcagagcgtgcggtcttacctggcctggtatcagcagaagcctggacaggctccccggctgctgatctacgatgccagcaatagagccacaggcatccccgccagattttctggctctggcagcggcaccgatttcaccctgaccataagcagcctggaacctgaggacttcgccgtgtactactgccagcagagaagcaactggcctcctacctttggctgcggcaccaaggtggaaatcaaggagcccaagagcagcgacaagacccacaccagcccccccagccccgcccccgagctgaacggcatttggagcgagtggagcgaagccagaagctgggataccgagagcgtgctgcctatgtgggtgctcgccctgatcgtgatcttcctgacaatcgccgtgctgctggccctgagattctgtggcatctacggctacagactgcggcggaagtgggaagagaagatccccaatcctagcaagagccacctgttccagaatggctctgccgagctttggcctcctggcagcatgagcgcctttacatctggcagccctccacatcaaggcccctggggaagcagattccctgaactggaaggcgtgttccctgtcggcttcggcgatagcgaagtgtcccctctgaccatcgaggaccctaagcacgtgtgcgatcctccaagcggccctgatacaacaccagccgcaagcgatctgcctaccgagcaacctcctagtcctcagcctggacctcctgccgcttctcacacacctgagaaacaggccagctccttcgacttcaacggcccctatctgggccctcctcatagcagaagcctgcctgacattctgggccagcctgagcctcctcaagaaggcggatctcagaagtctcctccacctggcagcctggaatacctgtgtttgcctgctggtggccaggtgcagctggttcctcttgctcaagctatgggacctggacaggccgtggaagtggaaagaaggccatctcaaggcgccgctggctctccatctcttgaatctggcggaggacctgctcctcctgctctgggacctagagttggaggccaggaccagaaagacagccccgtggccattcctatgagcagcggagataccgaagatcccggcgtggcctctggctatgtgtcatctgccgacctggtgttcacccctaatagcggcgctagctccgtgtctctggtgcctagtcttggcctgcctagcgatcagaccccttctctgtgtcctggactggcttctggaccacctggtgctccaggacctgtgaagtctggctttgagggctacgtggaactgcctcctatcgagggcagaagccctagaagccccagaaacaaccctgtgcctcctgaggccaagtctcccgttctgaaccctggcgaaagacccgccgatgtgtctcctacatctcctcagccagagggactgctggtgctgcaacaagtgggcgactactgcttcctgcctggacttggaccaggacctctgagcctgagaagcaagcctagctctcctggacctgggcctgagatcaagaatctggaccaggccttccaagtgaagaagcctccaggccaggctgtgcctcaggtgccagtgatccagctgttcaaggccctgaagcagcaggactacctgagcctgccaccttgggaagtgaacaagccaggcgaagtgtgcgccaccaatttcagcctgctgaaacaggctggcgacgtggaagagaaccctggacctatggagaccgacaccctgatcctgtgggtgctgctgctgctggtgcccggcagcaccggccagctccaggtgcaagagtctggacctggcctggtcaagcctagcgagacactgagcctgacctgtacagtgtctggcggcagcatcagcaacagctactttagctggggctggatcagacagcctcctggcaagtgcctggaatggatcggcagcttctactacggcgagaaaacctactacaaccccagcctgaagtcccgggccaccatcagcatcgataccagcaagagccagttctccctgaagctgagcagcgtgacagccgccgatacagccgtgtactactgtcctagaggccccaccatgatccggggcgtgatagattcttggggccagggcacactggtcaccgtttcttctgagccgaaatcctccgataaaacgcatacatccccgccatcaccagcgccagaactgaactggtcctcttggagcgaggccatcgagttcggcagcgacgatggcaatctgggcagcgtgtacatctacgtgctgctgatcgtgggcaccctcgtgtgtggaatcgtgctgggcttcctgttcaagcggttcctgagaatccagcggctgttccctccagtgcctcagatcaaggacaagctgaacgacaaccacgaggtcgaggacgagatcatctgggaagagttcacccctgaggaaggcaagggctacagagaagaggtgctgaccgtgaaagagatcacc

SEQ ID NO: 18 - DNA, 3108 bp, synthetic construct
  source 1..3108: mol_type = other DNA
atgggcaccagcctgctgtgctggatggccctgtgcctgctgggcgccgaccacgccgatgcctacccctacgacgtgcccgactacgccagcggagggggaggcagccccaccaccaccccagcccccagacctcccaccccagcccccaccatcgccagccagcctctgagcctgagacccgaggcctgccgcccagcggccggcggcgccgtgcacaccagaggcctggatttcgcctgcgatatctacatctgggccccactcgccggcacctgtggcgtgctgctgctgagcctggtgatcaccctgtactgcaaccaccgcaaccgcaggcgcgtgtgcaagtgccccaggcccgtcgtggaaggcagaggcagcctgcttacatgtggcgacgtggaagagaaccccggacctatgggctggagctgcatcatcctgttcctggtggccaccgccaccggcgtgcacagcgagatcgtgctgacacagtctcccgccacactgtcactgtctccaggcgaaagagccacactgagctgtagagcctctcagagcgtgcggtcttacctggcctggtatcagcagaagcctggacaggctccccggctgctgatctacgatgccagcaatagagccacaggcatccccgccagattttctggctctggcagcggcaccgatttcaccctgaccataagcagcctggaacctgaggacttcgccgtgtactactgccagcagagaagcaactggcctcctacctttggctgcggcaccaaggtggaaatcaaggagcccaagagcagcgacaagacccacaccagcccccccagccccgcccccgagctgaactggtcctcttggagcgaggccatcgagttcggcagcgacgatggcaatctgggcagcgtgtacatctacgtgctgctgatcgtgggcaccctcgtgtgtggaatcgtgctgggcttcctgttcaagcggttcctgagaatccagcggctgttccctccagtgcctcagatcaaggacaagctgaacgacaaccacgaggtcgaggacgagatcatctgggaagagttcacccctgaggaaggcaagggctacagagaagaggtgctgaccgtgaaagagatcaccgccaccaatttcagcctgctgaaacaggctggcgacgtggaagagaaccctggacctatggagaccgacaccctgatcctgtgggtgctgctgctgctggtgcccggcagcaccggccagctccaggtgcaagagtctggacctggcctggtcaagcctagcgagacactgagcctgacctgtacagtgtctggcggcagcatcagcaacagctactttagctggggctggatcagacagcctcctggcaagtgcctggaatggatcggcagcttctactacggcgagaaaacctactacaaccccagcctgaagtcccgggccaccatcagcatcgataccagcaagagccagttctccctgaagctgagcagcgtgacagccgccgatacagccgtgtactactgtcctagaggccccaccatgatccggggcgtgatagattcttggggccagggcacactggtcaccgtttcttctgagccgaaatcctccgataaaacgcatacatccccgccatcaccagcgccagaactgaacggcatttggagcgagtggagcgaagccagaagctgggataccgagagcgtgctgcctatgtgggtgctcgccctgatcgtgatcttcctgacaatcgccgtgctgctggccctgagattctgtggcatctacggctacagactgcggcggaagtgggaagagaagatccccaatcctagcaagagccacctgttccagaatggctctgccgagctttggcctcctggcagcatgagcgcctttacatctggcagccctccacatcaaggcccctggggaagcagattccctgaactggaaggcgtgttccctgtcggcttcggcgatagcgaagtgtcccctctgaccatcgaggaccctaagcacgtgtgcgatcctccaagcggccctgatacaacaccagccgcaagcgatctgcctaccgagcaacctcctagtcctcagcctggacctcctgccgcttctcacacacctgagaaacaggccagctccttcgacttcaacggcccctatctgggccctcctcatagcagaagcctgcctgacattctgggccagcctgagcctcctcaagaaggcggatctcagaagtctcctccacctggcagcctggaatacctgtgtttgcctgctggtggccaggtgcagctggttcctcttgctcaagctatgggacctggacaggccgtggaagtggaaagaaggccatctcaaggcgccgctggctctccatctcttgaatctggcggaggacctgctcctcctgctctgggacctagagttggaggccaggaccagaaagacagccccgtggccattcctatgagcagcggagataccgaagatcccggcgtggcctctggctatgtgtcatctgccgacctggtgttcacccctaatagcggcgctagctccgtgtctctggtgcctagtcttggcctgcctagcgatcagaccccttctctgtgtcctggactggcttctggaccacctggtgctccaggacctgtgaagtctggctttgagggctacgtggaactgcctcctatcgagggcagaagccctagaagccccagaaacaaccctgtgcctcctgaggccaagtctcccgttctgaaccctggcgaaagacccgccgatgtgtctcctacatctcctcagccagagggactgctggtgctgcaacaagtgggcgactactgcttcctgcctggacttggaccaggacctctgagcctgagaagcaagcctagctctcctggacctgggcctgagatcaagaatctggaccaggccttccaagtgaagaagcctccaggccaggctgtgcctcaggtgccagtgatccagctgttcaaggccctgaagcagcaggactacctgagcctgccaccttgggaagtgaacaagccaggcgaagtgtgc

SEQ ID NO: 19 - DNA, 3270 bp, synthetic construct
  source 1..3270: mol_type = other DNA
atgggcaccagcctgctgtgctggatggccctgtgcctgctgggcgccgaccacgccgatgcctacccctacgacgtgcccgactacgccagcggagggggaggcagccccaccaccaccccagcccccagacctcccaccccagcccccaccatcgccagccagcctctgagcctgagacccgaggcctgccgcccagcggccggcggcgccgtgcacaccagaggcctggatttcgcctgcgatatctacatctgggccccactcgccggcacctgtggcgtgctgctgctgagcctggtgatcaccctgtactgcaaccaccgcaaccgcaggcgcgtgtgcaagtgccccaggcccgtcgtggaaggcagaggcagcctgcttacatgtggcgacgtggaagagaaccccggacctatgggctggagctgcatcatcctgttcctggtggccaccgccaccggcgtgcacagcgagatcgtgctgacacagtctcccgccacactgtcactgtctccaggcgaaagagccacactgagctgtagagcctctcagagcgtgcggtcttacctggcctggtatcagcagaagcctggacaggctccccggctgctgatctacgatgccagcaatagagccacaggcatccccgccagattttctggctctggcagcggcaccgatttcaccctgaccataagcagcctggaacctgaggacttcgccgtgtactactgccagcagagaagcaactggcctcctacctttggctgcggcaccaaggtggaaatcaaggagcccaagagcagcgacaagacccacaccagcccccccagccccgcccccgagctgaacggcatttggagcgagtggagcgaagccagaagctgggataccgagagcgtgctgcctatgtgggtgctcgccctgatcgtgatcttcctgacaatcgccgtgctgctggccctgagattctgtggcatctacgagaaagtcaagaccctgaaggcccagaacagcgagctggcctctaccgccaacatgctgagagaacaggtggcccaactgggctacagactgcggcggaagtgggaagagaagatccccaatcctagcaagagccacctgttccagaatggctctgccgagctttggcctcctggcagcatgagcgcctttacatctggcagccctccacatcaaggcccctggggaagcagattccctgaactggaaggcgtgttccctgtcggcttcggcgatagcgaagtgtcccctctgaccatcgaggaccctaagcacgtgtgcgatcctccaagcggccctgatacaacaccagccgcaagcgatctgcctaccgagcaacctcctagtcctcagcctggacctcctgccgcttctcacacacctgagaaacaggccagctccttcgacttcaacggcccctatctgggccctcctcatagcagaagcctgcctgacattctgggccagcctgagcctcctcaagaaggcggatctcagaagtctcctccacctggcagcctggaatacctgtgtttgcctgctggtggccaggtgcagctggttcctcttgctcaagctatgggacctggacaggccgtggaagtggaaagaaggccatctcaaggcgccgctggctctccatctcttgaatctggcggaggacctgctcctcctgctctgggacctagagttggaggccaggaccagaaagacagccccgtggccattcctatgagcagcggagataccgaagatcccggcgtggcctctggctatgtgtcatctgccgacctggtgttcacccctaatagcggcgctagctccgtgtctctggtgcctagtcttggcctgcctagcgatcagaccccttctctgtgtcctggactggcttctggaccacctggtgctccaggacctgtgaagtctggctttgagggctacgtggaactgcctcctatcgagggcagaagccctagaagccccagaaacaaccctgtgcctcctgaggccaagtctcccgttctgaaccctggcgaaagacccgccgatgtgtctcctacatctcctcagccagagggactgctggtgctgcaacaagtgggcgactactgcttcctgcctggacttggaccaggacctctgagcctgagaagcaagcctagctctcctggacctgggcctgagatcaagaatctggaccaggccttccaagtgaagaagcctccaggccaggctgtgcctcaggtgccagtgatccagctgttcaaggccctgaagcagcaggactacctgagcctgccaccttgggaagtgaacaagccaggcgaagtgtgcgccaccaatttcagcctgctgaaacaggctggcgacgtggaagagaaccctggacctatggagaccgacaccctgatcctgtgggtgctgctgctgctggtgcccggcagcaccggccagctccaggtgcaagagtctggacctggcctggtcaagcctagcgagacactgagcctgacctgtacagtgtctggcggcagcatcagcaacagctactttagctggggctggatcagacagcctcctggcaagtgcctggaatggatcggcagcttctactacggcgagaaaacctactacaaccccagcctgaagtcccgggccaccatcagcatcgataccagcaagagccagttctccctgaagctgagcagcgtgacagccgccgatacagccgtgtactactgtcctagaggccccaccatgatccggggcgtgatagattcttggggccagggcacactggtcaccgtttcttctgagccgaaatcctccgataaaacgcatacatccccgccatcaccagcgccagaactgaactggtcctcttggagcgaggccatcgagttcggcagcgacgatggcaatctgggcagcgtgtacatctacgtgctgctgatcgtgggcaccctcgtgtgtggaatcgtgctgggcttcctgttcaagcggttcctgagagccgagacagaccagctggaagatgagaagtctgccctccagaccgagatcgccaacctgctgaaagagaaagagaagctgatccagcggctgttccctccagtgcctcagatcaaggacaagctgaacgacaaccacgaggtcgaggacgagatcatctgggaagagttcacccctgaggaaggcaagggctacagagaagaggtgctgaccgtgaaagagatcacc

SEQ ID NO: 20 - DNA, 3270 bp, synthetic construct
  source 1..3270: mol_type = other DNA
atgggcaccagcctgctgtgctggatggccctgtgcctgctgggcgccgaccacgccgatgcctacccctacgacgtgcccgactacgccagcggagggggaggcagccccaccaccaccccagcccccagacctcccaccccagcccccaccatcgccagccagcctctgagcctgagacccgaggcctgccgcccagcggccggcggcgccgtgcacaccagaggcctggatttcgcctgcgatatctacatctgggccccactcgccggcacctgtggcgtgctgctgctgagcctggtgatcaccctgtactgcaaccaccgcaaccgcaggcgcgtgtgcaagtgccccaggcccgtcgtggaaggcagaggcagcctgcttacatgtggcgacgtggaagagaaccccggacctatgggctggagctgcatcatcctgttcctggtggccaccgccaccggcgtgcacagcgagatcgtgctgacacagtctcccgccacactgtcactgtctccaggcgaaagagccacactgagctgtagagcctctcagagcgtgcggtcttacctggcctggtatcagcagaagcctggacaggctccccggctgctgatctacgatgccagcaatagagccacaggcatccccgccagattttctggctctggcagcggcaccgatttcaccctgaccataagcagcctggaacctgaggacttcgccgtgtactactgccagcagagaagcaactggcctcctacctttggctgcggcaccaaggtggaaatcaaggagcccaagagcagcgacaagacccacaccagcccccccagccccgcccccgagctgaactggtcctcttggagcgaggccatcgagttcggcagcgacgatggcaatctgggcagcgtgtacatctacgtgctgctgatcgtgggcaccctcgtgtgtggaatcgtgctgggcttcctgttcaagcggttcctgagagccgagacagaccagctggaagatgagaagtctgccctccagaccgagatcgccaacctgctgaaagagaaagagaagctgatccagcggctgttccctccagtgcctcagatcaaggacaagctgaacgacaaccacgaggtcgaggacgagatcatctgggaagagttcacccctgaggaaggcaagggctacagagaagaggtgctgaccgtgaaagagatcaccgccaccaatttcagcctgctgaaacaggctggcgacgtggaagagaaccctggacctatggagaccgacaccctgatcctgtgggtgctgctgctgctggtgcccggcagcaccggccagctccaggtgcaagagtctggacctggcctggtcaagcctagcgagacactgagcctgacctgtacagtgtctggcggcagcatcagcaacagctactttagctggggctggatcagacagcctcctggcaagtgcctggaatggatcggcagcttctactacggcgagaaaacctactacaaccccagcctgaagtcccgggccaccatcagcatcgataccagcaagagccagttctccctgaagctgagcagcgtgacagccgccgatacagccgtgtactactgtcctagaggccccaccatgatccggggcgtgatagattcttggggccagggcacactggtcaccgtttcttctgagccgaaatcctccgataaaacgcatacatccccgccatcaccagcgccagaactgaacggcatttggagcgagtggagcgaagccagaagctgggataccgagagcgtgctgcctatgtgggtgctcgccctgatcgtgatcttcctgacaatcgccgtgctgctggccctgagattctgtggcatctacgagaaagtcaagaccctgaaggcccagaacagcgagctggcctctaccgccaacatgctgagagaacaggtggcccaactgggctacagactgcggcggaagtgggaagagaagatccccaatcctagcaagagccacctgttccagaatggctctgccgagctttggcctcctggcagcatgagcgcctttacatctggcagccctccacatcaaggcccctggggaagcagattccctgaactggaaggcgtgttccctgtcggcttcggcgatagcgaagtgtcccctctgaccatcgaggaccctaagcacgtgtgcgatcctccaagcggccctgatacaacaccagccgcaagcgatctgcctaccgagcaacctcctagtcctcagcctggacctcctgccgcttctcacacacctgagaaacaggccagctccttcgacttcaacggcccctatctgggccctcctcatagcagaagcctgcctgacattctgggccagcctgagcctcctcaagaaggcggatctcagaagtctcctccacctggcagcctggaatacctgtgtttgcctgctggtggccaggtgcagctggttcctcttgctcaagctatgggacctggacaggccgtggaagtggaaagaaggccatctcaaggcgccgctggctctccatctcttgaatctggcggaggacctgctcctcctgctctgggacctagagttggaggccaggaccagaaagacagccccgtggccattcctatgagcagcggagataccgaagatcccggcgtggcctctggctatgtgtcatctgccgacctggtgttcacccctaatagcggcgctagctccgtgtctctggtgcctagtcttggcctgcctagcgatcagaccccttctctgtgtcctggactggcttctggaccacctggtgctccaggacctgtgaagtctggctttgagggctacgtggaactgcctcctatcgagggcagaagccctagaagccccagaaacaaccctgtgcctcctgaggccaagtctcccgttctgaaccctggcgaaagacccgccgatgtgtctcctacatctcctcagccagagggactgctggtgctgcaacaagtgggcgactactgcttcctgcctggacttggaccaggacctctgagcctgagaagcaagcctagctctcctggacctgggcctgagatcaagaatctggaccaggccttccaagtgaagaagcctccaggccaggctgtgcctcaggtgccagtgatccagctgttcaaggccctgaagcagcaggactacctgagcctgccaccttgggaagtgaacaagccaggcgaagtgtgc

SEQ ID NO: 21 - DNA, 1626 bp, synthetic construct
  source 1..1626: mol_type = other DNA
atgggcaccagcctgctgtgctggatggccctgtgcctgctgggcgccgaccacgccgatgcctacccctacgacgtgcccgactacgccagcggagggggaggcagccccaccaccaccccagcccccagacctcccaccccagcccccaccatcgccagccagcctctgagcctgagacccgaggcctgccgcccagcggccggcggcgccgtgcacaccagaggcctggatttcgcctgcgatatctacatctgggccccactcgccggcacctgtggcgtgctgctgctgagcctggtgatcaccctgtactgcaaccaccgcaaccgcaggcgcgtgtgcaagtgccccaggcccgtcgtggaaggcagaggcagcctgcttacatgtggcgacgtggaagagaaccccggacctatgggctggagctgcatcatcctgttcctggtggccaccgccaccggcgtgcacagcgagatcgtgctgacacagtctcccgccacactgtcactgtctccaggcgaaagagccacactgagctgtagagcctctcagagcgtgcggtcttacctggcctggtatcagcagaagcctggacaggctccccggctgctgatctacgatgccagcaatagagccacaggcatccccgccagattttctggctctggcagcggcaccgatttcaccctgaccataagcagcctggaacctgaggacttcgccgtgtactactgccagcagagaagcaactggcctcctacctttggctgcggcaccaaggtggaaatcaaggagcccaagagcagcgacaagacccacaccagcccccccagccccgcccccgagctgagccctctgtttcccggacctagcaagcctttctgggtgctcgttgttgttggcggcgtgctggcctgttacagcctgctggttaccgtggccttcatcatcttttgggtccgaagcgccaccaatttcagcctgctgaaacaggctggcgacgtggaagagaaccctggacctatggagaccgacaccctgatcctgtgggtgctgctgctgctggtgcccggcagcaccggccagctccaggtgcaagagtctggacctggcctggtcaagcctagcgagacactgagcctgacctgtacagtgtctggcggcagcatcagcaacagctactttagctggggctggatcagacagcctcctggcaagtgcctggaatggatcggcagcttctactacggcgagaaaacctactacaaccccagcctgaagtcccgggccaccatcagcatcgataccagcaagagccagttctccctgaagctgagcagcgtgacagccgccgatacagccgtgtactactgtcctagaggccccaccatgatccggggcgtgatagattcttggggccagggcacactggtcaccgtttcttctgagccgaaatcctccgataaaacgcatacatccccgccatcaccagcgccagaactgtccccgctcttcccgggcccctccaaaccgttttgggtcctggtggtagtaggtggtgtcttggcgtgctattccctcctcgtgacggtcgcgtttattattttctgggtgcgctcc

SEQ ID NO: 22 - DNA, 1608 bp, synthetic construct
  source 1..1608: mol_type = other DNA
atgggcaccagcctgctgtgctggatggccctgtgcctgctgggcgccgaccacgccgatgcctacccctacgacgtgcccgactacgccagcggagggggaggcagccccaccaccaccccagcccccagacctcccaccccagcccccaccatcgccagccagcctctgagcctgagacccgaggcctgccgcccagcggccggcggcgccgtgcacaccagaggcctggatttcgcctgcgatatctacatctgggccccactcgccggcacctgtggcgtgctgctgctgagcctggtgatcaccctgtactgcaaccaccgcaaccgcaggcgcgtgtgcaagtgccccaggcccgtcgtggaaggcagaggcagcctgcttacatgtggcgacgtggaagagaaccccggacctatgggctggagctgcatcatcctgttcctggtggccaccgccaccggcgtgcacagcgagatcgtgctgacacagtctcccgccacactgtcactgtctccaggcgaaagagccacactgagctgtagagcctctcagagcgtgcggtcttacctggcctggtatcagcagaagcctggacaggctccccggctgctgatctacgatgccagcaatagagccacaggcatccccgccagattttctggctctggcagcggcaccgatttcaccctgaccataagcagcctggaacctgaggacttcgccgtgtactactgccagcagagaagcaactggcctcctacctttggctgcggcaccaaggtggaaatcaaggagcccaagagcagcgacaagacccacaccagcccccccagccccgcccccgagctgaacaacagctccggcgaaatggaccctatcctgctgacaatcagcatcctgagctttttcagcgtggccctgctggtcatcctggcctgtgtgctgtggaagaaggccaccaatttcagcctgctgaaacaggctggcgacgtggaagagaaccctggacctatggagaccgacaccctgatcctgtgggtgctgctgctgctggtgcccggcagcaccggccagctccaggtgcaagagtctggacctggcctggtcaagcctagcgagacactgagcctgacctgtacagtgtctggcggcagcatcagcaacagctactttagctggggctggatcagacagcctcctggcaagtgcctggaatggatcggcagcttctactacggcgagaaaacctactacaaccccagcctgaagtcccgggccaccatcagcatcgataccagcaagagccagttctccctgaagctgagcagcgtgacagccgccgatacagccgtgtactactgtcctagaggccccaccatgatccggggcgtgatagattcttggggccagggcacactggtcaccgtttcttctgagccgaaatcctccgataaaacgcatacatccccgccatcaccagcgccagaactgaacaccagcaaggaaaacccctttctgttcgccctggaggccgtggttatcagcgtgggctctatgggcctgatcatcagcctgctgtgcgtgtacttctggctggaacgg

SEQ ID NO: 23 - DNA, 3129 bp, synthetic construct
  source 1..3129: mol_type = other DNA
atgggcaccagcctgctgtgctggatggccctgtgcctgctgggcgccgaccacgccgatgcctacccctacgacgtgcccgactacgccagcggagggggaggcagccccaccaccaccccagcccccagacctcccaccccagcccccaccatcgccagccagcctctgagcctgagacccgaggcctgccgcccagcggccggcggcgccgtgcacaccagaggcctggatttcgcctgcgatatctacatctgggccccactcgccggcacctgtggcgtgctgctgctgagcctggtgatcaccctgtactgcaaccaccgcaaccgcaggcgcgtgtgcaagtgccccaggcccgtcgtggaaggcagaggcagcctgcttacatgtggcgacgtggaagagaaccccggacctatgggctggagctgcatcatcctgttcctggtggccaccgccaccggcgtgcacagcagaacagtggccgctcctagcgtgttcatcttcccaccttccgacgagcagctgaagtctggcacagcctctgtcgtgtgcctgctgaacaacttctaccccagagaagccaaggtgcagtggaaggtggacaacgccctccagagcggcaatagccaagagagcgtgaccgagcaggacagcaaggactctacctacagcctgagcagcaccctgacactgagcaaggccgactacgagaagcacaaagtgtacgcctgcgaagtgacccaccagggcctttctagccctgtgaccaagagcttcaaccggggagagtgcgagcccaagagctgcgataagacccacacctgtcctccatgtcctgagcccaagagcagcgacaagacccacaccagcccccccagccccgcccccgagctgaacggcatttggagcgagtggagcgaagccagaagctgggataccgagagcgtgctgcctatgtgggtgctcgccctgatcgtgatcttcctgacaatcgccgtgctgctggccctgagattctgtggcatctacggctacagactgcggcggaagtgggaagagaagatccccaatcctagcaagagccacctgttccagaatggctctgccgagctttggcctcctggcagcatgagcgcctttacatctggcagccctccacatcaaggcccctggggaagcagattccctgaactggaaggcgtgttccctgtcggcttcggcgatagcgaagtgtcccctctgaccatcgaggaccctaagcacgtgtgcgatcctccaagcggccctgatacaacaccagccgcaagcgatctgcctaccgagcaacctcctagtcctcagcctggacctcctgccgcttctcacacacctgagaaacaggccagctccttcgacttcaacggcccctatctgggccctcctcatagcagaagcctgcctgacattctgggccagcctgagcctcctcaagaaggcggatctcagaagtctcctccacctggcagcctggaatacctgtgtttgcctgctggtggccaggtgcagctggttcctcttgctcaagctatgggacctggacaggccgtggaagtggaaagaaggccatctcaaggcgccgctggctctccatctcttgaatctggcggaggacctgctcctcctgctctgggacctagagttggaggccaggaccagaaagacagccccgtggccattcctatgagcagcggagataccgaagatcccggcgtggcctctggctatgtgtcatctgccgacctggtgttcacccctaatagcggcgctagctccgtgtctctggtgcctagtcttggcctgcctagcgatcagaccccttctctgtgtcctggactggcttctggaccacctggtgctccaggacctgtgaagtctggctttgagggctacgtggaactgcctcctatcgagggcagaagccctagaagccccagaaacaaccctgtgcctcctgaggccaagtctcccgttctgaaccctggcgaaagacccgccgatgtgtctcctacatctcctcagccagagggactgctggtgctgcaacaagtgggcgactactgcttcctgcctggacttggaccaggacctctgagcctgagaagcaagcctagctctcctggacctgggcctgagatcaagaatctggaccaggccttccaagtgaagaagcctccaggccaggctgtgcctcaggtgccagtgatccagctgttcaaggccctgaagcagcaggactacctgagcctgccaccttgggaagtgaacaagccaggcgaagtgtgcgccaccaatttcagcctgctgaaacaggctggcgacgtggaagagaaccctggacctatggagaccgacaccctgatcctgtgggtgctgctgctgctggtgcccggcagcaccggcgcctctacaaagggccctagtgtgttccctctggctcccagcagcaagtctacatctggcggaacagccgctctgggctgcctggtcaaggattactttcccgagcctgtgaccgtgtcctggaatagcggagcactgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgacaagacccacacctgtcctccatgtcctgagccgaaatcctccgataaaacgcatacatccccgccatcaccagcgccagaactgaactggtcctcttggagcgaggccatcgagttcggcagcgacgatggcaatctgggcagcgtgtacatctacgtgctgctgatcgtgggcaccctcgtgtgtggaatcgtgctgggcttcctgttcaagcggttcctgagaatccagcggctgttccctccagtgcctcagatcaaggacaagctgaacgacaaccacgaggtcgaggacgagatcatctgggaagagttcacccctgaggaaggcaagggctacagagaagaggtgctgaccgtgaaagagatcacc

SEQ ID NO: 24 - DNA, 3096 bp, synthetic construct
  source 1..3096: mol_type = other DNA
atgggcaccagcctgctgtgctggatggccctgtgcctgctgggcgccgaccacgccgatgcctacccctacgacgtgcccgactacgccagcggagggggaggcagccccaccaccaccccagcccccagacctcccaccccagcccccaccatcgccagccagcctctgagcctgagacccgaggcctgccgcccagcggccggcggcgccgtgcacaccagaggcctggatttcgcctgcgatatctacatctgggccccactcgccggcacctgtggcgtgctgctgctgagcctggtgatcaccctgtactgcaaccaccgcaaccgcaggcgcgtgtgcaagtgccccaggcccgtcgtggaaggcagaggcagcctgcttacatgtggcgacgtggaagagaaccccggacctatgggctggagctgcatcatcctgttcctggtggccaccgccaccggcgtgcacagcgacatccagatgacacagagccctagcagcctgtctgccagcgtgggagacagagtgaccatcacatgcaaggccagccagaacgtgggcatcaacgtggcctggtatcagcagaagcctggcaaggcccctaagagcctgatcagcagcgccagctacagatacagcggcgtgccaagcagattttccggctctggcagcggcaccgatttcaccctgacaatatccagcctccagcctgaggacttcgccacctacttttgccagcagtacgacacataccccttcaccttcggctgcggcaccaaggtggaaatcaaggagcccaagagcagcgacaagacccacaccagcccccccagccccgcccccgagctgaacggcatttggagcgagtggagcgaagccagaagctgggataccgagagcgtgctgcctatgtgggtgctcgccctgatcgtgatcttcctgacaatcgccgtgctgctggccctgagattctgtggcatctacggctacagactgcggcggaagtgggaagagaagatccccaatcctagcaagagccacctgttccagaatggctctgccgagctttggcctcctggcagcatgagcgcctttacatctggcagccctccacatcaaggcccctggggaagcagattccctgaactggaaggcgtgttccctgtcggcttcggcgatagcgaagtgtcccctctgaccatcgaggaccctaagcacgtgtgcgatcctccaagcggccctgatacaacaccagccgcaagcgatctgcctaccgagcaacctcctagtcctcagcctggacctcctgccgcttctcacacacctgagaaacaggccagctccttcgacttcaacggcccctatctgggccctcctcatagcagaagcctgcctgacattctgggccagcctgagcctcctcaagaaggcggatctcagaagtctcctccacctggcagcctggaatacctgtgtttgcctgctggtggccaggtgcagctggttcctcttgctcaagctatgggacctggacaggccgtggaagtggaaagaaggccatctcaaggcgccgctggctctccatctcttgaatctggcggaggacctgctcctcctgctctgggacctagagttggaggccaggaccagaaagacagccccgtggccattcctatgagcagcggagataccgaagatcccggcgtggcctctggctatgtgtcatctgccgacctggtgttcacccctaatagcggcgctagctccgtgtctctggtgcctagtcttggcctgcctagcgatcagaccccttctctgtgtcctggactggcttctggaccacctggtgctccaggacctgtgaagtctggctttgagggctacgtggaactgcctcctatcgagggcagaagccctagaagccccagaaacaaccctgtgcctcctgaggccaagtctcccgttctgaaccctggcgaaagacccgccgatgtgtctcctacatctcctcagccagagggactgctggtgctgcaacaagtgggcgactactgcttcctgcctggacttggaccaggacctctgagcctgagaagcaagcctagctctcctggacctgggcctgagatcaagaatctggaccaggccttccaagtgaagaagcctccaggccaggctgtgcctcaggtgccagtgatccagctgttcaaggccctgaagcagcaggactacctgagcctgccaccttgggaagtgaacaagccaggcgaagtgtgcgccaccaatttcagcctgctgaaacaggctggcgacgtggaagagaaccctggacctatggagaccgacaccctgatcctgtgggtgctgctgctgctggtgcccggcagcaccggccaggttcagctggttcagtctggcgccgaggaagtgaagaaacctggcagctctgtgaaggtgtcctgcaaggccagcggctacacctttaccacctactacctgcactgggtccgacaggctccaggccaatgtctggaatggatgggctggatctaccccggcaatgtgcacgcccagtacaacgagaagttcaagggcagagtgaccatcaccgccgacaagagcacaagcaccgcctacatggaactgagcagcctgagaagcgaggacaccgccgtgtactactgtgccagaagctgggagggcttcccttattggggccagggaaccaccgtgaccgttagctctgagccgaaatcctccgataaaacgcatacatccccgccatcaccagcgccagaactgaactggtcctcttggagcgaggccatcgagttcggcagcgacgatggcaatctgggcagcgtgtacatctacgtgctgctgatcgtgggcaccctcgtgtgtggaatcgtgctgggcttcctgttcaagcggttcctgagaatccagcggctgttccctccagtgcctcagatcaaggacaagctgaacgacaaccacgaggtcgaggacgagatcatctgggaagagttcacccctgaggaaggcaagggctacagagaagaggtgctgaccgtgaaagagatcacc

SEQ ID NO: 25 - protein, 107 aa, synthetic construct
EIVLTQSPATLSLSPGERATLSCRASQSVRSYLAWYQQKPGQAPRLLIYDASNRATGIPARFSGSGSGTDFTLTISSLEPEDFAVYYCQQRSNWPPTFGCGTKVEIK

SEQ ID NO: 26 - protein, 19 aa, synthetic construct
EPKSSDKTHTSPPSPAPEL

SEQ ID NO: 27 - protein, 14 aa, synthetic construct
NTSKENPFLFALEA

SEQ ID NO: 28 - protein, 21 aa, synthetic construct
VVISVGSMGLIISLLCVYFWL

SEQ ID NO: 29 - protein, 107 aa, synthetic construct
VVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 30 - protein, 19 aa, synthetic construct
ATNFSLLKQAGDVEENPGP

SEQ ID NO: 31 - protein, 121 aa, synthetic construct
QLQVQESGPGLVKPSETLSLTCTVSGGSISNSYFSWGWIRQPPGKCLEWIGSFYYGEKTYYNPSLKSRATISIDTSKSQFSLKLSSVTAADTAVYYCPRGPTMIRGVIDSWGQGTLVTVSS

SEQ ID NO: 32 - protein, 19 aa, synthetic construct
EPKSSDKTHTSPPSPAPEL

SEQ ID NO: 33 - protein, 8 aa, synthetic construct
NNSSGEMD

SEQ ID NO: 34 - protein, 25 aa, synthetic construct
PILLTISILSFFSVALLVILACVLW

SEQ ID NO: 35 - protein, 195 aa, synthetic construct
KKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 36 - protein, 27 aa, synthetic construct
EKVKTLKAQNSELASTANMLREQVAQL

SEQ ID NO: 37 - protein, 27 aa, synthetic construct
AETDQLEDEKSALQTEIANLLKEKEKL

SEQ ID NO: 38 - protein, 22 aa, synthetic construct
NGIWSEWSEARSWDTESVLPMW

SEQ ID NO: 39 - protein, 17 aa, synthetic construct
VLALIVIFLTIAVLLAL

SEQ ID NO: 40 - protein, 437 aa, synthetic construct
RFCGIYGYRLRRKWEEKIPNPSKSHLFQNGSAELWPPGSMSAFTSGSPPHQGPWGSRFPELEGVFPVGFGDSEVSPLTIEDPKHVCDPPSGPDTTPAASDLPTEQPPSPQPGPPAASHTPEKQASSFDFNGPYLGPPHSRSLPDILGQPEPPQEGGSQKSPPPGSLEYLCLPAGGQVQLVPLAQAMGPGQAVEVERRPSQGAAGSPSLESGGGPAPPALGPRVGGQDQKDSPVAIPMSSGDTEDPGVASGYVSSADLVFTPNSGASSVSLVPSLGLPSDQTPSLCPGLASGPPGAPGPVKSGFEGYVELPPIEGRSPRSPRNNPVPPEAKSPVLNPGERPADVSPTSPQPEGLLVLQQVGDYCFLPGLGPGPLSLRSKPSSPGPGPEIKNLDQAFQVKKPPGQAVPQVPVIQLFKALKQQDYLSLPPWEVNKPGEVC

SEQ ID NO: 41 - protein, 16 aa, synthetic construct
NWSSWSEAIEFGSDDG

SEQ ID NO: 42 - protein, 26 aa, synthetic construct
NLGSVYIYVLLIVGTLVCGIVLGFLF

SEQ ID NO: 43 - protein, 54 aa, synthetic construct
KRFLRIQRLFPPVPQIKDKLNDNHEVEDEIIWEEFTPEEGKGYREEVLTVKEIT

SEQ ID NO: 44 - protein, 9 aa, synthetic construct
SPLFPGPSK

SEQ ID NO: 45 - protein, 28 aa, synthetic construct
PFWVLVVVGGVLACYSLLVTVAFIIFWV

SEQ ID NO: 46 - protein, 10 aa, synthetic construct
SPLFPGPSKP

SEQ ID NO: 47 - protein, 27 aa, synthetic construct
FWVLVVVGGVLACYSLLVTVAFIIFWV

SEQ ID NO: 48 - protein, 107 aa, synthetic construct
DIQMTQSPSSLSASVGDRVTITCKASQNVGINVAWYQQKPGKAPKSLISSASYRYSGVPSRFSGSGSGTDFTLTISSLQPEDFATYFCQQYDTYPFTFGCGTKVEIK

SEQ ID NO: 49 - protein, 117 aa, synthetic construct
QVQLVQSGAEEVKKPGSSVKVSCKASGYTFTTYYLHWVRQAPGQCLEWMGWIYPGNVHAQYNEKFKGRVTITADKSTSTAYMELSSLRSEDTAVYYCARSWEGFPYWGQGTTVTVSS

SEQ ID NO: 50 - protein, 122 aa, synthetic construct
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEPKSCDKTHTCPPCP

SEQ ID NO: 51 - protein, 113 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCP

SEQ ID NO: 52 - DNA, 321 bp, synthetic construct
  source 1..321: mol_type = other DNA
gagatcgtgctgacacagtctcccgccacactgtcactgtctccaggcgaaagagccacactgagctgtagagcctctcagagcgtgcggtcttacctggcctggtatcagcagaagcctggacaggctccccggctgctgatctacgatgccagcaatagagccacaggcatccccgccagattttctggctctggcagcggcaccgatttcaccctgaccataagcagcctggaacctgaggacttcgccgtgtactactgccagcagagaagcaactggcctcctacctttggctgcggcaccaaggtggaaatcaag

SEQ ID NO: 53 - DNA, 57 bp, synthetic construct
  source 1..57: mol_type = other DNA
gagcccaagagcagcgacaagacccacaccagcccccccagccccgcccccgagctg

SEQ ID NO: 54 - DNA, 42 bp, synthetic construct
  source 1..42: mol_type = other DNA
aacaccagcaaggaaaacccctttctgttcgccctggaggcc

SEQ ID NO: 55 - DNA, 63 bp, synthetic construct
  source 1..63: mol_type = other DNA
gtggttatcagcgtgggctctatgggcctgatcatcagcctgctgtgcgtgtacttctggctg

SEQ ID NO: 56 - DNA, 258 bp, synthetic construct
  source 1..258: mol_type = other DNA
gaacggaccatgcctagaatccccacactgaagaaccttgaggacctggtcaccgagtaccacggcaacttttctgcttggagcggcgtgtccaaaggcctggccgaatctctccagcctgactacagcgagagactgtgcctggtgtctgagatccctcctaaaggcggagccctcggagaaggacctggcgcctctccatgtaatcagcacagcccttactgggctcctccttgctacaccctgaagcctgagaca

SEQ ID NO: 57 - DNA, 57 bp, synthetic construct
  source 1..57: mol_type = other DNA
gccaccaatttcagcctgctgaaacaggctggcgacgtggaagagaaccctggacct

SEQ ID NO: 58 - DNA, 363 bp, synthetic construct
  source 1..363: mol_type = other DNA
cagctccaggtgcaagagtctggacctggcctggtcaagcctagcgagacactgagcctgacctgtacagtgtctggcggcagcatcagcaacagctactttagctggggctggatcagacagcctcctggcaagtgcctggaatggatcggcagcttctactacggcgagaaaacctactacaaccccagcctgaagtcccgggccaccatcagcatcgataccagcaagagccagttctccctgaagctgagcagcgtgacagccgccgatacagccgtgtactactgtcctagaggccccaccatgatccggggcgtgatagattcttggggccagggcacactggtcaccgtttcttct

SEQ ID NO: 59 - DNA, 57 bp, synthetic construct
  source 1..57: mol_type = other DNA
gagccgaaatcctccgataaaacgcatacatccccgccatcaccagcgccagaactg

SEQ ID NO: 60 - DNA, 24 bp, synthetic construct
  source 1..24: mol_type = other DNA
aacaacagctccggcgaaatggac

SEQ ID NO: 61 - DNA, 75 bp, synthetic construct
  source 1..75: mol_type = other DNA
cctatcctgctgacaatcagcatcctgagctttttcagcgtggccctgctggtcatcctggcctgtgtgctgtgg

SEQ ID NO: 62 - DNA, 585 bp, synthetic construct
  source 1..585: mol_type = other DNA
aagaagcggatcaagcccatcgtgtggcccagcctgcctgaccacaagaaaaccctggaacacctgtgcaagaagccccggaagaacctgaacgtgtccttcaatcccgagagcttcctggactgccagatccacagagtggacgacatccaggccagggacgaagtggaaggcttcctccaggacacattccctcagcagctggaagagagcgagaagcagagactcggcggagatgtgcagagccctaattgccctagcgaggacgtggtcatcacccctgagagcttcggcagagatagcagcctgacatgtctggccggcaatgtgtccgcctgtgatgcccctatcctgagcagtagcagaagcctggattgcagagagagcggcaagaacggccctcacgtgtaccaggatctgctgctgtctctgggcaccaccaatagcacactgcctccacctttcagcctgcaaagcggcatcctgacactgaaccctgtggctcagggacagccaatcctgacaagcctgggcagcaatcaagaagaggcctacgtcaccatgagcagcttctaccagaaccag

SEQ ID NO: 63 - DNA, 81 bp, synthetic construct
  source 1..81: mol_type = other DNA
gagaaagtcaagaccctgaaggcccagaacagcgagctggcctctaccgccaacatgctgagagaacaggtggcccaactg

SEQ ID NO: 64 - DNA, 81 bp, synthetic construct
  source 1..81: mol_type = other DNA
gccgagacagaccagctggaagatgagaagtctgccctccagaccgagatcgccaacctgctgaaagagaaagagaagctg

SEQ ID NO: 65 - DNA, 66 bp, synthetic construct
  source 1..66: mol_type = other DNA
aacggcatttggagcgagtggagcgaagccagaagctgggataccgagagcgtgctgcctatgtgg

SEQ ID NO: 66 - DNA, 51 bp, synthetic construct
  source 1..51: mol_type = other DNA
gtgctcgccctgatcgtgatcttcctgacaatcgccgtgctgctggccctg

SEQ ID NO: 67 - DNA, 1311 bp, synthetic construct
  source 1..1311: mol_type = other DNA
agattctgtggcatctacggctacagactgcggcggaagtgggaagagaagatccccaatcctagcaagagccacctgttccagaatggctctgccgagctttggcctcctggcagcatgagcgcctttacatctggcagccctccacatcaaggcccctggggaagcagattccctgaactggaaggcgtgttccctgtcggcttcggcgatagcgaagtgtcccctctgaccatcgaggaccctaagcacgtgtgcgatcctccaagcggccctgatacaacaccagccgcaagcgatctgcctaccgagcaacctcctagtcctcagcctggacctcctgccgcttctcacacacctgagaaacaggccagctccttcgacttcaacggcccctatctgggccctcctcatagcagaagcctgcctgacattctgggccagcctgagcctcctcaagaaggcggatctcagaagtctcctccacctggcagcctggaatacctgtgtttgcctgctggtggccaggtgcagctggttcctcttgctcaagctatgggacctggacaggccgtggaagtggaaagaaggccatctcaaggcgccgctggctctccatctcttgaatctggcggaggacctgctcctcctgctctgggacctagagttggaggccaggaccagaaagacagccccgtggccattcctatgagcagcggagataccgaagatcccggcgtggcctctggctatgtgtcatctgccgacctggtgttcacccctaatagcggcgctagctccgtgtctctggtgcctagtcttggcctgcctagcgatcagaccccttctctgtgtcctggactggcttctggaccacctggtgctccaggacctgtgaagtctggctttgagggctacgtggaactgcctcctatcgagggcagaagccctagaagccccagaaacaaccctgtgcctcctgaggccaagtctcccgttctgaaccctggcgaaagacccgccgatgtgtctcctacatctcctcagccagagggactgctggtgctgcaacaagtgggcgactactgcttcctgcctggacttggaccaggacctctgagcctgagaagcaagcctagctctcctggacctgggcctgagatcaagaatctggaccaggccttccaagtgaagaagcctccaggccaggctgtgcctcaggtgccagtgatccagctgttcaaggccctgaagcagcaggactacctgagcctgccaccttgggaagtgaacaagccaggcgaagtgtgc

SEQ ID NO: 68 - DNA, 48 bp, synthetic construct
  source 1..48: mol_type = other DNA
aactggtcctcttggagcgaggccatcgagttcggcagcgacgatggc

SEQ ID NO: 69 - DNA, 78 bp, synthetic construct
  source 1..78: mol_type = other DNA
aatctgggcagcgtgtacatctacgtgctgctgatcgtgggcaccctcgtgtgtggaatcgtgctgggcttcctgttc

SEQ ID NO: 70 - DNA, 162 bp, synthetic construct
  source 1..162: mol_type = other DNA
aagcggttcctgagaatccagcggctgttccctccagtgcctcagatcaaggacaagctgaacgacaaccacgaggtcgaggacgagatcatctgggaagagttcacccctgaggaaggcaagggctacagagaagaggtgctgaccgtgaaagagatcacc

SEQ ID NO: 71 - DNA, 27 bp, synthetic construct
  source 1..27: mol_type = other DNA
agccctctgtttcccggacctagcaag

SEQ ID NO: 72 - DNA, 84 bp, synthetic construct
  source 1..84: mol_type = other DNA
cctttctgggtgctcgttgttgttggcggcgtgctggcctgttacagcctgctggttaccgtggccttcatcatcttttgggtc

SEQ ID NO: 73 - DNA, 30 bp, synthetic construct
  source 1..30: mol_type = other DNA
tccccgctcttcccgggcccctccaaaccg

SEQ ID NO: 74 - DNA, 81 bp, synthetic construct
  source 1..81: mol_type = other DNA
ttttgggtcctggtggtagtaggtggtgtcttggcgtgctattccctcctcgtgacggtcgcgtttattattttctgggtg

SEQ ID NO: 75 - DNA, 321 bp, synthetic construct
  source 1..321: mol_type = other DNA
gacatccagatgacacagagccctagcagcctgtctgccagcgtgggagacagagtgaccatcacatgcaaggccagccagaacgtgggcatcaacgtggcctggtatcagcagaagcctggcaaggcccctaagagcctgatcagcagcgccagctacagatacagcggcgtgccaagcagattttccggctctggcagcggcaccgatttcaccctgacaatatccagcctccagcctgaggacttcgccacctacttttgccagcagtacgacacataccccttcaccttcggctgcggcaccaaggtggaaatcaag

SEQ ID NO: 76 - DNA, 351 bp, synthetic construct
  source 1..351: mol_type = other DNA
caggttcagctggttcagtctggcgccgaggaagtgaagaaacctggcagctctgtgaaggtgtcctgcaaggccagcggctacacctttaccacctactacctgcactgggtccgacaggctccaggccaatgtctggaatggatgggctggatctaccccggcaatgtgcacgcccagtacaacgagaagttcaagggcagagtgaccatcaccgccgacaagagcacaagcaccgcctacatggaactgagcagcctgagaagcgaggacaccgccgtgtactactgtgccagaagctgggagggcttcccttattggggccagggaaccaccgtgaccgttagctct

SEQ ID NO: 77 - DNA, 366 bp, synthetic construct
  source 1..366: mol_type = other DNA
agaacagtggccgctcctagcgtgttcatcttcccaccttccgacgagcagctgaagtctggcacagcctctgtcgtgtgcctgctgaacaacttctaccccagagaagccaaggtgcagtggaaggtggacaacgccctccagagcggcaatagccaagagagcgtgaccgagcaggacagcaaggactctacctacagcctgagcagcaccctgacactgagcaaggccgactacgagaagcacaaagtgtacgcctgcgaagtgacccaccagggcctttctagccctgtgaccaagagcttcaaccggggagagtgcgagcccaagagctgcgataagacccacacctgtcctccatgtcct

SEQ ID NO: 78 - DNA, 339 bp, synthetic construct
  source 1..339: mol_type = other DNA
gcctctacaaagggccctagtgtgttccctctggctcccagcagcaagtctacatctggcggaacagccgctctgggctgcctggtcaaggattactttcccgagcctgtgaccgtgtcctggaatagcggagcactgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgacaagacccacacctgtcctccatgtcct

SEQ ID NO: 79 - protein, 107 aa, synthetic construct
EIVLTQSPATLSLSPGERATLSCRASQSVRSYLAWYQQKPGQAPRLLIYDASNRATGIPARFSGSGSGTDFTLTISSLEPEDFAVYYCQQRSNWPPTFGQGTKVEIK

SEQ ID NO: 80 - protein, 121 aa, synthetic construct
QLQVQESGPGLVKPSETLSLTCTVSGGSISNSYFSWGWIRQPPGKGLEWIGSFYYGEKTYYNPSLKSRATISIDTSKSQFSLKLSSVTAADTAVYYCPRGPTMIRGVIDSWGQGTLVTVSS

SEQ ID NO: 81 - protein, 214 aa, synthetic construct
EIVLTQSPATLSLSPGERATLSCRASQSVRSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - protein, 224 aa, synthetic construct
QLQVQESGPGLVKPSETLSLTCTVSGGSISNSYFSWGWIRQPPGKGLEWIGSFYYGEKTYYNPSLKSRATISIDTSKSQFSLKLSSVTAADTAVYYCPRGPTMIRGVIDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 83 - protein, 121 aa, synthetic construct
QLQVQESGPGLVKPSETLSLTCTVSGGSISNSYFSWGWCRQPPGKGLEWIGSFYYGEKTYYNPSLKSRATISIDTSKSQFSLKLSSVTAADTAVYYCPRGPTMIRGVIDSWGQGTLVTVSS

SEQ ID NO: 84 - protein, 107 aa, synthetic construct
EIVLTQSPATLSLSPGERATLSCRASQSVRSYLAWYQQKPGQAPRLLIYDASNRATGIPARFSGSGSGTDFTLTISSLEPEDFAVYYCQQRSNWCPTFGGGTKVEIK

SEQ ID NO: 85 - protein, 121 aa, Homo sapiens; identical to SEQ ID NO: 31

SEQ ID NO: 86 - protein, 107 aa, synthetic construct
EIVLTQSPATLSLSPGERATLSCRASQSVRSYLAWYQQKPGQAPRLLIYDASNRATGIPARFSGSGSGTDFTLTISSLEPEDFAVYYCQQRSNWPPTFGGGTKVCIK

SEQ ID NO: 87 - protein, 121 aa, synthetic construct
QLQVQESGPGLVKPSETLSLTCTVSGGSISNSYFSWGWIRQPPGKGCEWIGSFYYGEKTYYNPSLKSRATISIDTSKSQFSLKLSSVTAADTAVYYCPRGPTMIRGVIDSWGQGTLVTVSS

SEQ ID NO: 88 - protein, 107 aa, synthetic construct
EIVLTQSPATLSLSPGERATLSCRASQSVRSYLAWYQQKPGQAPRLLIYDASNRATGIPARFSGSGSGTDFTLTISSLEPEDFAVYCCQQRSNWPPTFGGGTKVEIK

SEQ ID NO: 89 - protein, 121 aa, synthetic construct
QLQVQESGPGLVKPSETLSLTCTVSGGSISNSYFSWGWIRQPPGKGLEWIGSFYYGCKTYYNPSLKSRATISIDTSKSQFSLKLSSVTAADTAVYYCPRGPTMIRGVIDSWGQGTLVTVSS

SEQ ID NO: 90 - protein, 107 aa, synthetic construct
EIVLTQSPATLSLSPGERATLSCRASQSVRSYLAWYQQKPGQAPRLLIYDASNRATGIPARFSGSGSGTDFTLTISSLEPEDFAVYYCQQRSNWPPTFGGCTKVEIK

SEQ ID NO: 91 - protein, 121 aa, synthetic construct
QLQVQESGPGLVKPSETLSLTCTVSGGSISNSYFSWGWIRQPPGKGLEWIGSFYYGEKTYYNPSLKSRATISIDTSKSQFSLKLSSVTAADTAVYYCPRGPTMICGVIDSWGQGTLVTVSS

SEQ ID NO: 92 - protein, 107 aa, synthetic construct
EIVLTQSPATLSLSPGERATLSCRASQSVRSYLAWYQQKPGQAPRLLIYCASNRATGIPARFSGSGSGTDFTLTISSLEPEDFAVYYCQQRSNWPPTFGGGTKVEIK

SEQ ID NO: 93 - protein, 121 aa, synthetic construct
QLQVQESGPGLVKPSETLSLTCTVSGGSISNSYFSWGWIRQPPGKGLEWIGSFYYGEKTYYNPSLKSRATISIDTSKSQFSLKLSSVTAADTAVYYCPRGPTCIRGVIDSWGQGTLVTVSS

SEQ ID NO: 94 - protein, 107 aa, synthetic construct
EIVLTQSPATLSLSPGERATLSCRASQSVRSYLAWYQQKPGQAPRCLIYDASNRATGIPARFSGSGSGTDFTLTISSLEPEDFAVYYCQQRSNWPPTFGGGTKVEIK

SEQ ID NO: 95 - protein, 121 aa, synthetic construct
QLQVQESGPGLVKPSETLSLTCTVSGGSISNSYFSWGWIRQPPGKGLEWIGSFYYGEKTYYNPSLKSRATISIDTSKSQFSLKLSSVTAADTAVYYCPRGPTMIRGVIDSWGCGTLVTVSS

SEQ ID NO: 96 - protein, 107 aa, synthetic construct
EIVLTQSPATLSLSPGERATLSCRASQSVRSYLAWYQQKPGQCPRLLIYDASNRATGIPARFSGSGSGTDFTLTISSLEPEDFAVYYCQQRSNWPPTFGGGTKVEIK

(19) United States
(12) Patent Application Publication
(10) Pub. No.: US 2026/0325636 A1
(43) Pub. Date: Oct. 22, 2026

(54) DUAL BLOCKING AND CHIMERIC CYTOKINE RECEPTOR

(71) Applicant: Chimeris UK Ltd., Sawston, Cambridge (GB)

(72) Inventors: Rajesh Karattil, Sawston, Cambridge (GB); Saket Srivastava, Sawston, Cambridge (GB); Biao Ma, Sawston, Cambridge (GB); Shimobi Onuoha, Sawston, Cambridge (GB); Preeta Datta, Sawston, Cambridge (GB)

(21) Appl. No.: 19/483,186

(22) PCT Filed: May 9, 2024
(86) PCT No.: PCT/US2024/028658

§ 371 Date: Nov. 10, 2025

Related U.S. Application Data
(60) Provisional application No. 63/501,966, filed on May 12, 2023.

(51) Int. Cl. A61K 40/31; A61K 40/11; A61K 40/41; C07K 14/535; C07K 14/54; C07K 14/705; C07K 16/28; C12N 5/0783

(52) U.S. Cl. CPC A61K 40/31; A61K 40/11; A61K 40/41; C07K 14/535; C07K 14/5418; C07K 14/70521; C07K 16/2863; C12N 5/0636; C07K 2317/624; C07K 2319/02; C07K 2319/03; C12N 2510/00

(57) ABSTRACT

The present disclosure relates to a protein construct with a dimerization domain, the dimerization domain being strategically engineered for enhanced stabilization. The present disclosure also relates to methods of generating cytokine signaling comprising administering to a patient or system the protein construct, compositions for use in such methods, and further methods of use of the protein as part of T cell-based immunotherapy.

### RELATED APPLICATIONS AND INCORPORATION BY REFERENCE

This application claims the benefit of U.S. Provisional Ser. No. 63/501,966, filed May 12, 2023, which is hereby incorporated by reference in its entirety.

### REFERENCE TO SEQUENCE LISTING

The present application is being filed along with a Sequence Listing in electronic format. The Sequence Listing is provided as a file entitled CHMRS.002WO.xml, which was created and last modified on May 9, 2024, and is 136,066 bytes in size. The information in the electronic Sequence Listing is hereby incorporated by reference in its entirety.

### FIELD

Aspects of the present disclosure described herein relate to methods, cells, and compositions for enhancing the effectiveness of chimeric antigen receptor (CAR) bearing T-cells against solid tumors. In particular, embodiments provided herein relate to methods, cells, and compositions for CAR T-cell expressing a dimerized protein, wherein the protein is capable of performing the dual function of blocking an immunoinhibitory pathway while providing an independent constitutive cytokine signal.

### BACKGROUND

A variety of cellular therapies have become standardized in the treatment of cancer, including immunotherapy based on adoptive transfer of lymphocytes (e.g., T cells) into a patient. Among the many different types of immunotherapeutic agents, one of the most promising of the immunotherapeutic agents being developed is T cells expressing chimeric antigen receptors (CAR T cells). The chimeric antigen receptor (CAR) is a genetically engineered receptor that is designed to target a specific antigen, for example, a tumor antigen. The specific targeting can result in cytotoxicity against the tumor, for example, such that CAR T cells expressing CARs can target and kill tumors via the specific tumor antigens. CARs can include the infusion of polyclonal or antigen specific T-cells, lymphokine activated killer cells, natural killer cells, dendritic cells, or macrophages. Advancements have been made in the development of chimeric antigen receptor (CAR) bearing T-cells for adoptive T-cell therapies for cancer therapy, which are a promising therapeutic route for cancer immunotherapy and viral therapy.

CAR T-cell therapy is an immunotherapy in which the patient's own T-cells are isolated in a laboratory, genetically manipulated to express a synthetic receptor to recognize a particular antigen or protein, and reinfused into the patient. A CAR can include several domains. For example, without being limiting, the CAR can have: (1) an antigen-binding region, typically derived from an antibody; (2) a transmembrane domain to anchor the CAR into the T-cells; and/or (3) one or more intracellular T-cell signaling domains. First-generation CARs commonly incorporated a single chain variable fragment (scFv) that is derived from a monoclonal antibody (mAb), plus a signaling motif from a TCR ζ chain. The second- and third-generation CARs are an improvement over the first-generation CARs with co-stimulatory activating motifs, which can lead to the enhanced proliferation, cytotoxicity and persistence of the CAR bearing cells in vivo. Clinical trials have shown some evidence of anti-tumor activity, with insufficient activation, persistence, and homing to cancer tissue. Some anti-tumor responses have been reported in patients with B cell lymphoma, for example, and some neuroblastoma patients have reported partial response, stable disease, and remission. Second- and third-generation CAR-modified T-cells have been shown to be able to provide enhanced activation signals, proliferation, production of cytokines, and effector function of CAR-modified T-cells in pre-clinical trials. Initial clinical trials have been shown to exhibit some promising results.

### SUMMARY

Described herein are compositions and methods for treating diseases, the compositions including protein constructs that include a dimerization domain engineered for enhanced stabilization.

Accordingly, some embodiments provided herein relate to protein constructs. In some embodiments, the protein constructs are capable of dimerizing. In some embodiments, the protein constructs include a receptor-specific targeting region; a transmembrane domain; an intracellular signaling domain; and an Fv domain including at least one dimerization interface. In some embodiments, the at least one dimerization interface is capable of forming at least one disulfide bond. In some embodiments, the protein forms a dimer upon expression ex vivo, in vivo, or in vitro. In some embodiments, the dimer is a heterodimer. In some embodiments, the heterodimer is stabilized by at least 1, 2, 3, 4, 5, or 10 disulfide bonds. In some embodiments, the protein constructs include a sequence with at least 80% identity to any one of SEQ ID NOs: 1-12. In some embodiments, the protein construct further includes a VH domain and a VL domain with at least 80% identity to any one of SEQ ID NOS: 83-96. In some embodiments, dimerization stabilizes the heavy and the light chain regions of the Fv domain. In some embodiments, the protein is expressed in a cell, tissue, and/or organ. In some embodiments, the protein is expressed in a mammal, such as a human. In some embodiments, the receptor-specific targeting region is functionalized only upon dimerization of the protein. In some embodiments, the receptor-specific targeting region targets at least one of a CTLA-4 receptor, a TIM-3 receptor, a TIGIT receptor, a LAG-3 receptor, a BTLA receptor, a 2B4 receptor, a CD160 receptor, a CD226 receptor, a KIR2DL4 receptor, a CD244 receptor, a TGF-β receptor, and/or a PD1 receptor. In some embodiments, binding of the receptor-specific targeting region blocks at least one immunoinhibitory pathway. In some embodiments, the intracellular signaling domain is functionalized upon dimerization of the protein. In some embodiments, activation of the intracellular signaling domain promotes cytokine signaling. In some embodiments, at least 1, 2, 3, 4, 5, or 10 cytokines are upregulated upon activation of the intracellular signaling domain. In some embodiments, the protein construct is a chimeric antigen receptor (CAR). In some embodiments, the protein includes an amino acid sequence having at least 90% sequence identity to any one of SEQ ID NOs: 1, 5, 17, and/or 18.

In some embodiments, the at least one disulfide bond is formed between pair(s) of cysteine residues of: VH37 and VL95; VH44 and VL100; VH44 and VL105; VH45 and VL87; VH55 and VL101; VH100 and VL50; VH100b and VL49; VH98 and VL46; and/or VH105 and VL43. In some embodiments, the residue numbering is as determined using the Kabat numbering system.

In some embodiments, the VH and VL amino acid sequences of the anti-TBRII dsFv antibody are numbered using either the sequential, or the Kabat numbering system. The sequential numbering indicates the position of each amino acid residue in the linear sequence of the protein, starting from the N-terminal end. For example, if the amino acid in second position is an alanine, its marking would be “2A.” In contrast, Kabat numbering provides a standardized way to compare and analyse antibody sequences, accommodating variations in CDR length between different antibodies. FIGS. 13A-13B provides a non-limiting example sequence with both sequential and Kabat numbering.

Some embodiments provided herein relate to nucleotide sequences encoding any of the protein constructs described herein. Some embodiments provided herein relate to vectors encoding any of the nucleotide sequences described herein. Some embodiments provided herein relate to cells that include any one of the protein constructs described herein. Some embodiments provided herein relate to cells that include any one of the nucleotide sequences described herein. Some embodiments provided herein relate to cells that include any one of the vectors described herein. In some embodiments, the cell is a T lymphocyte or a cytotoxic T lymphocyte. Some embodiments provided herein relate to compositions that include any of the protein constructs, nucleotide sequences, vectors, or cells described herein.

Some embodiments provided herein relate to methods of treating cancer in a subject. In some embodiments, the methods include administering to the subject a composition that includes a cell described herein. Some embodiments provided herein relate to any of the compositions provided herein for use in treating cancer in a subject.

Some embodiments provided herein relate to methods of treating a disease or disorder in a subject. In some embodiments, the methods include administering to the subject a cell expressing a protein construct. In some embodiments, the protein construct includes a receptor-specific targeting region; a transmembrane domain; an intracellular signaling domain; and an Fv domain including at least one dimerization interface. In some embodiments, the at least one dimerization interface is capable of forming at least one disulfide bonds. In some embodiments, the protein construct is functional as part of a heterodimer. In some embodiments, the heterodimer is stabilized by at least 1, 2, 3, 4, 5, or 10 disulfide bonds. In some embodiments, dimerization stabilizes the heavy and the light chain regions of the Fv domain and functionalizes the protein construct. In some embodiments, the receptor-specific targeting region targets at least one of a CTLA-4 receptor, a TIM-3 receptor, a TIGIT receptor, a LAG-3 receptor, a BTLA receptor, a 2B4 receptor, a CD160 receptor, a CD226 receptor, a KIR2DL4 receptor, a CD244 receptor, a TGF-β receptor, and/or a PD1 receptor. In some embodiments, binding of the receptor-specific targeting region blocks at least one immunoinhibitory pathway. In some embodiments, activation of the intracellular signaling domain promotes cytokine signaling. In some embodiments, the disease or disorder is a cancer. In some embodiments, the cancer is glioblastoma, mesothelioma, pancreatic cancer, gastric cancer, blood cancer, lymphoma, multiple myeloma, leukemia diffuse large B-cell lymphoma (DLBCL), follicular lymphoma, mantle cell lymphoma, multiple myeloma, B-cell acute lymphoblastic leukemia (ALL), Large B-cell lymphoma transformed from follicular lymphoma, High grade B-cell lymphoma, Aggressive B-cell lymphoma not otherwise specified (NOS), lung cancer, ovarian cancer, breast cancer, prostate cancer, liver cancer, kidney cancer, stomach cancer, or colon cancer. Some embodiments provided herein relate to any of the compositions provided herein for use in treating a disease or disorder in a subject.

### BRIEF DESCRIPTION OF THE DRAWINGS

FIGS. 1A-1C depict a nonlimiting example schematic of an antibody protein construct (FIG. 1A) with a heterodimerization domain that was generated from the Fv binding region with the engineering of a strategically placed disulfide bunding (FIG. 1C), without which the domains will not pair (FIG. 1B). FIGS. 1B and 1C further depict exemplary SDS-PAGE staining for an Fv region that does not pair, and does pair, respectively.

FIGS. 2A-2D depict a nonlimiting example schematic of an antibody protein construct (FIG. 2A) with a FAB heterodimerization domain (FIG. 2B) that was generated from the Fv binding region with the engineering of a strategically placed disulfide binding (FIG. 2D), without which the domains will not pair (FIG. 2C). FIGS. 2B-2C further depict SDS-PAGE staining gels for a bound FAB region, an Fv region that does not pair, and does pair, respectively.

FIG. 2E depicts a representative binding curve of a FAB antibody fragment to an immobilized TBR2 receptor, as quantified through ELISA.

FIG. 2F depicts a representative binding curve of an FV antibody fragment to an immobilized TBR2 receptor, as quantified through ELISA.

FIG. 2G depicts a representative binding curve of a disulfide stabilized Fv (“dsFv”) antibody fragment to an immobilized TBR2 receptor, as quantified through ELISA.

FIG. 3A depicts a non-limiting example schematic of a T cell expressing transforming growth factor (TGF)-β receptor (“TBR”) complex that can bind to TGF-β, triggering an immunoinhibitory signaling cascade.

FIG. 3B depicts a non-limiting example schematic of expression of cytokine receptors such as the alpha chain and common gamma chain of TBRII, which, in some embodiments, have no effect when a conventional Fv is expressed at the extracellular domain.

FIG. 3C depicts a non-limiting example schematic of a receptor complex which, in some embodiments, the disulfide stabilized Fv causes the construct to heterodimerize, enabling blocking of TGF-β and triggering of cytokine signaling.

FIGS. 4A-4D depict deconvoluted intact mass spectra of FAB (FIG. 4A), Fv (FIGS. 4B-4C), and dsFv (FIG. 4D) purified proteins.

FIG. 5A depicts a non-limiting example cartoon schematic of a cell-based reporter assay, in which TGFBRI and TGFBRII are expressed on a cell's surface.

FIG. 5B depicts a non-limiting example cartoon schematic of one embodiment of the cell-based reporter assay of FIG. 5A, in which the activator is SMAD4, the promoter is SBE, and the reporter gene encodes an alkaline phosphatase.

FIG. 6A depicts a representative graph of alkaline phosphatase produced over a range of TGF-β concentrations by the cell-based reporter assay shown in FIGS. 5A-5B.

FIG. 6B depicts a representative graph of alkaline phosphate produced by the cell reporter assay shown in FIG. 5B, in a system with 1 ng/mL TGF-β and either cells alone, or cells with any increasing concentration of FAB, or dsFv,

FIG. 7 depicts a representative graph of alkaline phosphate produced by the cell reporter assay shown in FIG. 5B, in a system with 1 ng/ml TGF-β and either cells alone, cells with FAB, cells with Fv, or cells with dsFv,

FIG. 8A depicts a non-limiting example cartoon schematic of a cell-based reporter assay, in which TGFBRI and TGFBRII are expressed on a cell's surface, wherein the co-expression of the anti-TBRII disulfide bond stabilized Fv on the cell surface linked to the signaling chains of a cytokine receptor was expected to prevent TGF-β from engaging with TBRI and TBRII receptor complex to block the alkaline phosphatase reporter generation and secretion.

FIG. 8B depicts a non-limiting example cartoon schematic of one embodiment of the cell-based reporter assay of FIG. 8A, in which the activator is SMAD4, the promoter is SBE, and the reporter gene encodes an alkaline phosphatase.

FIG. 9 depicts a representative graph of the cell based reported assay depicted in FIG. 8B, in which alkaline phosphate production is monitored in cells co-expressing anti-TBRII or non-specific dsFv over an increasing concentration of recombinant TGF-β.

FIG. 10A depicts a non-limiting example cartoon schematic of a cell-based reporter assay, in which an IL-7 receptor is expressed on the cell surface.

FIG. 10B depicts a non-limiting example cartoon schematic of one embodiment of the cell-based reporter assay of FIG. 10A, in which the activator is phosphorylated STAT4, the promoter is STAT5, and the reporter gene encodes an alkaline phosphatase.

FIG. 10C depicts a representative graph of the cell based reported assay depicted in FIGS. 10A-10B.

FIG. 11A depicts a non-limiting example cartoon schematic of a cell-based reporter assay, in which a GM-CSF receptor is expressed on the cell surface.

FIG. 11B depicts a non-limiting example cartoon schematic of one embodiment of the cell-based reporter assay of FIG. 11A, in which the activator is phosphorylated STAT4, the promoter is STAT5, and the reporter gene encodes an alkaline phosphatase.

FIG. 11C depicts a representative graph of the cell based reported assay depicted in FIGS. 11A-11B.

FIG. 12A depicts representative flow cytometry graphs for anti-HA versus FSC-A positive cells in non-transduced (left panel) versus transduced (right panel) cells.

FIG. 12B depicts a representative bar graph of the fold-change in total number of cells compared to the number of cells recovered from the non-transduced (NT) population.

FIG. 13A depicts a non-limiting example schematic of the sequence numbering for an example dsFv VH sequence with both the sequential and the Kabat numbering systems. In this example, the modified cysteine is at position 46 using sequential numbering, and position 44 using Kabat numbering.

FIG. 13B depicts a non-limiting example schematic of the sequence numbering for an example dsFv VL sequence with both the sequential and the Kabat numbering systems. In this example, the modified cysteine is at position 100 using sequential numbering, and position 100 using Kabat numbering.

FIG. 14A depicts a non-limiting cartoon schematic of embodiments of the methods described herein.

FIG. 14B depicts representative flow cytometry graphs for transgene detection of cells exposed to a virus encoding a construct consisting of anti-CD19 CAR only or anti-CD19 CAR and anti-TBRII dsFv with constitutive cytokine receptor or an anti-CD19 CAR and a non-specific dsFv with constitutive cytokine receptor or an anti-CD19 CAR and a anti-TBRII dsFv with truncated cytokine receptor.

FIG. 14C depicts a representative graph for the percent of remaining target cells expressing CD19 or wild-type variants in the presence or absence of recombinant TGF-β cytokine, following exposure to an anti-CD19 CAR.

FIG. 14D depicts a representative graph for the concentration of IFN-γ (pg/mL) in supernatant as measured via an ELISA, in the presence or absence of recombinant TGF-β cytokine, following exposure to an anti-CD19 CAR.

### DETAILED DESCRIPTION

Although the disclosure is described in various exemplary alternatives and implementations as provided herein, it should be understood that the various features, aspects, and functionality described in one or more of the individual alternatives are not limited in their applicability to the particular alternative with which they are described. Instead, they can be applied alone or in various combinations to one or more of the other alternatives of the embodiments described herein, whether the alternatives are described or whether the features are presented as being a part of the described alternative. The breadth and scope of the present disclosure should not be limited by any exemplary alternatives described or shown herein.

Disclosed herein are embodiments of a protein construct expressed on an immune cell that performs the dual function of blocking an immunoinhibitory pathway while providing an independent constitutive cytokine signal. Embodiments of the protein constructs described herein have a number of advantages over similar protein constructs in the field, including, for example: (1) the use of a blocking Fv as the heterodimerization motif, which enables dual function of the module, simplifying a cassette that incorporates the module in an immunotherapy; (2) the Fv blocking/heterodimerization motif is compact, leading to enhanced downstream signaling of the cytokine receptor; and (3) heterodimerization in combination with binding to surface receptors enhances receptor clustering and thus downstream signaling. The embodiments described herein provide further advantages over decoy receptors as the affinity of the binding antibody can be tuned to enhance the blocking capacity to the receptor and thus can be expressed at lower levels than decoy receptors, which require high expression to out-compete the endogenous receptor on the cell surface.

Some embodiments provided herein relate to protein constructs capable of dimerizing. In some embodiments, the protein construct includes a receptor-specific targeting region, a transmembrane domain, an intracellular signaling domain, and an Fv domain including an at least one dimerization interface. In some embodiments, the protein forms a dimer upon expression ex vivo, in vivo, and/or in vitro. In some embodiments, the protein construct forms a polymer when expressed. In some embodiments, the dimer is a heterodimer. In some embodiments, the protein construct forms a tetramer when expressed. In some embodiments, the protein construct forms a heterodimer when expressed. In some embodiments, the heterodimer includes a heterodimeric interface. In some embodiments, the protein construct forms a tetramer consisting of a dimer of heterodimers when expressed. In some embodiments, the tetramer is a homodimer of heterodimers. In some embodiments, the tetramer is a heterodimer of homodimers.

In some embodiments, the at least one dimerization interface is capable of forming at least one molecular bond. In some embodiments, the at least one molecular bond includes a covalent bond. In some embodiments, the at least one molecular bond includes an ionic bond. In some embodiments, the at least one molecular bond includes a disulfide bond. In some embodiments, the at least one molecular bond includes a disulfide bond mimic. In some embodiments, the at least one molecular bond includes a mixture of types of bonds. In some embodiments, the at least one molecular bond includes only a single time of bond. In some embodiments, the at least one molecular bond includes at least 1, 2, 3, 4, 5, 10, 20, 50, or any integer between 1 and 50, molecular bonds. In some embodiments, the at least one molecular bond includes at least 1, 2, 3, 4, 5, 10, 20, 50, or any integer between 1 and 50, disulfide bonds.

In some embodiments, dimerization is driven by the minimal binding region of the Fv domain of an antibody. In some embodiments, the antibody functions in vivo to block an immunoinhibitory pathway (for example TGF-β). In some embodiments, the Fv domain is stabilized. In some embodiments, the Fv domain is stabilized by the presence of an at least one molecular bond between the heavy and light chain. In some embodiments, the Fv domain is stabilized by the presence of at least one covalent bond between the heavy and light chain. In some embodiments, the Fv domain is stabilized by the presence of at least one ionic bond between the heavy and light chain. In some embodiments, the Fv domain is stabilized by the presence of at least one disulfide bond between the heavy and light chain. In some embodiments, the Fv domain is stabilized through at least 1, 2, 3, 4, 5, 10, 20, 50, or any integer between 1 and 50, molecular bonds. In some embodiments, the Fv domain is stabilized through at least 1, 2, 3, 4, 5, 10, 20, 50, or any integer between 1 and 50, disulfide bonds. In some embodiments, the Fv domain is stabilized by at least two types of bonds between the heavy and light chain. In some embodiments, the Fv domain is stabilized by an at least one disulfide mimic bond between the heavy and light chain. In some embodiments, the at least one molecular bond includes a mixture of types of bonds. In some embodiments, the at least one molecular bond includes only a single time of bond. In some embodiments, the heterodimer is stabilized by at least 1, 2, 3, 4, 5, 6, 7, 8, 9, or 10 disulfide bonds. In some embodiments, dimerization stabilizes the heavy and the light chain regions of the Fv domain.

In some embodiments, the protein construct includes a sequence with at least 70%, 75%, 80%, 85%, 90%, 95%, 99%, 100%, or any integer that is between 70% and 100%, identity to any one of SEQ ID NOs: 1-12. In some embodiments, the protein construct includes a sequence with at least 70%, 75%, 80%, 85%, 90%, 95%, 99%, 100%, or any integer that is between 70% and 100%, identity to SEQ ID NO: 1. In some embodiments, the protein construct includes a sequence with at least 70%, 75%, 80%, 85%, 90%, 95%, 99%, 100%, or any integer that is between 70% and 100%, identity to SEQ ID NO: 2. In some embodiments, the protein construct includes a sequence with at least 70%, 75%, 80%, 85%, 90%, 95%, 99%, 100%, or any integer that is between 70% and 100%, identity to SEQ ID NO: 3. In some embodiments, the protein construct includes a sequence with at least 70%, 75%, 80%, 85%, 90%, 95%, 99%, 100%, or any integer that is between 70% and 100%, identity to SEQ ID NO: 4. In some embodiments, the protein construct includes a sequence with at least 70%, 75%, 80%, 85%, 90%, 95%, 99%, 100%, or any integer that is between 70% and 100%, identity to SEQ ID NO: 5. In some embodiments, the protein construct includes a sequence with at least 70%, 75%, 80%, 85%, 90%, 95%, 99%, 100%, or any integer that is between 70% and 100%, identity to SEQ ID NO: 6. In some embodiments, the protein construct includes a sequence with at least 70%, 75%, 80%, 85%, 90%, 95%, 99%, 100%, or any integer that is between 70% and 100%, identity to SEQ ID NO: 7. In some embodiments, the protein construct includes a sequence with at least 70%, 75%, 80%, 85%, 90%, 95%, 99%, 100%, or any integer that is between 70% and 100%, identity to SEQ ID NO: 8. In some embodiments, the protein construct includes a sequence with at least 70%, 75%, 80%, 85%, 90%, 95%, 99%, 100%, or any integer that is between 70% and 100%, identity to SEQ ID NO: 9. In some embodiments, the protein construct includes a sequence with at least 70%, 75%, 80%, 85%, 90%, 95%, 99%, 100%, or any integer that is between 70% and 100%, identity to SEQ ID NO: 10. In some embodiments, the protein construct includes a sequence with at least 70%, 75%, 80%, 85%, 90%, 95%, 99%, 100%, or any integer that is between 70% and 100%, identity to SEQ ID NO: 11. In some embodiments, the protein construct includes a sequence with at least 70%, 75%, 80%, 85%, 90%, 95%, 99%, 100%, or any integer that is between 70% and 100%, identity to SEQ ID NO: 12.

Also disclosed herein is a nucleotide sequence encoding the protein construct of any of the embodiments of the present application. In some embodiments, the nucleotide sequence includes a sequence with at least 70%, 75%, 80%, 85%, 90%, 95%, 99%, 100%, or any integer that is between 70% and 100%, identity to any one of SEQ ID NOs: 13-24. In some embodiments, the nucleotide sequence includes a sequence with at least 70%, 75%, 80%, 85%, 90%, 95%, 99%, 100%, or any integer that is between 70% and 100%, identity to SEQ ID NO: 13. In some embodiments, the nucleotide sequence includes a sequence with at least 70%, 75%, 80%, 85%, 90%, 95%, 99%, 100%, or any integer that is between 70% and 100%, identity to SEQ ID NO: 14. In some embodiments, the nucleotide sequence includes a sequence with at least 70%, 75%, 80%, 85%, 90%, 95%, 99%, 100%, or any integer that is between 70% and 100%, identity to SEQ ID NO: 15. In some embodiments, the nucleotide sequence includes a sequence with at least 70%, 75%, 80%, 85%, 90%, 95%, 99%, 100%, or any integer that is between 70% and 100%, identity to SEQ ID NO: 16. In some embodiments, the nucleotide sequence includes a sequence with at least 70%, 75%, 80%, 85%, 90%, 95%, 99%, 100%, or any integer that is between 70% and 100%, identity to SEQ ID NO: 17. In some embodiments, the nucleotide sequence includes a sequence with at least 70%, 75%, 80%, 85%, 90%, 95%, 99%, 100%, or any integer that is between 70% and 100%, identity to SEQ ID NO: 18. In some embodiments, the nucleotide sequence includes a sequence with at least 70%, 75%, 80%, 85%, 90%, 95%, 99%, 100%, or any integer that is between 70% and 100%, identity to SEQ ID NO: 19. In some embodiments, the nucleotide sequence includes a sequence with at least 70%, 75%, 80%, 85%, 90%, 95%, 99%, 100%, or any integer that is between 70% and 100%, identity to SEQ ID NO: 20 In some embodiments, the nucleotide sequence includes a sequence with at least 70%, 75%, 80%, 85%, 90%, 95%, 99%, 100%, or any integer that is between 70% and 100%, identity to SEQ ID NO: 21. In some embodiments, the nucleotide sequence includes a sequence with at least 70%, 75%, 80%, 85%, 90%, 95%, 99%, 100%, or any integer that is between 70% and 100%, identity to SEQ ID NO: 22. In some embodiments, the nucleotide sequence includes a sequence with at least 70%, 75%, 80%, 85%, 90%, 95%, 99%, 100%, or any integer that is between 70% and 100%, identity to SEQ ID NO: 23. In some embodiments, the nucleotide sequence includes a sequence with at least 70%, 75%, 80%, 85%, 90%, 95%, 99%, 100%, or any integer that is between 70% and 100%, identity to SEQ ID NO: 24.

In some embodiments, at least one disulfide bond is formed between a pair of cysteine residues in the protein construct. In some embodiments, at least one disulfide bond is formed between a pair of cysteine residues at positions VH37 and VL95 of the protein construct. In some embodiments, at least one disulfide bond is formed between a pair of cysteine residues at positions VH44 and VL100 of the protein construct. In some embodiments, at least one disulfide bond is formed between a pair of cysteine residues at positions VH44 and VL105 of the protein construct. In some embodiments, at least one disulfide bond is formed between a pair of cysteine residues at positions VH45 and VL87 of the protein construct. In some embodiments, at least one disulfide bond is formed between a pair of cysteine residues at positions VH55 and VL101 of the protein construct. In some embodiments, at least one disulfide bond is formed between a pair of cysteine residues at positions VH100 and VL50 of the protein construct. In some embodiments, at least one disulfide bond is formed between a pair of cysteine residues at positions VH100b and VL49 of the protein construct. In some embodiments, at least one disulfide bond is formed between a pair of cysteine residues at positions VH98 and VL46 of the protein construct. In some embodiments, at least one disulfide bond is formed between a pair of cysteine residues at positions VH105 and VL43 of the protein construct. The sequences for these modified constructs are as shown in the below Table 1:

TABLE 1
Amino Acid Sequences of disulfide bond constructs, according to Kabat
numbering (SEQ ID NO: 83-96). The modified cysteine is in bold and
underlined for each sequence.

| | SEQ ID | |
|---|---|---|
| Construct: | NO: | Sequence: |
| VH37 | 83 | QLQVQESGPGLVKPSETLSLTCTVSGGSISNSYFSWGWCRQPPGKGLEWIG |
| | | SFYYGEKTYYNPSLKSRATISIDTSKSQFSLKLSSVTAADTAVYYCPRGPTM |
| | | IRGVIDSWGQGTLVTVSS |
| VL95 | 84 | EIVLTQSPATLSLSPGERATLSCRASQSVRSYLAWYQQKPGQAPRLLIYDAS |
| | | NRATGIPARFSGSGSGTDFTLTISSLEPEDFAVYYCQQRSNWCPTFGGGTKV |
| | | EIK |
| VH44 | 85 | QLQVQESGPGLVKPSETLSLTCTVSGGSISNSYFSWGWIRQPPGKCLEWIGS |
| | | FYYGEKTYYNPSLKSRATISIDTSKSQFSLKLSSVTAADTAVYYCPRGPTMI |
| | | RGVIDSWGQGTLVTVSS |
| VL105 | 86 | EIVLTQSPATLSLSPGERATLSCRASQSVRSYLAWYQQKPGQAPRLLIYDAS |
| | | NRATGIPARFSGSGSGTDFTLTISSLEPEDFAVYYCQQRSNWPPTFGGGTKV |
| | | CIK |
| VH45 | 87 | QLQVQESGPGLVKPSETLSLTCTVSGGSISNSYFSWGWIRQPPGKGCEWIGS |
| | | FYYGEKTYYNPSLKSRATISIDTSKSQFSLKLSSVTAADTAVYYCPRGPTMI |
| | | RGVIDSWGQGTLVTVSS |
| VL87 | 88 | EIVLTQSPATLSLSPGERATLSCRASQSVRSYLAWYQQKPGQAPRLLIYDAS |
| | | NRATGIPARFSGSGSGTDFTLTISSLEPEDFAVYCCQQRSNWPPTFGGGTKV |
| | | EIK |
| VH55 | 89 | QLQVQESGPGLVKPSETLSLTCTVSGGSISNSYFSWGWIRQPPGKGLEWIGS |
| | | FYYGCKTYYNPSLKSRATISIDTSKSQFSLKLSSVTAADTAVYYCPRGPTMI |
| | | RGVIDSWGQGTLVTVSS |
| VL101 | 90 | EIVLTQSPATLSLSPGERATLSCRASQSVRSYLAWYQQKPGQAPRLLIYDAS |
| | | NRATGIPARFSGSGSGTDFTLTISSLEPEDFAVYYCQQRSNWPPTFGGCTKV |
| | | EIK |
| VH100 | 91 | QLQVQESGPGLVKPSETLSLTCTVSGGSISNSYFSWGWIRQPPGKGLEWIGS |
| | | FYYGEKTYYNPSLKSRATISIDTSKSQFSLKLSSVTAADTAVYYCPRGPTMI |
| | | CGVIDSWGQGTLVTVSS |
| VL50 | 92 | EIVLTQSPATLSLSPGERATLSCRASQSVRSYLAWYQQKPGQAPRLLIYCAS |
| | | NRATGIPARFSGSGSGTDFTLTISSLEPEDFAVYYCQQRSNWPPTFGGGTKV |
| | | EIK |
| VH98 | 93 | QLQVQESGPGLVKPSETLSLTCTVSGGSISNSYFSWGWIRQPPGKGLEWIGS |
| | | FYYGEKTYYNPSLKSRATISIDTSKSQFSLKLSSVTAADTAVYYCPRGPTCI |
| | | RGVIDSWGQGTLVTVSS |
| VL46 | 94 | EIVLTQSPATLSLSPGERATLSCRASQSVRSYLAWYQQKPGQAPRCLIYDAS |
| | | NRATGIPARFSGSGSGTDFTLTISSLEPEDFAVYYCQQRSNWPPTFGGGTKV |
| | | EIK |
| VH105 | 95 | QLQVQESGPGLVKPSETLSLTCTVSGGSISNSYFSWGWIRQPPGKGLEWIGS |
| | | FYYGEKTYYNPSLKSRATISIDTSKSQFSLKLSSVTAADTAVYYCPRGPTMI |
| | | RGVIDSWGCGTLVTVSS |
| VL43 | 96 | EIVLTQSPATLSLSPGERATLSCRASQSVRSYLAWYQQKPGQCPRLLIYDAS |
| | | NRATGIPARFSGSGSGTDFTLTISSLEPEDFAVYYCQQRSNWPPTFGGGTKV |
| | | EIK |

In some embodiments, the protein construct includes a sequence with at least 70%, 75%, 80%, 85%, 90%, 95%, 99%, 100%, or any integer that is between 70% and 100%, identity to any one of SEQ ID NOs: 25-51. In some embodiments, the protein construct includes a sequence with at least 70%, 75%, 80%, 85%, 90%, 95%, 99%, 100%, or any integer that is between 70% and 100%, identity to SEQ ID NO: 25. In some embodiments, the protein construct includes a sequence with at least 70%, 75%, 80%, 85%, 90%, 95%, 99%, 100%, or any integer that is between 70% and 100%, identity to SEQ ID NO: 26. In some embodiments, the protein construct includes a sequence with at least 70%, 75%, 80%, 85%, 90%, 95%, 99%, 100%, or any integer that is between 70% and 100%, identity to SEQ ID NO: 27. In some embodiments, the protein construct includes a sequence with at least 70%, 75%, 80%, 85%, 90%, 95%, 99%, 100%, or any integer that is between 70% and 100%, identity to SEQ ID NO: 28. In some embodiments, the protein construct includes a sequence with at least 70%, 75%, 80%, 85%, 90%, 95%, 99%, 100%, or any integer that is between 70% and 100%, identity to SEQ ID NO: 29. In some embodiments, the protein construct includes a sequence with at least 70%, 75%, 80%, 85%, 90%, 95%, 99%, 100%, or any integer that is between 70% and 100%, identity to SEQ ID NO: 30. In some embodiments, the protein construct includes a sequence with at least 70%, 75%, 80%, 85%, 90%, 95%, 99%, 100%, or any integer that is between 70% and 100%, identity to SEQ ID NO: 31. In some embodiments, the protein construct includes a sequence with at least 70%, 75%, 80%, 85%, 90%, 95%, 99%, 100%, or any integer that is between 70% and 100%, identity to SEQ ID NO: 32. In some embodiments, the protein construct includes a sequence with at least 70%, 75%, 80%, 85%, 90%, 95%, 99%, 100%, or any integer that is between 70% and 100%, identity to SEQ ID NO: 33. In some embodiments, the protein construct includes a sequence with at least 70%, 75%, 80%, 85%, 90%, 95%, 99%, 100%, or any integer that is between 70% and 100%, identity to SEQ ID NO: 34. In some embodiments, the protein construct includes a sequence with at least 70%, 75%, 80%, 85%, 90%, 95%, 99%, 100%, or any integer that is between 70% and 100%, identity to SEQ ID NO: 34. In some embodiments, the protein construct includes a sequence with at least 70%, 75%, 80%, 85%, 90%, 95%, 99%, 100%, or any integer that is between 70% and 100%, identity to SEQ ID NO: 36. In some embodiments, the protein construct includes a sequence with at least 70%, 75%, 80%, 85%, 90%, 95%, 99%, 100%, or any integer that is between 70% and 100%, identity to SEQ ID NO: 37. In some embodiments, the protein construct includes a sequence with at least 70%, 75%, 80%, 85%, 90%, 95%, 99%, 100%, or any integer that is between 70% and 100%, identity to SEQ ID NO: 38. In some embodiments, the protein construct includes a sequence with at least 70%, 75%, 80%, 85%, 90%, 95%, 99%, 100%, or any integer that is between 70% and 100%, identity to SEQ ID NO: 39. In some embodiments, the protein construct includes a sequence with at least 70%, 75%, 80%, 85%, 90%, 95%, 99%, 100%, or any integer that is between 70% and 100%, identity to SEQ ID NO: 40. In some embodiments, the protein construct includes a sequence with at least 70%, 75%, 80%, 85%, 90%, 95%, 99%, 100%, or any integer that is between 70% and 100%, identity to SEQ ID NO: 41. In some embodiments, the protein construct includes a sequence with at least 70%, 75%, 80%, 85%, 90%, 95%, 99%, 100%, or any integer that is between 70% and 100%, identity to SEQ ID NO: 42. In some embodiments, the protein construct includes a sequence with at least 70%, 75%, 80%, 85%, 90%, 95%, 99%, 100%, or any integer that is between 70% and 100%, identity to SEQ ID NO: 43. In some embodiments, the protein construct includes a sequence with at least 70%, 75%, 80%, 85%, 90%, 95%, 99%, 100%, or any integer that is between 70% and 100%, identity to SEQ ID NO: 44. In some embodiments, the protein construct includes a sequence with at least 70%, 75%, 80%, 85%, 90%, 95%, 99%, 100%, or any integer that is between 70% and 100%, identity to SEQ ID NO: 45. In some embodiments, the protein construct includes a sequence with at least 70%, 75%, 80%, 85%, 90%, 95%, 99%, 100%, or any integer that is between 70% and 100%, identity to SEQ ID NO: 46. In some embodiments, the protein construct includes a sequence with at least 70%, 75%, 80%, 85%, 90%, 95%, 99%, 100%, or any integer that is between 70% and 100%, identity to SEQ ID NO: 47. In some embodiments, the protein construct includes a sequence with at least 70%, 75%, 80%, 85%, 90%, 95%, 99%, 100%, or any integer that is between 70% and 100%, identity to SEQ ID NO: 48. In some embodiments, the protein construct includes a sequence with at least 70%, 75%, 80%, 85%, 90%, 95%, 99%, 100%, or any integer that is between 70% and 100%, identity to SEQ ID NO: 49. In some embodiments, the protein construct includes a sequence with at least 70%, 75%, 80%, 85%, 90%, 95%, 99%, 100%, or any integer that is between 70% and 100%, identity to SEQ ID NO: 50. In some embodiments, the protein construct includes a sequence with at least 70%, 75%, 80%, 85%, 90%, 95%, 99%, 100%, or any integer that is between 70% and 100%, identity to SEQ ID NO: 51.

Also disclosed herein is a nucleotide encoding any one of the protein constructs of any of the embodiments of the present application. In some embodiments, the nucleotide sequence includes a sequence with at least 70%, 75%, 80%, 85%, 90%, 95%, 99%, 100%, or any integer that is between 70% and 100%, identity to any one of SEQ ID NOs: 52-78. In some embodiments, the nucleotide sequence includes a sequence with at least 70%, 75%, 80%, 85%, 90%, 95%, 99%, 100%, or any integer that is between 70% and 100%, identity to SEQ ID NO: 52. In some embodiments, the nucleotide sequence includes a sequence with at least 70%, 75%, 80%, 85%, 90%, 95%, 99%, 100%, or any integer that is between 70% and 100%, identity to SEQ ID NO: 53. In some embodiments, the nucleotide sequence includes a sequence with at least 70%, 75%, 80%, 85%, 90%, 95%, 99%, 100%, or any integer that is between 70% and 100%, identity to SEQ ID NO: 54. In some embodiments, the nucleotide sequence includes a sequence with at least 70%, 75%, 80%, 85%, 90%, 95%, 99%, 100%, or any integer that is between 70% and 100%, identity to SEQ ID NO: 55. In some embodiments, the nucleotide sequence includes a sequence with at least 70%, 75%, 80%, 85%, 90%, 95%, 99%, 100%, or any integer that is between 70% and 100%, identity to SEQ ID NO: 56. In some embodiments, the nucleotide sequence includes a sequence with at least 70%, 75%, 80%, 85%, 90%, 95%, 99%, 100%, or any integer that is between 70% and 100%, identity to SEQ ID NO: 57. In some embodiments, the nucleotide sequence includes a sequence with at least 70%, 75%, 80%, 85%, 90%, 95%, 99%, 100%, or any integer that is between 70% and 100%, identity to SEQ ID NO: 58. In some embodiments, the nucleotide sequence includes a sequence with at least 70%, 75%, 80%, 85%, 90%, 95%, 99%, 100%, or any integer that is between 70% and 100%, identity to SEQ ID NO: 59. In some embodiments, the nucleotide sequence includes a sequence with at least 70%, 75%, 80%, 85%, 90%, 95%, 99%, 100%, or any integer that is between 70% and 100%, identity to SEQ ID NO: 60. In some embodiments, the nucleotide sequence includes a sequence with at least 70%, 75%, 80%, 85%, 90%, 95%, 99%, 100%, or any integer that is between 70% and 100%, identity to SEQ ID NO: 61. In some embodiments, the nucleotide sequence includes a sequence with at least 70%, 75%, 80%, 85%, 90%, 95%, 99%, 100%, or any integer that is between 70% and 100%, identity to SEQ ID NO: 62 In some embodiments, the nucleotide sequence includes a sequence with at least 70%, 75%, 80%, 85%, 90%, 95%, 99%, 100%, or any integer that is between 70% and 100%, identity to SEQ ID NO: 63. In some embodiments, the nucleotide sequence includes a sequence with at least 70%, 75%, 80%, 85%, 90%, 95%, 99%, 100%, or any integer that is between 70% and 100%, identity to SEQ ID NO: 64. In some embodiments, the nucleotide sequence includes a sequence with at least 70%, 75%, 80%, 85%, 90%, 95%, 99%, 100%, or any integer that is between 70% and 100%, identity to SEQ ID NO: 65. In some embodiments, the nucleotide sequence includes a sequence with at least 70%, 75%, 80%, 85%, 90%, 95%, 99%, 100%, or any integer that is between 70% and 100%, identity to SEQ ID NO: 66. In some embodiments, the nucleotide sequence includes a sequence with at least 70%, 75%, 80%, 85%, 90%, 95%, 99%, 100%, or any integer that is between 70% and 100%, identity to SEQ ID NO: 67. In some embodiments, the nucleotide sequence includes a sequence with at least 70%, 75%, 80%, 85%, 90%, 95%, 99%, 100%, or any integer that is between 70% and 100%, identity to SEQ ID NO: 68. In some embodiments, the nucleotide sequence includes a sequence with at least 70%, 75%, 80%, 85%, 90%, 95%, 99%, 100%, or any integer that is between 70% and 100%, identity to SEQ ID NO: 59. In some embodiments, the nucleotide sequence includes a sequence with at least 70%, 75%, 80%, 85%, 90%, 95%, 99%, 100%, or any integer that is between 70% and 100%, identity to SEQ ID NO: 70. In some embodiments, the nucleotide sequence includes a sequence with at least 70%, 75%, 80%, 85%, 90%, 95%, 99%, 100%, or any integer that is between 70% and 100%, identity to SEQ ID NO: 71. In some embodiments, the nucleotide sequence includes a sequence with at least 70%, 75%, 80%, 85%, 90%, 95%, 99%, 100%, or any integer that is between 70% and 100%, identity to SEQ ID NO: 72. In some embodiments, the nucleotide sequence includes a sequence with at least 70%, 75%, 80%, 85%, 90%, 95%, 99%, 100%, or any integer that is between 70% and 100%, identity to SEQ ID NO: 73. In some embodiments, the nucleotide sequence includes a sequence with at least 70%, 75%, 80%, 85%, 90%, 95%, 99%, 100%, or any integer that is between 70% and 100%, identity to SEQ ID NO: 74. In some embodiments, the nucleotide sequence includes a sequence with at least 70%, 75%, 80%, 85%, 90%, 95%, 99%, 100%, or any integer that is between 70% and 100%, identity to SEQ ID NO: 75. In some embodiments, the nucleotide sequence includes a sequence with at least 70%, 75%, 80%, 85%, 90%, 95%, 99%, 100%, or any integer that is between 70% and 100%, identity to SEQ ID NO: 76. In some embodiments, the nucleotide sequence includes a sequence with at least 70%, 75%, 80%, 85%, 90%, 95%, 99%, 100%, or any integer that is between 70% and 100%, identity to SEQ ID NO: 77. In some embodiments, the nucleotide sequence includes a sequence with at least 70%, 75%, 80%, 85%, 90%, 95%, 99%, 100%, or any integer that is between 70% and 100%, identity to SEQ ID NO: 78.

Also disclosed herein is a cell expressing the protein construct of any of the embodiments of the present application.

In some embodiments, the protein construct is expressed in a cell, tissue, and/or organ. In some embodiments, the protein construct is expressed in an immune cell. In some embodiments, the protein construct is expressed in an organ system. In some embodiments, the protein construct is expressed in a mammal. In some embodiments, the protein construct is expressed in a human or human cell, human tissue, and/or human organ.

In some embodiments, the receptor-specific targeting region is functionalized only upon dimerization of the protein. In some embodiments, the receptor-specific targeting region targets at least one of a CTLA-4 receptor, a TIM-3 receptor, a TIGIT receptor, a LAG-3 receptor, a BTLA receptor, a 2B4 receptor, a CD160 receptor, a CD226 receptor, a KIR2DL4 receptor, a CD244 receptor, a TGF-β receptor, and/or a PD1 receptor. In some embodiments, binding of the receptor-specific targeting region blocks at least one immunoinhibitory pathway.

In some embodiments, the intracellular signaling domain is functionalized upon dimerization of the protein. In some embodiments, activation of the intracellular signaling domain promotes cytokine signaling. In some embodiments, at least 1, 2, 3, 4, 5, 6, 7, 8, 9, or 10 cytokines are upregulated upon activation of the intracellular signaling domain. In some embodiments, the protein construct is a chimeric antigen receptor (CAR). In some embodiments, the protein includes an amino acid sequence having at least 80%, 85%, 90%, 95%, 99%, 100%, or any integer that is between 80% and 100%, sequence identity to SEQ ID NO: 1.

Some embodiments provided herein relate to nucleotide sequences encoding a protein construct of any one of the embodiments of the present disclosure. In some embodiments is a vector encoding the nucleotide sequence of any one of the embodiments of the present disclosure, and/or encoding the protein construct of any one of the embodiments of the present disclosure.

Some embodiments provided herein relate to a cell including the protein construct of any one of the embodiments of the present disclosure, the nucleotide sequence of any one of the embodiments of the present disclosure, and/or the vector of any one of the embodiments of the present disclosure. In some embodiments, the cell expresses the protein construct of any one of the embodiments of the present disclosure. In some embodiments, the protein construct dimerizes upon expression in the cell. In some embodiments, the protein construct is incorporated into the plasma membrane/cell surface of the cell. In some embodiments, the cell is an immune cell. In some embodiments, the cell is a T lymphocyte or a cytotoxic T lymphocyte. Some embodiments provided herein relate to a composition that includes any of the protein constructs, nucleotide sequences, vectors, and/or cells as described herein.

Some embodiments provided herein relate to methods of treating a patient in need thereof. In some embodiments, the methods include administering to the patient a cell of any one of the embodiments of the present disclosure. In some embodiments, the methods further include administering one or more chemotherapy agents. In some embodiments, the method is used as part of CAR T cell therapy. Some embodiments provided herein relate to any of the compositions provided herein for use in treating a patient in need thereof.

Some embodiments provided herein relate to methods of treating cancer in a patient. In some embodiments, the methods include administering to the patient a cell of any one of the embodiments of the present disclosure. In some embodiments, the method further includes administering one or more chemotherapy agents. In some embodiments, the method is used as part of CAR T cell therapy. Non-limiting examples of cancer include glioblastoma, mesothelioma, gastric cancer, blood cancers, lymphomas, multiple myeloma, leukemia diffuse large B-cell lymphoma (DLBCL), follicular lymphoma, mantle cell lymphoma, multiple myeloma, B-cell acute lymphoblastic leukemia (ALL), Large B-cell lymphoma transformed from follicular lymphoma, High grade B-cell lymphoma, Aggressive B-cell lymphoma not otherwise specified (NOS), lung cancer, ovarian cancer, breast cancer, prostate cancer, liver cancer, kidney cancer, stomach cancer, pancreatic cancer, and colon cancer. Some embodiments provided herein relate to any of the compositions provided herein for use in treating cancer in a subject.

Some embodiments provided herein relate to methods of treating cancer in a patient. In some embodiments, the methods include administering to the patient a cell expressing a protein construct. In some embodiments, the protein construct includes a receptor-specific targeting region; a transmembrane domain; an intracellular signaling domain; and an Fv domain. In some embodiments, the Fv domain includes at least one dimerization interface. In some embodiments, the at least one dimerization interface is capable of forming at least one disulfide bond. In some embodiments, the protein construct is functional as part of a heterodimer. In some embodiments, the methods further include administering one or more chemotherapy agents. In some embodiments, the method is used as part of CAR T cell therapy. Non-limiting examples of cancer include blood cancers, lymphomas, multiple myeloma, leukemia diffuse large B-cell lymphoma (DLBCL), follicular lymphoma, mantle cell lymphoma, multiple myeloma, B-cell acute lymphoblastic leukemia (ALL), Large B-cell lymphoma transformed from follicular lymphoma, High grade B-cell lymphoma, Aggressive B-cell lymphoma not otherwise specified (NOS), lung cancer, ovarian cancer, breast cancer, prostate cancer, liver cancer, kidney cancer, stomach cancer, pancreatic cancer, and colon cancer.

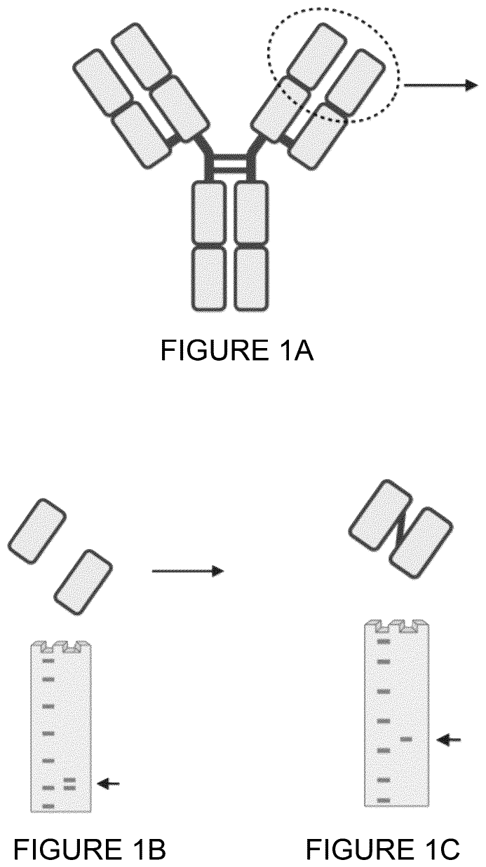

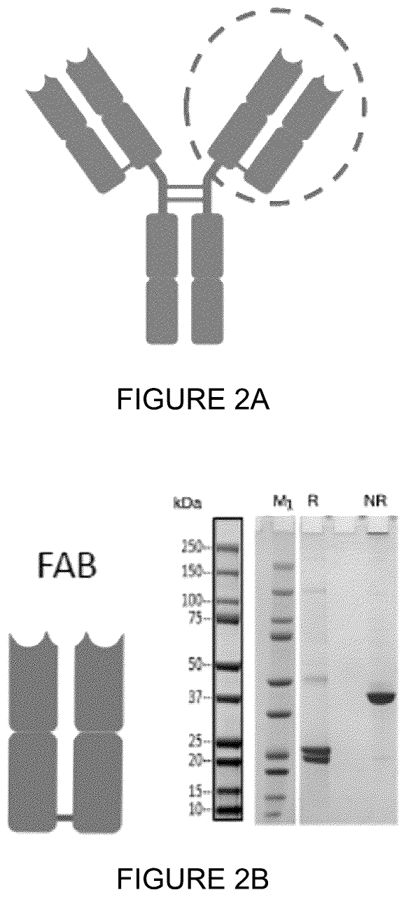

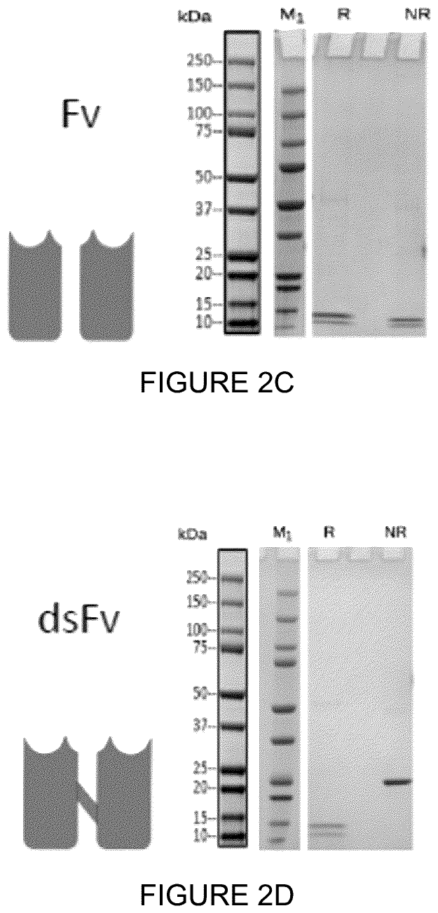

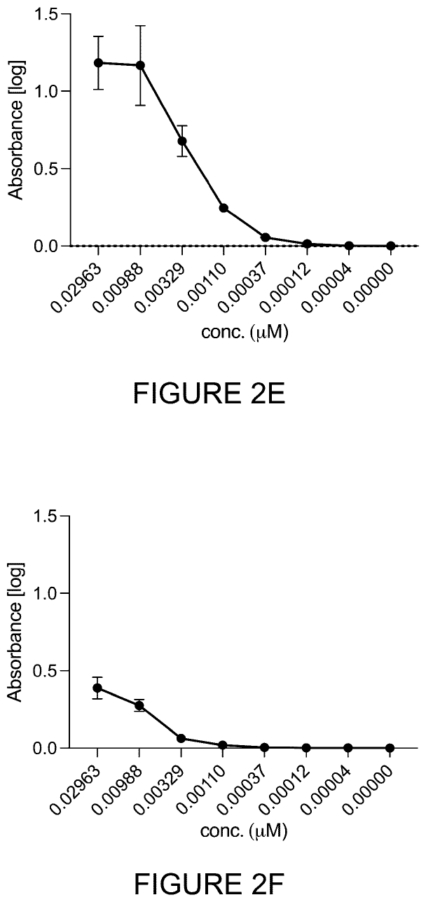

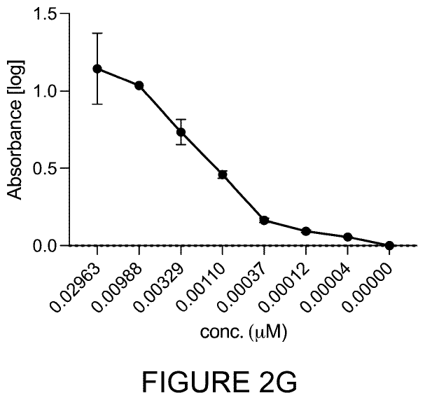

In some embodiments, the functional molecule or protein construct of any one of the embodiments herein cannot be expressed without the stabilization of the Fv domain by the presence of a disulfide bond between the heavy and light chain (FIGS. 1A-1C and 2A-2D). As shown in FIGS. 1A-1C and 2A-2D, if the functional molecule and/or antibody (FIGS. 1A and 2A) is expressed in a cell, purified, and then ran on a protein gel, one of two scenarios would occur: (1) If the molecule and/or antibody is not stabilized by the presence of an at least one additional bond, the protein gel would show two separate bands for the heavy and light chain indicating that the heavy and light chain are destabilized and unlinked (FIGS. 1B and 2C). (2) If, however, the molecule and/or antibody is stabilized by the presence of an at least one additional bond, the heavy and light chain interaction would be stabilized and remain intact, and the protein gel would a single band of a larger molecular weight for the heavy and light chain together (FIGS. 1C and 2D).

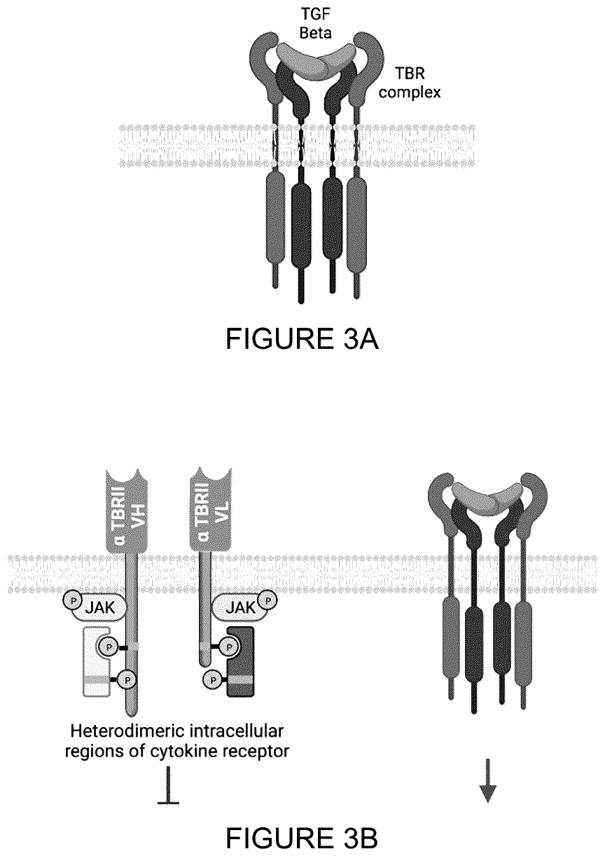

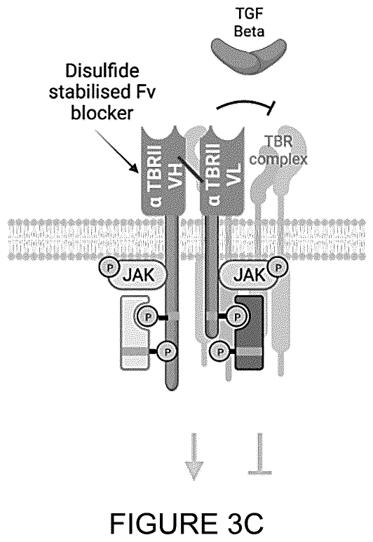

In some embodiments, the stabilization of the Fv heavy and light chains by the disulfide bond enable dimerization of the intracellular regions of cytokine receptor chains. In some embodiments, the dimerization of the intracellular regions of cytokine receptor chains generates a constitutive cytokine signal (FIGS. 3A-3C).

FIG. 3B depicts a non-limiting example schematic of expression of cytokine receptors such as the alpha chain and common gamma chain of TBRII, which, in some embodiments, have no effect when a conventional Fv is expressed at the extracellular domain.

FIG. 3C depicts a non-limiting example schematic of a receptor complex which, in some embodiments, the disulfide stabilized Fv causes the construct to heterodimerize, enabling blocking of TGF-β and triggering of cytokine signaling.

In some embodiments, the protein construct includes a binding domain targeting a receptor. In some embodiments, the protein construct includes a binding domain targeting a TGF-β receptor. In some embodiments, the protein construct includes a binding domain targeting a PD1 receptor. In some embodiments, the protein construct includes a binding domain targeting a CTLA-4 receptor. In some embodiments, the protein construct includes a binding domain targeting a TIM-3 receptor. In some embodiments, the protein construct includes a binding domain targeting a TIGIT receptor. In some embodiments, the protein construct includes a binding domain targeting a LAG-3 receptor. In some embodiments, the protein construct includes a binding domain targeting a BTLA receptor. In some embodiments, the protein construct includes a binding domain targeting a 2B4 receptor. In some embodiments, the protein construct includes a binding domain targeting a CD160 receptor. In some embodiments, the protein construct includes a binding domain targeting a CD226 receptor. In some embodiments, the protein construct includes a binding domain targeting a KIR2DL4 receptor. In some embodiments, the protein construct includes a binding domain targeting a CD244 receptor. In some embodiments, the protein construct includes a binding domain targeting more than one receptor.

In some embodiments, the protein construct includes an intracellular signaling cytokine receptor. In some embodiments, the protein construct includes an IL-7 receptor. In some embodiments, the protein construct includes an GMCSF receptor. In some embodiments, the protein construct includes an IL-2 receptor. In some embodiments, the protein construct includes at least two intracellular signaling cytokine receptors. In some embodiments, the protein construct is capable of eliciting at least one cytokine signal. In some embodiments, the at least one cytokine signal is constitutive. In some embodiments, the dimerization of the protein construct leads to an enhanced downstream signaling of the cytokine receptor.

In some embodiments, the protein construct blocks at least one immunoinhibitory pathway. In some embodiments, the protein construct blocks the PD1 inhibitor pathway. In some embodiments, the protein construct is a PD-L1 or PD-L2 (programmed death ligand 1 or 2). In some embodiments, the protein construct blocks the TGF-β inhibitory pathway. In some embodiments, the protein construct blocks the CTLA-4 inhibitory pathway. In some embodiments, the protein construct is a CD80 (B7-1) or a CD86 (B7-2) ligand. In some embodiments, the protein construct blocks the TIM-3 inhibitory pathway. In some embodiments, the protein construct is a Galectin-9, a carcinoembryonic antigen-related cell adhesion molecule 1 (CEACAM1), or a high-mobility group box 1 (HMGB1) ligand. In some embodiments, the protein construct blocks the TIGIT inhibitory pathway. In some embodiments, the protein construct is a CD155 (poliovirus receptor) or a CD112 (nectin-2) ligand. In some embodiments, the protein construct blocks the LAG-3 inhibitory pathway. In some embodiments, the protein construct is an MHC class II molecule. In some embodiments, the protein construct blocks the BTLA inhibitory pathway. In some embodiments, the protein construct is a herpesvirus entry mediator ligand (HVEM). In some embodiments, the protein construct blocks the 2B4 inhibitory pathway. In some embodiments, the protein construct is a CD48 (BCM1) ligand. In some embodiments, the protein construct blocks the CD160 inhibitory pathway. In some embodiments, the protein construct blocks the CD226 inhibitory pathway. In some embodiments, the protein construct is a CD155 (poliovirus receptor) or a CD112 (nectin-2) ligand. In some embodiments, the protein construct blocks the KIR2DL4 inhibitory pathway. In some embodiments, the protein construct is an HLA-G ligand. In some embodiments, the protein construct blocks the CD244 inhibitory pathway. In some embodiments, the dimerization of the protein construct leads to an inhibition of at least one immunoinhibitory pathway. In some embodiments, the affinity of the binding domain is modulated to improve the capacity of the protein construct to inhibit the at least one immunoinhibitory pathway.

In some embodiments, any one of the modifications to the protein construct is tuned to provide the optimal response both in terms of increased proliferation and ultimately T-cell persistence in addition to potent receptor blocking. In some embodiments, at least two of the modifications to the protein construct are tuned in combination to provide the optimal response both in terms of increased proliferation and ultimately T-cell persistence in addition to potent receptor blocking. Non-limiting examples of the modifications of the protein construct in the present disclosure includes the number of bonds between the heavy and light chain at the dimer interface, the types of bonds between the heavy and light chain at the dimer interface, the area of bonding between the heavy and light chain at the dimer interface, the intracellular signaling cytokine receptor domain, the binding domain targeting a receptor, the affinity of binding domains, and the length of the protein.

The success of adoptive T cell-based immunotherapy, including chimeric antigen receptor (CAR) T cells, in solid tumors will need the improved expansion and cytotoxicity of tumor-directed T-cells within tumor sites. Pro-inflammatory cytokines have been shown to provide some desirable benefits. However, challenges to the approach of delivering these cytokines have constrained their clinical use. To circumvent this limitation, constitutively signaling cytokine receptors have been proposed that can trigger immunomodulatory cytokine axes independent of extracellular cytokine.

TGF-β is one of the most important regulators in the tumor microenvironment (TME). TGF-β plays a key role in tumor initiation and progression while having an adverse effect on antitumor immune surveillance. It has been reported that TGF-β markedly suppresses the cytotoxic function of CD8+ T-cells through transcriptional downregulation of genes encoding key proteins, such as perforin, granzymes, and cytokines in addition to its effects in and around the TME.

TGF-β elicits immune signaling in a subject through its binding to a TGF-β receptor (FIG. 3A). TGF-β receptors are serine/threonine kinase receptors and are members of one of three TGF-β superfamilies of receptors. Receptors within the TGF-β families have differing affinities for TGF-β as a monomer, homodimer, and heterodimer. TGF-β receptors can also bind a wide variety of growth factor and cytokine signaling proteins within the TGF-beta family, such as TGFβs (TGFβ1, TGFβ2, TGFβ3), bone morphogenetic proteins (BMPs), growth differentiation factors (GDFs), activin and inhibin, myostatin, anti-Müllerian hormone (AMH), and NODAL.

TGF-β receptors each have at least one, and often many, phosphorylation sites on their intracellular portions (FIG. 3B). Phosphorylation of TGF-β receptors is a reversible process, and regulates signaling activity for those receptors. For example, Janus kinases (JAK) proteins may phosphorylate a TGF-β receptor once that receptor binds to its target, such as TGF-β. This newly phosphorylated residue then acts as a binding site for a Signal Transducer and Activator of Transcription (STAT). Once the STAT is bound, it is phosphorylated by JAK and forms a homodimer with another STAT. This complex then dissociates from the receptor, travels to the nucleus, and induces transcription of crucial genes. Given that this process requires the initial formation of a heterodimer between the TGF-β receptor and its target, alterations to the heterodimer would predictably have significant effects on gene expression.

Several studies have looked to reduce the immunoinhibitory effects of TGF-β through expression of a decoy TGF-β receptor II (“TGFBR2”, “TBR2,” or “TBRII”). Expression of such proteins in combination with CAR T-cells can circumvent the effects of TGF-β, reduce induced regulatory T-cell (Treg) conversion and prevent the exhaustion of CAR T-cells. These decoy receptors, also referred to as “pseudoreceptors,” can negatively regulate TGF-β receptor activity in cells. Namely, the decoy TBRII can stably associate with TGF-β family receptors and inhibit their signaling. As disclosed in the present application, the presence of a disulfide bond between the VH and VL domains in TBRII further stabilizes TBRII, and enhances its ability to regulate TGF-β-initiated signaling (FIG. 3C).

The beneficial impacts of improving persistence of CAR T-cells and overcoming the immunoinhibitory TME are well understood. However, a practical limitation of ‘modules’ that address these issues is the size limitation placed on DNA insertion in viral vectors commonly utilized for delivery into T-cells. Thus, a need exists to engineer protein constructs that perform multiple functions necessary for an effective CAR T therapy to be engineered into a single viral vector. Such an approach will enhance activity of the CAR T-cells while enabling manufacturing to proceed using processes that are simple and well established.

The following definitions are provided to facilitate understanding of the alternatives or alternatives of the embodiments described herein. Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed.

As used herein, “a” or “an” may mean one or more.

As used herein, “about” in reference to a numeric value, including, for example, whole numbers, fractions, and percentages, generally refers to a range of numerical values (e.g., +/−5% to 10% of the recited value) that one of ordinary skill in the art would consider equivalent to the recited value (e.g., having the same function or result).

The term “comprising” as used herein is synonymous with “including,” “containing,” or “characterized by,” and is inclusive or open-ended and does not exclude additional, unrecited elements or method steps.

The term “in vitro” as used herein has its ordinary meaning as understood in light of the specification, and refers to a system or condition in a cell, tissue, or organ outside of a subject's body. In some embodiments, the cell, tissue, or organ is not a primary cell, tissue, or organ taken directly from the subject. In some embodiments, the cell is an established cell line. In some embodiments, the cell is derived from a primary cell.

The term “ex vivo” as used herein has its ordinary meaning as understood in light of the specification, and refers to a system or condition in a cell, tissue, or organ outside of a subject's body, which is later returned to the subject's body.

The term “in vivo” as used herein has its ordinary meaning as understood in light of the specification, and refers to a system or condition within a subject's body.

The terms “primary cell,” “primary tissue,” and “primary organ” have their ordinary meaning as understood in light of the specification, and refer to a cell, tissue, or organ, respectively, that has been directly taken from a subject.

As used herein, “nucleic acid” or “nucleic acid molecule” have their ordinary meaning as understood in light of the specification, and refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can include monomers that are naturally occurring nucleotides (such as DNA and RNA), or analogs of naturally occurring nucleotides (e.g., enantiomeric forms of naturally occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids,” which include naturally occurring or modified nucleic acid bases attached to a polyamide backbone. Nucleic acids can be either single stranded or double stranded. In some embodiments, a nucleic acid encoding a chimeric antigen receptor is provided. In some embodiments, a method of making a nucleic acid encoding a chimeric antigen receptor is provided. In some embodiments, a nucleic acid encoding a chimeric antigen receptor specific for a ligand on a B cell is provided. In some embodiments, a nucleic acid encoding a chimeric antigen receptor specific for a ligand on a tumor cell is provided. In some embodiments, the nucleic acid is a DNA encoding a chimeric antigen receptor. In some embodiments, the nucleic acid is an mRNA encoding a chimeric antigen receptor. In some embodiments, the chimeric antigen receptor is bi-specific.

“Vector” as described herein, has its ordinary meaning as understood in light of the specification, and is a nucleic acid vehicle that carries a generic material encoding a protein or mRNA of interest into another cell, such that it is replicated and/or expressed in the cell. There are several types of vectors. Without being limiting, a vector can be a plasmid, viral vector, cosmid, artificial chromosome, or an mRNA. The vector can be linear or circular. In some embodiments provided herein, a viral vector is used to carry the nucleic acid encoding a chimeric antigen receptor. In some embodiments, the viral vector is a lentiviral vector. In some embodiments, the viral vector is a retroviral vector. In some embodiments, the viral vector is a gammaretroviral vector. In some embodiments, the vector is a foamy viral vector. In some embodiments, the vector is a plasmid. In some embodiments, the vector is an mRNA. In some embodiments, the vector is linear and includes telomeres.

An “expression cassette” as described herein, has its ordinary meaning as understood in light of the specification, and refers to a gene operatively linked to a regulatory sequence. Without being limiting, transduction or transfection of an expression cassette into a cell may result in the successful expression of the gene's encoded protein.

“Plasmid” as described herein, has its ordinary meaning as understood in light of the specification, and is a genetic structure in a cell that can replicate independently of the chromosomes. Without being limiting, the plasmid can be a small circular DNA strand in the cytoplasm of a bacterium or protozoan, or a linear nucleic acid.

“Express” or “expression” as described herein have their ordinary meaning as understood in light of the specification, and thus refer to the presence of a molecule in a living system. For example, “gene expression” refers to the transcription and translation of a DNA gene into first an RNA, and then a protein. Similarly, “protein expression” refers to the synthesis, and subsequent presence, of a protein within a system. It will be therefore understood that if a cell is said to “express” protein A, that cell is capable of producing protein A.

The terms “polypeptide”, “peptide”, “protein,” and “protein construct” are used interchangeably herein to refer to polymers of amino acids of any length. The polymer may be linear, cyclic, or branched, it may include modified amino acids, and it may be interrupted by non-amino acids. The terms also encompass amino acid polymers that have been modified, for example, via sulfation, glycosylation, lipidation, acetylation, phosphorylation, iodination, methylation, oxidation, proteolytic processing, phosphorylation, prenylation, racemization, selenoylation, transfer-RNA mediated addition of amino acids to proteins such as arginylation, ubiquitination, or any other manipulation, such as conjugation with a labeling component. In some embodiments, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification. Standard amino acids can be written in their full name, three letter name, or one letter name; for example: Histidine, His, or H. Non-limiting examples of amino acids include: histidine, lysine, methionine, phenylalanine, threonine, tryptophane, asparagine, aspartic acid/aspartate, alanine, arginine, cysteine, glutamic acid/glutamate, glutamine, glycine, proline, serine, and tyrosine.

As used herein the term “amino acid” has its ordinary meaning as understood in light of the specification refers to either natural and/or unnatural or synthetic amino acids, including glycine and both the D or L optical isomers, and amino acid analogs and peptidomimetics. For purposes of classifying amino acids substitutions as conservative or nonconservative, amino acids are grouped as follows: Group I (hydrophobic side chains): met, ala, val, leu, ile; Group II (neutral hydrophilic side chains): cys, ser, thr; Group III (acidic side chains): asp, glu; Group IV (basic side chains): asn, gln, his, lys, arg; Group V (residues influencing chain orientation): gly, pro; and Group VI (aromatic side chains): trp, tyr, phe. Conservative substitutions involve substitutions between amino acids in the same class. Non-conservative substitutions constitute exchanging a member of one of these classes for a member of another.

A polypeptide or amino acid sequence “derived from” a designated protein refers to the origin of the polypeptide. Preferably, the polypeptide has an amino acid sequence that is essentially identical to that of a polypeptide encoded in the sequence, or a portion thereof wherein the portion consists of at least 10-20 amino acids, or at least 20-30 amino acids, or at least 30-50 amino acids, or which is immunologically identifiable with a polypeptide encoded in the sequence. This terminology also includes a polypeptide expressed from a designated nucleic acid sequence. Peptide sequences having at least 80%, 85%, 90%, 95%, 99%, or 100% homology to any one of the peptide sequences disclosed herein and having the same or similar functional properties are envisioned. The percent homology may be determined according to amino acid substitutions, deletions, or additions between two peptide sequences. Peptide sequences having some percent homology to any one of the peptide sequences disclosed herein may be produced and tested by one skilled in the art through conventional methods. The % homology or % identity of two sequences is well understood in the art and can be calculated by the number of conserved amino acids or nucleotides relative to the length of the sequences.

“Polymer” refers to a series of monomer groups linked together. A polymer is composed of multiple units of a single monomer (a homopolymer) or different monomers (a heteropolymer). High MW polymers are prepared from monomers that include, but are not limited to, acrylates, methacrylates, acrylamides, methacrylamides, styrenes, vinyl-pyridine, vinyl-pyrrolidone and vinyl esters such as vinyl acetate. Additional monomers are useful in high MW polymers. When two different monomers are used, the two monomers are called “comonomers,” meaning that the different monomers are copolymerized to form a single polymer. The polymer can be linear or branched. When the polymer is branched, each polymer chain is referred to as a “polymer arm.” The end of the polymer arm linked to the initiator moiety is the proximal end, and the growing-chain end of the polymer arm is the distal end. On the growing chain-end of the polymer arm, the polymer arm end group can be the radical scavenger, or another group.

A “monomer” refers to a single protein. A “polymer” refers to more than one proteins connected together through an at least one chemical bond. An “interface” refers to the amino acid region(s) that are connected together.

A “dimer” refers to two proteins connected together. A “homodimer” refers to two identical proteins connected together, for example, Protein A-Protein A. A “heterodimer” refers to two distinct proteins connected together, for example Protein A-Protein B.

A “tetramer” refers to four proteins connected together. A “homotetramer” refers to four identical proteins connected together, for example Protein A-Protein A-Protein A-Protein A. A “heterodimer” refers to any combination of four proteins connected together, wherein at least two proteins are distinct; for example, Protein A-Protein B-Protein C-Protein D, or Protein A-Protein A-Protein B-Protein B, or Protein A-Protein B-Protein A-Protein B, or Protein A-Protein B-Protein B-Protein B. A “homodimer of heterodimers,” then, would be an identical pair of heterodimers connected together, for example, Protein A-Protein B-Protein A-Protein B or Protein A-Protein B-Protein B-Protein A. A “heterodimer of homodimers,” would be two distinct homodimers connected together, for example, Protein A-Protein A-Protein B-Protein B.

A “motif” refers to the primary structure of a strand of nucleotides or amino acids. Non-limiting examples of a nucleotide motif include a stem-loop, G-quadruplex, and D-loop. Non-limiting examples of a protein motif include a beta hairpin, a Greek key, omega loop, helix-loop-helix, zinc finger, helix-turn-helix, nest, and niche motif.

A “chemical linker” refers to a chemical moiety that links two groups together, such as a half-life extending moiety and a protein. The linker can be cleavable or non-cleavable. Cleavable linkers can be hydrolyzable, enzymatically cleavable, pH sensitive, photolabile, or disulfide linkers, among others. Other linkers include homobifunctional and heterobifunctional linkers. A “linking group” is a functional group capable of forming a covalent linkage consisting of one or more bonds to a bioactive agent.

The term “reactive group” refers to a group that is capable of reacting with another chemical group to form a covalent bond, for example, is covalently reactive under suitable reaction conditions, and generally represents a point of attachment for another substance. The reactive group may be a moiety, such as maleimide or succinimidyl ester, and may be capable of chemically reacting with a functional group on a different moiety to form a covalent linkage. Reactive groups generally include nucleophiles, electrophiles, and photoactivatable groups.

“Molecular weight” in the context of the polymer can be expressed as either a number average molecular weight, or a weight average molecular weight or a peak molecular weight. Unless otherwise indicated, all references to molecular weight herein refer to the peak molecular weight. These molecular weight determinations, number average (Mn), weight average (Mw) and peak (Mp), can be measured using size exclusion chromatography or other liquid chromatography techniques. Other methods for measuring molecular weight values can also be used, such as the use of end-group analysis or the measurement of colligative properties (e.g., freezing-point depression, boiling-point elevation, or osmotic pressure) to determine number average molecular weight, or the use of light scattering techniques, ultracentrifugation, or viscometry to determine weight average molecular weight. In some embodiments, the molecular weight is measured by SEC-MALS (size exclusion chromatography-multi angle light scattering). In some embodiments, the polymeric reagents are typically polydisperse (for example, number average molecular weight and weight average molecular weight of the polymers are not equal), and can possess low polydispersity values of, for example, less than about 1.5, as judged, for example, by the PDI value derived from the SEC-MALS measurement. In some embodiments, the polydispersities (PDI) are in the range of about 1.4 to about 1.2. In some embodiments the PDI is less than about 1.15, 1.10, 1.05, or 1.03.

Percentage sequence identities are determined with antibody sequences maximally aligned by the Kabat numbering convention for a variable region or EU numbering for a constant region. After alignment, if a subject antibody region (e.g., the entire mature variable region of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, with gaps not counted, multiplied by 100 to convert to percentage. Sequence identities of other sequences can be determined by aligning sequences using algorithms, such as BESTFIT, FASTA, and TFASTA in the Wisconsin Genetics Software Package Release 7.0, Genetics Computer Group, 575 Science Dr., Madison, WI, using default gap parameters, or by inspection, and the best alignment (for example, resulting in the highest percentage of sequence similarity over a comparison window). Percentage of sequence identity is calculated by comparing two optimally aligned sequences over a window of comparison, determining the number of positions at which the identical residues occur in both sequences to yield the number of matched positions, dividing the number of matched positions by the total number of positions in the window of comparison (for example, the window size), and multiplying the result by 100 to yield the percentage of sequence identity.

“Ligand” as described herein, has its ordinary meaning as understood in light of the specification, and refers to a substance that can form a complex with a biomolecule. By way of example and not of limitation, ligands can include substrates, proteins, small molecules, inhibitors, activators, nucleic acids, and neurotransmitters. Binding can occur through intermolecular forces, for example ionic bonds, hydrogen bonds, and van der walls interactions. Ligand binding to a receptor protein can alter the three-dimensional structure and determine its functional state. The strength of binding of a ligand is referred to as the binding affinity and can be determined by direct interactions and solvent effects. A ligand can be bound by a “ligand binding domain.” A ligand binding domain, for example, can refer to a conserved sequence in a structure that can bind a specific ligand or a specific epitope on a protein. The ligand binding domain or ligand binding portion can include an antibody or binding fragment thereof or scFv, a receptor ligand or mutants thereof, peptide, and/or polypeptide affinity molecule or binding partner. Without being limiting, a ligand binding domain can be a specific protein domain or an epitope on a protein that is specific for a ligand or ligands.

“Affinity” has its ordinary meaning as understood in light of the specification, and refers to the strength that two molecules bind to one another. Therefore, something with a low affinity has a weak bond, while something with a high affinity has a strong bond.

“Protein function” has its ordinary meaning as understood in light of the specification, and refers to the activity of a given protein. For example, if a protein is capable of activating a cellular signal, then that activation is its function. Given that, “dual function” refers to a protein that is capable of at least two activities when expressed.

“Block” or “inhibit” has their ordinary meaning as understood in light of the specification, and refer to reducing or alleviating the functional activity of a protein. For example, if a protein is capable of activating a cellular signal, then blocking that function reduces or eliminates the activation of that cellular signal.

“Specific” or “Specificity” has its ordinary meaning as understood in light of the specification, and can refer to the characteristic of a ligand for the binding partner or alternatively, the binding partner for the ligand, and can include complementary shape, charge, and hydrophobic specificity for binding. Specificity for binding can include stereospecificity, regioselectivity and chemoselectivity. In some embodiments, a chimeric antigen receptor is provided, wherein the chimeric antigen receptor is specific for a B-cell ligand. In some embodiments, a chimeric antigen receptor is provided, wherein the chimeric antigen receptor is specific for a tumor cell ligand.

“Constitutive” has its ordinary meaning as understood in light of the specification, and can refer to the characteristic of an activity that does not need to be induced, or is considered “always on” in a cell, tissue, organ, or system. For example, a “constitutive gene” refers to a gene that is expressed continuously in a cell, and a “constitutive signal” refers to a signal in a cell, tissue, or system, that is continuously active.

“Downstream” and “upstream” have their ordinary meaning as understood in light of the specification, and refer to the relation of one entity compared to another. In the case of a sequence, for any given nucleotide or amino acid “N,” an upstream nucleotide or amino acid occurs previously in that sequence. In contrast, a downstream nucleotide or amino acid occurs later in that sequence. In the case of a cellular pathway or function, for event “A,” an upstream event occurs before event “A,” while a downstream event occurs after event “A.” It will therefore be understood that “downstream signaling,” as used in the present specification, refers to signaling that occurs after the function of the referenced protein occurs.

A “regulatory element” has its ordinary meaning as understood in light of the specification, and as described herein, can refer to a regulatory sequence, which is any DNA sequence that is responsible for the regulation of gene expression, such as promoters and operators. The regulatory element can be a segment of a nucleic acid molecule which is capable of increasing or decreasing the expression of specific genes within an organism. In some embodiments described herein, a cell is provided. In some embodiments, the cell includes a first and second chimeric antigen receptor or TcR, wherein the first chimeric antigen receptor is specific for a ligand on a B cell, which promotes the in vivo expansion and activation of an effector cell and, wherein the second chimeric antigen receptor or TcR is specific for a ligand on a tumor. In some embodiments, the first chimeric antigen receptor and/or the second chimeric antigen receptor or TcR are inducibly expressed in said cell. In some embodiments, expression of the first chimeric antigen receptor and/or the second chimeric antigen receptor or TcR is under the control of a regulatory element.

“Transmembrane domain” has its ordinary meaning as understood in light of the specification, and as described herein is an integral protein that can span a cellular membrane.

A “promoter” has its ordinary meaning as understood in light of the specification, and is a nucleotide sequence that directs the transcription of a structural gene. In some embodiments, a promoter is located in the 5′ non-coding region of a gene, proximal to the transcriptional start site of a structural gene. Sequence elements within promoters that function in the initiation of transcription are often characterized by consensus nucleotide sequences. Without being limiting, these promoter elements can include RNA polymerase binding sites, TATA sequences, CAAT sequences, differentiation-specific elements (DSEs; McGehee et al., Mol. Endocrinol. 7:551 (1993); incorporated by reference in its entirety), cyclic AMP response elements (CREs), serum response elements (SREs; Treisman, Seminars in Cancer Biol. 1:47 (1990); incorporated by reference in its entirety), glucocorticoid response elements (GREs), and binding sites for other transcription factors, such as CRE/ATF (O'Reilly et al., J. Biol. Chem. 267:19938 (1992); incorporated by reference in its entirety), AP2 (Ye et al., J. Biol. Chem. 269:25728 (1994); incorporated by reference in its entirety), SP1, cAMP response element binding protein (CREB; Loeken, Gene Expr. 3:253 (1993); incorporated by reference in its entirety) and octamer factors (see, in general, Watson et al., eds., Molecular Biology of the Gene, 4th ed. (The Benjamin/Cummings Publishing Company, Inc. 1987; incorporated by reference in its entirety)), and Lemaigre and Rousseau, Biochem. J. 303:1 (1994); incorporated by reference in its entirety). As used herein, a promoter can be constitutively active, repressible, or inducible. If a promoter is an inducible promoter, then the rate of transcription increases in response to an inducing agent. In contrast, the rate of transcription is not regulated by an inducing agent if the promoter is a constitutive promoter. Repressible promoters are also known. In some embodiments, the nucleic acid includes a promoter sequence. In some embodiments of the chimeric antigen, the chimeric antigen is inducibly expressed in response to an inducing agent. In some embodiments, the TcR is inducibly expressed in response to an inducing agent.

In some embodiments, promoters used herein can be inducible or constitutive promoters. Without being limiting, inducible promoters can include, for example, a tamoxifen inducible promoter, tetracycline inducible promoter, and doxocycline inducible promoter (e.g., tre) promoter. Constitutive promoters can include, for example, SV40, CMV, UBC, EF1alpha, PGK, and CAGG. In some embodiments, the regulatory is a promoter. In some embodiments, the promoter is a tamoxifen inducible promoter, a tetracycline inducible promoter, or a doxocycline inducible promoter (e.g., tre) promoter. In some embodiments provided herein, expression of a chimeric antigen receptor or a TcR on a cell is induced by tamoxifen and/or its metabolites. Metabolites for tamoxifen are active metabolites such as 4-hyroxytamoxifen (afimoxifene) and N-desmethyl-4-hydroxytamoxifen (endoxifen), which can have 30-100 times more affinity with an estrogen receptor than tamoxifen itself. In some embodiments, the tamoxifen metabolites are 4-hyroxytamoxifen (afimoxifene) and/or N-desmethyl-4-hydroxytamoxifen (endoxifen). In some embodiments, vectors are provided wherein the vector has a first promoter for the CAR/TcR and a second promoter for the marker protein.

The term “cell” includes those of prokaryotes and eukaryotes, and may further include bacterial cells, mycobacteria cells, fungal cells, yeast cells, plant cells, insect cells, non-human animal cells, human cells, or cell fusions such as, for example, hybridomas. In some embodiments, the cell is eukaryotic. In some embodiments, the cell is a mammalian cell. In some embodiments, the cell is derived from human, monkey, ape, hamster, rat, or mouse cells. In some embodiments, the cell is human. In some embodiments, the cell is an immune cell. In some embodiments, the cell is a lymphocyte. In some embodiments, the cell is a T cell. In some embodiments, the cell is a tumor infiltrating lymphocyte (TIL) cell, a natural killer (NK) cell, a CD8+ T cell, a CD4+ T cell, a regulatory T cell, or a memory T cell.

An “antibody” as described herein, has its ordinary meaning as understood in light of the specification, and refers to a large Y-shape protein produced by plasma cells that is used by the immune system to identify and neutralize foreign objects such as bacteria and viruses. The antibody protein can include four polypeptide chains; two identical heavy chains and two identical light chains connected by disulfide bonds. Each chain is composed of structural domains called immunoglobulin domains. These domains can contain about 70, 80, 90, 100, 110, 120, 130, 140, 150 amino acids or any number of amino acids in between in a range defined by any two of these values, and are classified into different categories according to their size and function. In some embodiments, the ligand binding domain includes an antibody or binding fragment thereof or scFv, a receptor ligand or mutants thereof, peptide, and/or polypeptide affinity molecule or binding partner. In some embodiments, the ligand binding domain is an antibody fragment, desirably, a binding portion thereof. In some embodiments, the antibody fragment or binding portion thereof present on a CAR is specific for a ligand on a B-cell. In some embodiments, the antibody fragment or binding portion thereof present on a CAR or TcR is specific for a ligand on a tumor cell. In some embodiments, the tumor is not derived from a B-cell related cancer. In some embodiments, the antibody fragment or binding portion thereof present on a CAR is specific for a ligand present on a tumor cell. In some embodiments, the ligand binding domain is an antibody fragment or a binding portion thereof, such as a single chain variable fragment (scFv). In some embodiments, the ligand includes a tumor specific mutation. In some embodiments, the antibody fragment or binding portion thereof present on a CAR includes one or more domains from a humanized antibody, or binding portion thereof.

Specific binding of an antibody to its target antigen(s) means an affinity of at least 10⁶, 10⁷, 10⁸, 10⁹, or 10¹⁰ M⁻¹. Specific binding is detectably higher in magnitude and distinguishable from non-specific binding occurring to at least one unrelated target. Specific binding can be the result of formation of bonds between particular functional groups or particular spatial fit (e.g., lock and key type) whereas nonspecific binding is usually the result of van der Waals forces. Specific binding does not however necessarily imply that an antibody or fusion protein binds one and only one target.

A basic antibody structural unit is a tetramer of subunits. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. This variable region is initially expressed linked to a cleavable signal peptide. The variable region without the signal peptide is sometimes referred to as a mature variable region. Thus, for example, a light chain mature variable region means a light chain variable region without the light chain signal peptide. However, reference to a variable region does not mean that a signal sequence is necessarily present; and in fact, signal sequences are cleaved once the antibodies or fusion proteins have been expressed and secreted. A pair of heavy and light chain variable regions defines a binding region of an antibody. The carboxy-terminal portion of the light and heavy chains respectively defines light and heavy chain constant regions. The heavy chain constant region is primarily responsible for effector function. In IgG antibodies, the heavy chain constant region is divided into CH1, hinge, CH2, and CH3 regions. The CH1 region binds to the light chain constant region by disulfide and noncovalent bonding. The hinge region provides flexibility between the binding and effector regions of an antibody and also provides sites for intermolecular disulfide bonding between the two heavy chain constant regions in a tetramer subunit. The CH2 and CH3 regions are the primary site of effector functions and FcR binding.

Light chains are classified as either kappa or lambda. Heavy chains are classified as gamma, mu, alpha, delta, or epsilon, and define the antibody's isotype as IgG, IgM, IgA, IgD and IgE, respectively. Within light and heavy chains, the variable and constant regions are joined by a “J” segment of about 12 or more amino acids, with the heavy chain also including a “D” segment of about 10 or more amino acids. (See generally, Fundamental Immunology (Paul, W., ed., 2^{nd} ed. Raven Press, N.Y., 1989), Ch. 7) (incorporated by reference in its entirety for all purposes).

The IgG antibodies includes Fc and Fab domains. The Fab fragment can further be divided into the Fv fragment, which includes a heavy and light chain, and is the smallest fragment that still retains the antigen binding site.

The mature variable regions of each light/heavy chain pair form the antibody binding site. Thus, an intact antibody has two binding sites, for example, is divalent. In natural antibodies, the binding sites are the same. However, bispecific antibodies can be made in which the two binding sites are different (see, e.g., Songsivilai S, Lachmann P C. 1990. Bispecific antibody: a tool for diagnosis and treatment of disease. Clin Exp Immunol. 79:315-321; Kostelny S A, Cole M S, Tso J Y. 1992. Formation of bispecific antibody by the use of leucine zippers. J Immunol. 148:1547-1553). The variable regions all exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. From N-terminal to C-terminal, both light and heavy chains include the domains FR1, CDR1, FR2, CDR2, FR3, CDR3 and FR4. For convenience, the variable heavy CDRs can be referred to as CDRH1, CDRH2 and CDRH3; the variable light chain CDRs can be referred to as CDRL1, CDRL2 and CDRL3. The assignment of amino acids to each domain is in accordance with the definitions of Kabat E A, et al. 1987 and 1991. Sequences of Proteins of Immunological Interest (National Institutes of Health, Bethesda, MD) or Chothia C, Lesk A M. 1987. Canonical Structures for the Hypervariable Regions of Immunoglobulins. J Mol Biol 196:901-917; Chothia C, et al. 1989. Conformations of Immunoglobulin Hypervariable Regions. Nature 342:877-883. Kabat also provides a widely used numbering convention (Kabat numbering) in which corresponding residues between different heavy chain variable regions or between different light chain variable regions are assigned the same number. Although Kabat numbering can be used for antibody constant regions, EU numbering is more commonly used, as is the case in this application. Although specific sequences are provided for exemplary antibodies disclosed herein, it will be appreciated that after expression of protein chains one to several amino acids at the amino or carboxy terminus of the light and/or heavy chain, particularly a heavy chain C-terminal lysine residue, may be missing or derivatized in a proportion or all of the molecules.

In addition to entire immunoglobulins (or their recombinant counterparts), immunoglobulin fragments or “binding fragments” including the epitope binding site (e.g., Fab′, F(ab′)₂, single-chain variable fragment (scFv), diabody, minibody, nanobody, single-domain antibody (sdAb), or other fragments) are useful as antibody moieties in the present disclosure. Such antibody fragments may be generated from whole immunoglobulins by ricin, pepsin, papain, or other protease cleavage. Minimal immunoglobulins may be designed utilizing recombinant immunoglobulin techniques. For instance, “Fv” immunoglobulins for use in the present disclosure may be produced by linking a variable light chain region to a variable heavy chain region via a peptide linker (e.g., poly-glycine or another sequence which does not form an alpha helix or beta sheet motif). Nanobodies or single-domain antibodies can also be derived from alternative organisms, such as dromedaries, camels, llamas, alpacas, or sharks. In some embodiments, antibodies can be conjugates, e.g., pegylated antibodies, drug, radioisotope, or toxin conjugates. Monoclonal antibodies directed against a specific epitope, or combination of epitopes, will allow for the targeting and/or depletion of cellular populations expressing the marker. Various techniques can be utilized using monoclonal antibodies to screen for cellular populations expressing the marker(s), and include magnetic separation using antibody-coated magnetic beads, “panning” with antibody attached to a solid matrix (for example, a plate), and flow cytometry.

As known in the art, the term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is that of the EU index as in Kabat. Kabat et al., Sequences of Proteins of Immunological Interest, 5th Ed. Public Health Service, National Institutes of Health, Bethesda, Md., 1991. The Fc region of an immunoglobulin generally includes two constant domains, CH2 and CH3. As is known in the art, an Fc region can be present in dimer or monomeric form.

As known in the art, a “constant region” of an antibody refers to the constant region of the antibody light chain or the constant region of the antibody heavy chain, either alone or in combination.

A “variable region” of an antibody refers to the variable region of the antibody light chain or the variable region of the antibody heavy chain, either alone or in combination. As known in the art, the variable regions of the heavy and light chains each consist of four framework regions (FRs) connected by three complementarity determining regions (CDRs) also known as hypervariable regions, and contribute to the formation of the antigen binding site of antibodies. If variants of a subject variable region are desired, particularly with substitution in amino acid residues outside of a CDR region (for example, in the framework region), appropriate amino acid substitution, preferably, conservative amino acid substitution, can be identified by comparing the subject variable region to the variable regions of other antibodies which contain CDR1 and CDR2 sequences in the same canonical class as the subject variable region (Chothia and Lesk, J Mol Biol 196 (4): 901-917, 1987).

The term “epitope” refers to a site on an antigen to which an antibody or extracellular trap segment binds. An epitope on a protein can be formed from contiguous amino acids or noncontiguous amino acids juxtaposed by tertiary folding of one or more proteins. Epitopes formed from contiguous amino acids (also known as linear epitopes) are typically retained on exposure to denaturing solvents whereas epitopes formed by tertiary folding (also known as conformational epitopes) are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols, in Methods in Molecular Biology, Vol. 66, Glenn E. Morris, Ed. (1996).

Antibodies that recognize the same or overlapping epitopes can be identified in a simple immunoassay showing the ability of one antibody to compete with the binding of another antibody to a target antigen. The epitope of an antibody can also be defined by X-ray crystallography of the antibody (or Fab fragment) bound to its antigen to identify contact residues.

Alternatively, two antibodies have the same epitope if all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Competition between antibodies is determined by an assay in which an antibody under test inhibits specific binding of a reference antibody to a common antigen (see, e.g., Junghans et al., Cancer Res. 50:1495, 1990). A test antibody competes with a reference antibody if an excess of a test antibody (e.g., at least 2×, 5×, 10×, 20×, or 100×) inhibits binding of the reference antibody by at least 50%. In some embodiments the test antibody inhibits binding of the reference antibody by 75%, 90%, or 99% as measured in a competitive binding assay. Antibodies identified by competition assay (competing antibodies) include antibodies binding to the same epitope as the reference antibody and antibodies binding to an adjacent epitope sufficiently proximal to the epitope bound by the reference antibody for steric hindrance to occur.

“Immune cells” as described herein, have their ordinary meaning as understood in light of the specification, and refer to cells that are part of the immune system. In some embodiments, the cell is part of the innate immune system. In some embodiments, the cell is part of the adaptive immune system. Non-limiting example of immune cells include blood cells, bone marrow cells, hematopoietic stem cells, lymphoid progenitor cells, myeloid progenitor cells, B cell progenitors, memory B cells, plasma cells, monocytes, macrophages, dendritic cells, basophils, neutrophils, eosinophils, mast cells, natural killer cells, T cell progenitors, memory T cell, cytotoxic T cells, and helper T cells.

“Effector cells” as described herein, has its ordinary meaning as understood in light of the specification, and refers to a lymphocyte that has been induced to differentiate into another cell type that can be capable of mounting a specific immune response, such as a terminally differentiated leukocyte that performs one or more specific functions. The main effector cells of the immune system, for example, are activated lymphocytes and phagocytes that are involved in destroying pathogens and removing them from the body. The effector cells can include large granular lymphocytes, such as, for example, natural killer cells and cytotoxic T lymphocytes. In some embodiments of the cells provided herein, the cell includes a first and second chimeric antigen receptor, wherein the first chimeric antigen receptor is specific for a ligand on a B cell, which promotes the in vivo expansion and activation of an effector cell and, wherein the second chimeric antigen receptor is specific for a ligand on a tumor. In some embodiments, the cells that undergo expansion and activation are lymphocytes, phagocytes, large granular lymphocytes, natural killer cells and/or cytotoxic T lymphocytes.

“Cancer antigen,” “tumor antigen,” or “tumor marker” has its ordinary meaning as understood in light of the specification, and refers to an antigenic substance that is produced in a tumor cell, which can therefore trigger an immune response in the host. These cancer antigens can be useful as markers for identifying a tumor cell, which will be a potential candidate during treatment or therapy. There are several types of cancer or tumor antigens. There are tumor specific antigens (TSA) which are present only on tumor cells and not on healthy cells, as well as tumor associated antigens (TAA) which are present in tumor cells and also on some normal cells. In some embodiments of the methods and chimeric antigens provided herein, the chimeric antigen receptors are specific for tumor specific antigens. In some embodiments, the chimeric antigen receptors are specific for tumor associated antigens. In some embodiments described herein, the tumor does not originate from a B-cell related cancer. In some embodiments, cells expressing a CAR that is specific for a TAA is further modified by the introduction of a suicide gene to limit the time of the CAR T-cell therapy and to reduce the attack of normal tissues expressing the TAA.

In any of the embodiments provided herein, the cancer antigen is EGFR, HER2, Mesothelin, cancer testis antigens, LICAM, o-acetylated GD2, GD2, neoantigens, Var2, glypican-2 (GPC2), HPV antigens, alphafetoprotein, carcinoembryonic antigen, CA-125, MUC-1, epithelial tumor antigen, abnormal products of ras or p53, EphA2, MAGE-A3, MAGE-A4, MAGE-C2, PRAME, SSX2, adipophilin, AIM2, ALDH1A1, BCLX, B7H3, EpCAM, claudin 18.2, CS274, CPSF, cyclin D1, DKK1, ENAH, EpCAM, EphA3, EZH2, FAP, FGF5, glypican-3, G250, HLA-DOB, Hepsin, ID01, IGF2B3, IL13Ralpha2, Intestinal carboxylesterase, alpha-foetoprotein, kallikrein4, KIF20A, Lengsin, M-CSF, MCSP, mdm-2, Meloe, midkine, MMP-2, MMP-7, MUC1, MUC5AC, p53, PAX5, PBF, PRAME, PSMA, RAGE-1, RGS5, RhoC, RNF43, RUF43, FU2AS, secernin 1, SOX10, STEAP1, survivin, telomerase, TPBG, VEGF, WT1, NY-ESO-1 or ROR1. In some embodiments, the cell surface tumor specific molecule is ROR1. In some embodiments, the cancer antigen is expressed by a tumor. In some embodiments, the tumor is not a B-cell related cancer.

“Chimeric antigen receptors” (CARs), as described herein, has its ordinary meaning as understood in light of the specification, and refers to genetically engineered protein receptors, which can confer specificity onto an immune effector cell, such as for example, a T-cell. Without being limiting, the use of CAR bearing T-cells can promote in vivo expansion and activation. The CARs can also be designed to redirect T-cells to target cells that express specific cell-surface antigens, where they can activate lymphocytes, such as T-cells, upon target recognition. The CARs graft the specificity of a monoclonal antibody or binding fragment thereof or scFv onto a T-cell, with the transfer of their coding sequence facilitated by vectors. In order to use CARs as a therapy for a subject in need, a technique called adoptive cell transfer is used in which T-cells are removed from a subject and modified so that they can express the CARs that are specific for an antigen. The T-cells, which can then recognize and target an antigen, are reintroduced into the patient. In some embodiments, CAR expressing lymphocytes are described, wherein the CAR expressing lymphocyte can be delivered to a subject to target specific cells. A TcR is a molecule on the surface of T lymphocytes or T-cells that can recognize antigens. As described herein, the CAR promotes in vivo expansion and activation of effector cells.

The structure of the CAR can include fusions of single-chain variable fragments (scFv) that are derived from monoclonal antibodies that are attached to transmembrane and cytoplasmic signaling domains. Most CARs can include an extracellular scFv that is linked to an intracellular CD32 domain (first generation CAR). Additionally, the scFv can be linked to a co-stimulatory domain, which can increase their efficacy in the therapy of a subject in need (second generation CAR). When T-cells express this molecule, they can recognize and kill target cells that express a specific antigen targeted by the CAR.

The chimeric antigen receptor can include a binding portion that is specific for a ligand. Without being limiting, the binding portion can include an antibody or binding fragment thereof or scFv, a receptor ligand or mutants thereof, peptide, and/or polypeptide affinity molecule or binding partner. In some embodiments of the first chimeric antigen receptor, the first chimeric antigen receptor includes a binding portion, wherein the binding portion includes an antibody or binding fragment thereof or scFv, a receptor ligand or mutants thereof, peptide, and/or polypeptide affinity molecule or binding partner. In some embodiments, the binding portion is specific for a ligand on a B-cell. In some embodiments of the second chimeric antigen receptor, the second chimeric antigen receptor includes a binding portion, wherein the binding portion includes an antibody or binding fragment thereof or scFv, a receptor ligand or mutants thereof, peptide, and/or polypeptide affinity molecule or binding partner. In some embodiments, the binding portion is specific for a ligand on a tumor cell. In some embodiments, the tumor is not a tumor of a B-cell related cancer.

In some embodiments, a chimeric antigen receptor is provided, wherein the ligand or target molecule is a cell surface molecule that is found on tumor cells and is not substantially found on normal tissues, or restricted in its expression to non-vital normal tissues. In some embodiments, the tumor does not originate from a B-cell related cancer. In some embodiments, the ligand or target molecule is found on a tumor cell as well as on normal tissues. In some embodiments, the cells expressing a CAR that is specific for a ligand on tumor cells and normal tissue further includes a suicide gene to limit the time of therapy and increase their safety profile. Conditional suicide genes may also be applied to the donor T-cells to limit the attack on normal tissue that may express a tumor associated antigen or ligand.

Although humanized antibodies often incorporate all six CDRs (which can be as defined by Kabat) from a mouse antibody, they can also be made with less than all CDRs (e.g., at least 3, 4, or 5 CDRs from a mouse antibody) (e.g., De Pascalis R, Iwahashi M, Tamura M, et al. 2002. Grafting “Abbreviated” Complementary-Determining Regions Containing Specificity-Determining Residues Essential for Ligand Contact to Engineer a Less Immunogenic Humanized Monoclonal Antibody. J Immunol. 169:3076-3084; Vajdos F F, Adams C W, Breece T N, Presta L G, de Vos A M, Sidhu, S S. 2002. Comprehensive functional maps of the antigen-binding site of an anti-ErbB2 antibody obtained with shotgun scanning mutagenesis. J Mol Biol. 320:415-428; Iwahashi M, Milenic D E, Padlan E A, et al. 1999. CDR substitutions of a humanized monoclonal antibody (CC49): Contributions of individual CDRs to antigen binding and immunogenicity. Mol Immunol. 36:1079-1091; Tamura M, Milenic D E, Iwahashi M, et al. 2000. Structural correlates of an anticarcinoma antibody: Identification of specificity-determining regions (SDRs) and development of a minimally immunogenic antibody variant by retention of SDRs only. J Immunol. 164:1432-1441).

A chimeric antibody is an antibody in which the mature variable regions of light and heavy chains of a non-human antibody (e.g., a mouse) are combined with human light and heavy chain constant regions. Such antibodies substantially or entirely retain the binding specificity of the mouse antibody, and are about two-thirds human sequence.

“Signaling domain” as described herein, has its ordinary meaning as understood in light of the specification, and is a domain on a chimeric antigen receptor that can promote cytokine release, in vivo T cell survival and tumor elimination. In some embodiments herein, a signaling domain includes CD28, 4-1BB and/or CD3-zeta cytoplasmic domains.

A “cytokine” as described herein, has its ordinary meaning as understood in light of the specification, and is a small molecule that is secreted by one cell and that has an effect on other cells. Cytokines, sometime considered as “stress proteins,” include chemokines, interferons, interleukins, lymphokines, and tumor necrosis factors. Cytokines are produced by many cells, including macrophages, B lymphocytes, T lymphocytes and mast cells, as well as endothelial cells, fibroblasts, and various stromal cells. A particular cytokine may be produced by more than one type of cell. Non-limiting examples of cytokines include members of the IL-1 family, TNF family, interferons, IL-6 family, IL-10 family, TGF-beta family, and chemokines. Common cytokines include IL-1, IL-2, IL-3, IL-4, IL-5, IL-6, IL-8, IL-9, IL-10, IL-11, IL-12, IL-13, IL-15, IL-16, IL-18, IL-21, IL-33, IFN-alpha, IFN-beta, IFN-gamma, TNF-alpha, and TNF-beta.

“Cytokine signaling” as described herein, has its ordinary meaning as understood in light of the specification, and refers to the process by which a cytokine is recognized by a cytokine receptor on the surface of a cell, and elicits a response. These signals may either be “autocrine” (wherein the same cell both produces the cytokine and responds to it) or “paracrine” (where the cytokine is made by one cell and acts on another). Cytokine receptors are grouped into six major families: class I cytokine receptors, class II cytokine receptors, IL-1 receptors, TNF receptors, tyrosine kinase receptors, and chemokine receptors. Cytokines activate many pathways; for example, the JAK-STAT pathway. In this pathway, JAK proteins phosphorylate a cytokine receptor once that receptor binds to its corresponding cytokine. This newly phosphorylated residue on the cytokine receptor then acts as a binding site for a STAT protein. Once the STAT is bound, it is phosphorylated by JAK and forms a homodimer with another STAT. This complex then dissociates from the receptor, travels to the nucleus, and induces transcription of crucial genes.

### Methods of Treatment

Some embodiments relate to a method of treatment as part of immunotherapy. “Immunotherapy” has its ordinary meaning as understood in light of the specification, and refers to the process of using a subject's immune system to fight a disease. In some embodiments, immunotherapy is used to target a cancer. In some embodiments, the immunotherapy includes activating an immunoinhibitory pathway. “Immunoinhibitory pathway” has its ordinary meaning as understood in light of the specification, and refers to a pathway that inhibits, reduces, or eliminates immune function. Also provided are uses of the compositions for treatment, compositions for use in the treatment of a subject, including any of the compositions described herein for any of the treatments described herein.

“Solid Tumors” as described herein, has its ordinary meaning as understood in light of the specification, and refers to a malignant cancerous mass of tissue. In some embodiments of the methods of treating, ameliorating, or inhibiting a non-B cell related disease in a subject provided herein, the method including introducing, providing, or administering any one or more of the cells or compositions of any of the embodiments herein or the cells made by any one or more of the methods of the embodiments herein into a subject for therapy. In some embodiments, the subject has a cancer. In some embodiments, the cancer is a solid tumor. In some embodiments, the solid tumor is selected from the group consisting of a breast cancer, brain cancer, lung cancer, liver cancer, stomach cancer, spleen cancer, colon cancer, renal cancer, pancreatic cancer, prostate cancer, uterine cancer, skin cancer, head cancer, neck cancer, sarcomas, neuroblastomas, and ovarian cancer.

“Engraftment” as described herein, has its ordinary meaning as understood in light of the specification, and refers to the incorporation of grafted tissue into the body of the host. Several characteristics of effective CAR T-cells include showing signs of adequate engraftment, which is required for responses. For example, detection of the CAR transgene by polymerase chain reaction is not informative about the surface expression of the CAR, which is the only form that matters for efficacy. Thus, the availability of reagents to specifically detect CARs at the cell surface by flow cytometry or other methods known to those skilled in the art is crucial to understand the activity and engraftment of CAR T-cells. In the embodiments described herein, the therapeutic potency of the adoptively transferred CARs are improved by allowing a B-cell targeting CAR to drive the activation, proliferation and dispersion of infused CAR T-cells that have a second CAR that provides for redirected killing of the solid tumor. In some embodiments described herein, the methods and cells including a CAR with B-cell specificity led to the surprising effect of having an improved level of engraftment compared to T-cells that only included CARs specific for a tumor ligand. As described in the embodiments herein, the obstacle of failure to exhibit engraftment is overcome by allowing a B cell targeting CAR to drive the activation, proliferation and dispersion of infused CAR T-cells that have a CAR that provides for redirected killing of the solid tumor.

“Subject” or “patient,” as described herein, has its ordinary meaning as understood in light of the specification, and refers to any organism upon which the embodiments described herein may be used or administered, e.g., for experimental, diagnostic, prophylactic, and/or therapeutic purposes. Subjects or patients include, for example, animals. In some embodiments, the subject is mice, rats, rabbits, non-human primates, and humans. In some embodiments, the subject is a cow, sheep, pig, horse, dog, cat, primate, or a human.

As used herein, the terms “treat,” “treating,” “treated,” or “treatment” has its ordinary meaning as understood in light of the specification, and refer to both therapeutic treatment and prophylactic or preventative treatment.

As used herein, the terms “ameliorate,” “ameliorating,” “amelioration,” or “ameliorated” has its ordinary meaning as understood in light of the specification, and in reference to cancer can mean reducing the symptoms of the cancer, reducing the size of a tumor, completely or partially removing the tumor (e.g., a complete or partial response), causing stable disease, preventing progression of the cancer (e.g., progression free survival), or any other effect on the cancer that would be considered by a physician to be a therapeutic, prophylactic, or preventative treatment of the cancer.

As used herein, the terms “administer,” administering,” or “administered” has its ordinary meaning as understood in light of the specification, and includes all means of introducing the compound, or pharmaceutically acceptable salt thereof, or CAR T cell composition, wherein the CAR T cell composition includes CAR T cells and wherein the CAR includes an E2 anti-fluorescein antibody fragment, to the patient, including, but not limited to, oral, intravenous, intramuscular, subcutaneous, and transdermal.

As used herein, the terms “transduction” and “transfection” has its ordinary meaning as understood in light of the specification, and are used equivalently and the terms mean introducing a nucleic acid into a cell by any artificial method, including viral and non-viral methods.

An “effective amount” of an agent, e.g., a pharmaceutical formulation, agent, cells, or composition, in the context of administration, refers to an amount effective, at dosages/amounts and for periods of time necessary, to achieve a desired result, such as a therapeutic or prophylactic result.

A “therapeutically effective amount” of a composition, e.g., a pharmaceutical formulation including agents or cells, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the agents or populations of cells administered. In some embodiments, the provided methods involve administering the agents, cells and/or compositions at effective amounts, e.g., therapeutically effective amounts.

As used herein, the term “standard of care”, “best practice” and “standard therapy” refers to the treatment that is accepted by medical practitioners to be an appropriate, proper, effective, and/or widely used treatment for a certain disease. The standard of care of a certain disease depends on many different factors, including the biological effect of treatment, region or location within the body, patient status (e.g. age, weight, gender, hereditary risks, other disabilities, secondary conditions), toxicity, metabolism, bioaccumulation, therapeutic index, dosage, and other factors known in the art. Determining a standard of care for a disease is also dependent on establishing safety and efficacy in clinical trials as standardized by regulatory bodies such as the US Food and Drug Administration, International Council for Harmonisation, Health Canada, European Medicines Agency, Therapeutics Goods Administration, Central Drugs Standard Control Organization, National Medical Products Administration, Pharmaceuticals and Medical Devices Agency, Ministry of Food and Drug Safety, and the World Health Organization. The standard of care for a disease may include but is not limited to surgery, radiation, chemotherapy, targeted therapy, or immunotherapy (e.g., PD1/PDL1 or CTLA4 blockade therapy).

The above description discloses several methods and materials of the embodiments described herein. Some embodiments provided herein are susceptible to modifications in the methods and materials, as well as alterations in the fabrication methods and equipment. Such modifications will become apparent to those skilled in the art from a consideration of this disclosure or practice of the embodiments disclosed herein. Consequently, it is not intended that this disclosure be limited to the specific embodiments disclosed herein, but that it covers all modifications and alternatives coming within the true scope and spirit of the disclosure.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

In another embodiment of the methods described herein, any of the methods described herein can be used alone, or any of the methods described herein can be used in combination with any other method or methods described herein.

Some embodiments provided herein are further described in the following enumerated alternatives.
1. A protein construct capable of dimerizing, comprising: a receptor-specific targeting region; a transmembrane domain; an intracellular signaling domain; and an Fv domain comprising at least one dimerization interface, wherein the at least one dimerization interface is capable of forming at least one disulfide bond, and wherein the protein forms a dimer upon expression ex vivo, in vivo, or in vitro.
2. The protein of alternative 1, wherein the dimer is a heterodimer.
3. The protein construct of alternative 2, wherein the heterodimer is stabilized by at least 1, 2, 3, 4, 5, or 10 disulfide bonds.
4. The protein construct of any one of alternatives 1-3, wherein the protein construct comprises a sequence with at least 80% identity to any one of SEQ ID NOs: 1-12.
5. The protein construct of alternative 4, wherein the protein construct further comprises a VH domain and a VL domain with at least 80% identity to any one of SEQ ID NOs: 83-96.
6. The protein construct of any one of alternatives 1-5, wherein dimerization stabilizes the heavy and the light chain regions of the Fv domain.
7. The protein construct of any one of alternatives 1-6, wherein the protein is expressed in a cell, tissue, and/or organ.
8. The protein construct of any one of alternatives 1-7, wherein the protein is expressed in a mammal and/or human.
9. The protein construct of any one of alternatives 1-8, wherein the receptor-specific targeting region is functionalized only upon dimerization of the protein.
10. The protein construct of any one of alternatives 1-9, wherein the receptor-specific targeting region targets at least one of a CTLA-4 receptor, a TIM-3 receptor, a TIGIT receptor, a LAG-3 receptor, a BTLA receptor, a 2B4 receptor, a CD160 receptor, a CD226 receptor, a KIR2DL4 receptor, a CD244 receptor, a TGF-β receptor, and/or a PD1 receptor.
11. The protein construct of any one of alternatives 9-10, wherein binding of the receptor-specific targeting region blocks at least one immunoinhibitory pathway.
12. The protein construct of any one of alternatives 1-11, wherein the intracellular signaling domain is functionalized upon dimerization of the protein.
13. The protein construct of alternative 12, wherein activation of the intracellular signaling domain promotes cytokine signaling.
14. The protein construct of alternative 13, wherein at least 1, 2, 3, 4, 5, or 10 cytokines are upregulated upon activation of the intracellular signaling domain.
15. The protein construct of any one of alternatives 1-14, wherein the protein construct is a chimeric antigen receptor (CAR).
16. The protein construct of any one of alternatives 1-15, wherein the protein comprises an amino acid sequence having at least 90% sequence identity to any one of SEQ ID NOs: 1, 5, 17, and/or 18.
17. The protein construct of any one of alternatives 1-16, wherein at least one disulfide bond is formed between pair(s) of cysteine residues selected from the group consisting of: VH37 and VL95; VH44 and VL100; VH44 and VL105; VH45 and VL87; VH55 and VL101; VH100 and VL50; VH100b and VL49; VH98 and VL46; and/or VH105 and VL43; wherein the residue numbering is as determined using the Kabat numbering system.
18. A nucleotide sequence encoding the protein construct of any one of alternatives 1-17.
19. A vector encoding the nucleotide sequence of alternative 18.
20. A cell comprising any one of the protein constructs of alternatives 1-19, the nucleotide sequence of alternative 18, and/or the vector of alternative 19.
21. The cell of alternative 20, wherein the cell is a T lymphocyte or a cytotoxic T lymphocyte.
22. A method of treating cancer in a patient, the method comprising administering to the patient a cell of any one of alternatives 20-21.
23. A method of treating a disease or disorder in a patient, the method comprising: administering to the patient a cell expressing a protein construct, the protein construct comprising: a receptor-specific targeting region; a transmembrane domain; an intracellular signaling domain; and an Fv domain comprising at least one dimerization interface, wherein the at least one dimerization interface is capable of forming at least one disulfide bond, and wherein the protein construct is functional as part of a heterodimer.
24. The method of alternative 23, wherein the heterodimer is stabilized by at least 1, 2, 3, 4, 5, or 10 disulfide bonds.
25. The method of any one of alternatives 23-24, wherein dimerization stabilizes the heavy and the light chain regions of the Fv domain and functionalizes the protein construct.
26. The method of any one of alternatives 23-25, wherein the receptor-specific targeting region targets at least one of a CTLA-4 receptor, a TIM-3 receptor, a TIGIT receptor, a LAG-3 receptor, a BTLA receptor, a 2B4 receptor, a CD160 receptor, a CD226 receptor, a KIR2DL4 receptor, a CD244 receptor, a TGF-β receptor, and/or a PD1 receptor.
27. The method of any one of alternatives 23-26, wherein binding of the receptor-specific targeting region blocks at least one immunoinhibitory pathway.
28. The method of any one of alternatives 23-27, wherein activation of the intracellular signaling domain promotes cytokine signaling.
29. The method of any one of alternatives 23-28, wherein the disease or disorder is a cancer.
30. The method of alternative 29, wherein the cancer is glioblastoma, mesothelioma, pancreatic cancer, gastric cancer, blood cancer, lymphoma, multiple myeloma, leukemia diffuse large B-cell lymphoma (DLBCL), follicular lymphoma, mantle cell lymphoma, multiple myeloma, B-cell acute lymphoblastic leukemia (ALL), Large B-cell lymphoma transformed from follicular lymphoma, High grade B-cell lymphoma, Aggressive B-cell lymphoma not otherwise specified (NOS), lung cancer, ovarian cancer, breast cancer, prostate cancer, liver cancer, kidney cancer, stomach cancer, or colon cancer.

### EXAMPLES

While the present disclosure has been described in some detail for purposes of clarity and understanding, one skilled in the art will appreciate that various changes in form and detail can be made without departing from the true scope of the embodiments described herein.

### Example 1: Generation of Protein Constructs

A series of modified antibodies and FAB antibody fragments were generated. The antibodies were labeled “CM015-CM026”, and their sequences are as shown in the below Tables 2-3.

TABLE 2
Amino acid sequences of CM015-CM026 constructs (SEQ ID NO: 1-12)

| | SEQ ID | |
|---|---|---|
| Construct: | NO: | Sequence: |
| CM015 | 1 | MGTSLLCWMALCLLGADHADAYPYDVPDYASGGGGSPTTT |
| | | PAPRPPTPAPTIASQPLSLRPEACRPAAGGAVHTRGLDFA |
| | | CDIYIWAPLAGTCGVLLLSLVITLYCNHRNRRRVCKCPRP |
| | | VVEGRGSLLTCGDVEENPGPMGWSCIILFLVATATGVHSE |
| | | IVLTQSPATLSLSPGERATLSCRASQSVRSYLAWYQQKPG |
| | | QAPRLLIYDASNRATGIPARFSGSGSGTDFTLTISSLEPE |
| | | DFAVYYCQQRSNWPPTFGCGTKVEIKEPKSSDKTHTSPPS |
| | | PAPELNTSKENPFLFALEAVVISVGSMGLIISLLCVYFWL |
| | | ERTMPRIPTLKNLEDLVTEYHGNFSAWSGVSKGLAESLQP |
| | | DYSERLCLVSEIPPKGGALGEGPGASPCNQHSPYWAPPCY |
| | | TLKPETATNFSLLKQAGDVEENPGPMETDTLILWVLLLLV |
| | | PGSTGQLQVQESGPGLVKPSETLSLTCTVSGGSISNSYFS |
| | | WGWIRQPPGKCLEWIGSFYYGEKTYYNPSLKSRATISIDT |
| | | SKSQFSLKLSSVTAADTAVYYCPRGPTMIRGVIDSWGQGT |
| | | LVTVSSEPKSSDKTHTSPPSPAPELNNSSGEMDPILLTIS |
| | | ILSFFSVALLVILACVLWKKRIKPIVWPSLPDHKKTLEHL |
| | | CKKPRKNLNVSFNPESFLDCQIHRVDDIQARDEVEGFLQD |
| | | TFPQQLEESEKQRLGGDVQSPNCPSEDVVITPESFGRDSS |
| | | LTCLAGNVSACDAPILSSSRSLDCRESGKNGPHVYQDLLL |
| | | SLGTTNSTLPPPFSLQSGILTLNPVAQGQPILTSLGSNQE |
| | | EAYVTMSSFYQNQ |
| CM016 | 2 | MGTSLLCWMALCLLGADHADAYPYDVPDYASGGGGSPTTT |
| | | PAPRPPTPAPTIASQPLSLRPEACRPAAGGAVHTRGLDFA |
| | | CDIYIWAPLAGTCGVLLLSLVITLYCNHRNRRRVCKCPRP |
| | | VVEGRGSLLTCGDVEENPGPMGWSCIILFLVATATGVHSE |
| | | IVLTQSPATLSLSPGERATLSCRASQSVRSYLAWYQQKPG |
| | | QAPRLLIYDASNRATGIPARFSGSGSGTDFTLTISSLEPE |
| | | DFAVYYCQQRSNWPPTFGCGTKVEIKEPKSSDKTHTSPPS |
| | | PAPELNNSSGEMDPILLTISILSFFSVALLVILACVLWKK |
| | | RIKPIVWPSLPDHKKTLEHLCKKPRKNLNVSFNPESFLDC |
| | | QIHRVDDIQARDEVEGFLQDTFPQQLEESEKQRLGGDVQS |
| | | PNCPSEDVVITPESFGRDSSLTCLAGNVSACDAPILSSSR |
| | | SLDCRESGKNGPHVYQDLLLSLGTTNSTLPPPFSLQSGIL |
| | | TLNPVAQGQPILTSLGSNQEEAYVTMSSFYQNQATNFSLL |
| | | KQAGDVEENPGPMETDTLILWVLLLLVPGSTGQLQVQESG |
| | | PGLVKPSETLSLTCTVSGGSISNSYFSWGWIRQPPGKCLE |
| | | WIGSFYYGEKTYYNPSLKSRATISIDTSKSQFSLKLSSVT |
| | | AADTAVYYCPRGPTMIRGVIDSWGQGTLVTVSSEPKSSDK |
| | | THTSPPSPAPELNTSKENPFLFALEAVVISVGSMGLIISL |
| | | LCVYFWLERTMPRIPTLKNLEDLVTEYHGNFSAWSGVSKG |
| | | LAESLQPDYSERLCLVSEIPPKGGALGEGPGASPCNQHSP |
| | | YWAPPCYTLKPET |
| CM017 | 3 | MGTSLLCWMALCLLGADHADAYPYDVPDYASGGGGSPTTT |
| | | PAPRPPTPAPTIASQPLSLRPEACRPAAGGAVHTRGLDFA |
| | | CDIYIWAPLAGTCGVLLLSLVITLYCNHRNRRRVCKCPRP |
| | | VVEGRGSLLTCGDVEENPGPMGWSCIILFLVATATGVHSE |
| | | IVLTQSPATLSLSPGERATLSCRASQSVRSYLAWYQQKPG |
| | | QAPRLLIYDASNRATGIPARFSGSGSGTDFTLTISSLEPE |
| | | DFAVYYCQQRSNWPPTFGCGTKVEIKEPKSSDKTHTSPPS |
| | | PAPELNTSKENPFLFALEAVVISVGSMGLIISLLCVYFWL |
| | | ERTMPREKVKTLKAQNSELASTANMLREQVAQLIPTLKNL |
| | | EDLVTEYHGNFSAWSGVSKGLAESLQPDYSERLCLVSEIP |
| | | PKGGALGEGPGASPCNQHSPYWAPPCYTLKPETATNFSLL |
| | | KQAGDVEENPGPMETDTLILWVLLLLVPGSTGQLQVQESG |
| | | PGLVKPSETLSLTCTVSGGSISNSYFSWGWIRQPPGKCLE |
| | | WIGSFYYGEKTYYNPSLKSRATISIDTSKSQFSLKLSSVT |
| | | AADTAVYYCPRGPTMIRGVIDSWGQGTLVTVSSEPKSSDK |
| | | THTSPPSPAPELNNSSGEMDPILLTISILSFFSVALLVIL |
| | | ACVLWKKRIKPAETDQLEDEKSALQTEIANLLKEKEKLIV |
| | | WPSLPDHKKTLEHLCKKPRKNLNVSFNPESFLDCQIHRVD |
| | | DIQARDEVEGFLQDTFPQQLEESEKQRLGGDVQSPNCPSE |
| | | DVVITPESFGRDSSLTCLAGNVSACDAPILSSSRSLDCRE |
| | | SGKNGPHVYQDLLLSLGTTNSTLPPPFSLQSGILTLNPVA |
| | | QGQPILTSLGSNQEEAYVTMSSFYQNQ |
| CM018 | 4 | MGTSLLCWMALCLLGADHADAYPYDVPDYASGGGGSPTTT |
| | | PAPRPPTPAPTIASQPLSLRPEACRPAAGGAVHTRGLDFA |
| | | CDIYIWAPLAGTCGVLLLSLVITLYCNHRNRRRVCKCPRP |
| | | VVEGRGSLLTCGDVEENPGPMGWSCIILFLVATATGVHSE |
| | | IVLTQSPATLSLSPGERATLSCRASQSVRSYLAWYQQKPG |
| | | QAPRLLIYDASNRATGIPARFSGSGSGTDFTLTISSLEPE |
| | | DFAVYYCQQRSNWPPTFGCGTKVEIKEPKSSDKTHTSPPS |
| | | PAPELNNSSGEMDPILLTISILSFFSVALLVILACVLWKK |
| | | RIKPAETDQLEDEKSALQTEIANLLKEKEKLIVWPSLPDH |
| | | KKTLEHLCKKPRKNLNVSFNPESFLDCQIHRVDDIQARDE |
| | | VEGFLQDTFPQQLEESEKQRLGGDVQSPNCPSEDVVITPE |
| | | SFGRDSSLTCLAGNVSACDAPILSSSRSLDCRESGKNGPH |
| | | VYQDLLLSLGTTNSTLPPPFSLQSGILTLNPVAQGQPILT |
| | | SLGSNQEEAYVTMSSFYQNQATNFSLLKQAGDVEENPGPM |
| | | ETDTLILWVLLLLVPGSTGQLQVQESGPGLVKPSETLSLT |
| | | CTVSGGSISNSYFSWGWIRQPPGKCLEWIGSFYYGEKTYY |
| | | NPSLKSRATISIDTSKSQFSLKLSSVTAADTAVYYCPRGP |
| | | TMIRGVIDSWGQGTLVTVSSEPKSSDKTHTSPPSPAPELN |
| | | TSKENPFLFALEAVVISVGSMGLIISLLCVYFWLERTMPR |
| | | EKVKTLKAQNSELASTANMLREQVAQLIPTLKNLEDLVTE |
| | | YHGNFSAWSGVSKGLAESLQPDYSERLCLVSEIPPKGGAL |
| | | GEGPGASPCNQHSPYWAPPCYTLKPET |
| CM019 | 5 | MGTSLLCWMALCLLGADHADAYPYDVPDYASGGGGSPTTT |
| | | PAPRPPTPAPTIASQPLSLRPEACRPAAGGAVHTRGLDFA |
| | | CDIYIWAPLAGTCGVLLLSLVITLYCNHRNRRRVCKCPRP |
| | | VVEGRGSLLTCGDVEENPGPMGWSCIILFLVATATGVHSE |
| | | IVLTQSPATLSLSPGERATLSCRASQSVRSYLAWYQQKPG |
| | | QAPRLLIYDASNRATGIPARFSGSGSGTDFTLTISSLEPE |
| | | DFAVYYCQQRSNWPPTFGCGTKVEIKEPKSSDKTHTSPPS |
| | | PAPELNGIWSEWSEARSWDTESVLPMWVLALIVIFLTIAV |
| | | LLALRFCGIYGYRLRRKWEEKIPNPSKSHLFQNGSAELWP |
| | | PGSMSAFTSGSPPHQGPWGSRFPELEGVFPVGFGDSEVSP |
| | | LTIEDPKHVCDPPSGPDTTPAASDLPTEQPPSPQPGPPAA |
| | | SHTPEKQASSFDFNGPYLGPPHSRSLPDILGQPEPPQEGG |
| | | SQKSPPPGSLEYLCLPAGGQVQLVPLAQAMGPGQAVEVER |
| | | RPSQGAAGSPSLESGGGPAPPALGPRVGGQDQKDSPVAIP |
| | | MSSGDTEDPGVASGYVSSADLVFTPNSGASSVSLVPSLGL |
| | | PSDQTPSLCPGLASGPPGAPGPVKSGFEGYVELPPIEGRS |
| | | PRSPRNNPVPPEAKSPVLNPGERPADVSPTSPQPEGLLVL |
| | | QQVGDYCFLPGLGPGPLSLRSKPSSPGPGPEIKNLDQAFQ |
| | | VKKPPGQAVPQVPVIQLFKALKQQDYLSLPPWEVNKPGEV |
| | | CATNFSLLKQAGDVEENPGPMETDTLILWVLLLLVPGSTG |
| | | QLQVQESGPGLVKPSETLSLTCTVSGGSISNSYFSWGWIR |
| | | QPPGKCLEWIGSFYYGEKTYYNPSLKSRATISIDTSKSQF |
| | | SLKLSSVTAADTAVYYCPRGPTMIRGVIDSWGQGTLVTVS |
| | | SEPKSSDKTHTSPPSPAPELNWSSWSEAIEFGSDDGNLGS |
| | | VYIYVLLIVGTLVCGIVLGFLFKRFLRIQRLFPPVPQIKD |
| | | KLNDNHEVEDEIIWEEFTPEEGKGYREEVLTVKEIT |
| CM020 | 6 | MGTSLLCWMALCLLGADHADAYPYDVPDYASGGGGSPTTT |
| | | PAPRPPTPAPTIASQPLSLRPEACRPAAGGAVHTRGLDFA |
| | | CDIYIWAPLAGTCGVLLLSLVITLYCNHRNRRRVCKCPRP |
| | | VVEGRGSLLTCGDVEENPGPMGWSCIILFLVATATGVHSE |
| | | IVLTQSPATLSLSPGERATLSCRASQSVRSYLAWYQQKPG |
| | | QAPRLLIYDASNRATGIPARFSGSGSGTDFTLTISSLEPE |
| | | DFAVYYCQQRSNWPPTFGCGTKVEIKEPKSSDKTHTSPPS |
| | | PAPELNWSSWSEAIEFGSDDGNLGSVYIYVLLIVGTLVCG |
| | | IVLGFLFKRFLRIQRLFPPVPQIKDKLNDNHEVEDEIIWE |
| | | EFTPEEGKGYREEVLTVKEITATNFSLLKQAGDVEENPGP |
| | | METDTLILWVLLLLVPGSTGQLQVQESGPGLVKPSETLSL |
| | | TCTVSGGSISNSYFSWGWIRQPPGKCLEWIGSFYYGEKTY |
| | | YNPSLKSRATISIDTSKSQFSLKLSSVTAADTAVYYCPRG |
| | | PTMIRGVIDSWGQGTLVTVSSEPKSSDKTHTSPPSPAPEL |
| | | NGIWSEWSEARSWDTESVLPMWVLALIVIFLTIAVLLALR |
| | | FCGIYGYRLRRKWEEKIPNPSKSHLFQNGSAELWPPGSMS |
| | | AFTSGSPPHQGPWGSRFPELEGVFPVGFGDSEVSPLTIED |
| | | PKHVCDPPSGPDTTPAASDLPTEQPPSPQPGPPAASHTPE |
| | | KQASSFDFNGPYLGPPHSRSLPDILGQPEPPQEGGSQKSP |
| | | PPGSLEYLCLPAGGQVQLVPLAQAMGPGQAVEVERRPSQG |
| | | AAGSPSLESGGGPAPPALGPRVGGQDQKDSPVAIPMSSGD |
| | | TEDPGVASGYVSSADLVFTPNSGASSVSLVPSLGLPSDQT |
| | | PSLCPGLASGPPGAPGPVKSGFEGYVELPPIEGRSPRSPR |
| | | NNPVPPEAKSPVLNPGERPADVSPTSPQPEGLLVLQQVGD |
| | | YCFLPGLGPGPLSLRSKPSSPGPGPEIKNLDQAFQVKKPP |
| | | GQAVPQVPVIQLFKALKQQDYLSLPPWEVNKPGEVC |
| CM021 | 7 | MGTSLLCWMALCLLGADHADAYPYDVPDYASGGGGSPTTT |
| | | PAPRPPTPAPTIASQPLSLRPEACRPAAGGAVHTRGLDFA |
| | | CDIYIWAPLAGTCGVLLLSLVITLYCNHRNRRRVCKCPRP |
| | | VVEGRGSLLTCGDVEENPGPMGWSCIILFLVATATGVHSE |
| | | IVLTQSPATLSLSPGERATLSCRASQSVRSYLAWYQQKPG |
| | | QAPRLLIYDASNRATGIPARFSGSGSGTDFTLTISSLEPE |
| | | DFAVYYCQQRSNWPPTFGCGTKVEIKEPKSSDKTHTSPPS |
| | | PAPELNGIWSEWSEARSWDTESVLPMWVLALIVIFLTIAV |
| | | LLALRFCGIYEKVKTLKAQNSELASTANMLREQVAQLGYR |
| | | LRRKWEEKIPNPSKSHLFQNGSAELWPPGSMSAFTSGSPP |
| | | HQGPWGSRFPELEGVFPVGFGDSEVSPLTIEDPKHVCDPP |
| | | SGPDTTPAASDLPTEQPPSPQPGPPAASHTPEKQASSFDF |
| | | NGPYLGPPHSRSLPDILGQPEPPQEGGSQKSPPPGSLEYL |
| | | CLPAGGQVQLVPLAQAMGPGQAVEVERRPSQGAAGSPSLE |
| | | SGGGPAPPALGPRVGGQDQKDSPVAIPMSSGDTEDPGVAS |
| | | GYVSSADLVFTPNSGASSVSLVPSLGLPSDQTPSLCPGLA |
| | | SGPPGAPGPVKSGFEGYVELPPIEGRSPRSPRNNPVPPEA |
| | | KSPVLNPGERPADVSPTSPQPEGLLVLQQVGDYCFLPGLG |
| | | PGPLSLRSKPSSPGPGPEIKNLDQAFQVKKPPGQAVPQVP |
| | | VIQLFKALKQQDYLSLPPWEVNKPGEVCATNFSLLKQAGD |
| | | VEENPGPMETDTLILWVLLLLVPGSTGQLQVQESGPGLVK |
| | | PSETLSLTCTVSGGSISNSYFSWGWIRQPPGKCLEWIGSF |
| | | YYGEKTYYNPSLKSRATISIDTSKSQFSLKLSSVTAADTA |
| | | VYYCPRGPTMIRGVIDSWGQGTLVTVSSEPKSSDKTHTSP |
| | | PSPAPELNWSSWSEAIEFGSDDGNLGSVYIYVLLIVGTLV |
| | | CGIVLGFLFKRFLRAETDQLEDEKSALQTEIANLLKEKEK |
| | | LIQRLFPPVPQIKDKLNDNHEVEDEIIWEEFTPEEGKGYR |
| | | EEVLTVKEIT |
| CM022 | 8 | MGTSLLCWMALCLLGADHADAYPYDVPDYASGGGGSPTTT |
| | | PAPRPPTPAPTIASQPLSLRPEACRPAAGGAVHTRGLDFA |
| | | CDIYIWAPLAGTCGVLLLSLVITLYCNHRNRRRVCKCPRP |
| | | VVEGRGSLLTCGDVEENPGPMGWSCIILFLVATATGVHSE |
| | | IVLTQSPATLSLSPGERATLSCRASQSVRSYLAWYQQKPG |
| | | QAPRLLIYDASNRATGIPARFSGSGSGTDFTLTISSLEPE |
| | | DFAVYYCQQRSNWPPTFGCGTKVEIKEPKSSDKTHTSPPS |
| | | PAPELNWSSWSEAIEFGSDDGNLGSVYIYVLLIVGTLVCG |
| | | IVLGFLFKRFLRAETDQLEDEKSALQTEIANLLKEKEKLI |
| | | QRLFPPVPQIKDKLNDNHEVEDEIIWEEFTPEEGKGYREE |
| | | VLTVKEITATNFSLLKQAGDVEENPGPMETDTLILWVLLL |
| | | LVPGSTGQLQVQESGPGLVKPSETLSLTCTVSGGSISNSY |
| | | FSWGWIRQPPGKCLEWIGSFYYGEKTYYNPSLKSRATISI |
| | | DTSKSQFSLKLSSVTAADTAVYYCPRGPTMIRGVIDSWGQ |
| | | GTLVTVSSEPKSSDKTHTSPPSPAPELNGIWSEWSEARSW |
| | | DTESVLPMWVLALIVIFLTIAVLLALRFCGIYEKVKTLKA |
| | | QNSELASTANMLREQVAQLGYRLRRKWEEKIPNPSKSHLF |
| | | QNGSAELWPPGSMSAFTSGSPPHQGPWGSRFPELEGVFPV |
| | | GFGDSEVSPLTIEDPKHVCDPPSGPDTTPAASDLPTEQPP |
| | | SPQPGPPAASHTPEKQASSFDFNGPYLGPPHSRSLPDILG |
| | | QPEPPQEGGSQKSPPPGSLEYLCLPAGGQVQLVPLAQAMG |
| | | PGQAVEVERRPSQGAAGSPSLESGGGPAPPALGPRVGGQD |
| | | QKDSPVAIPMSSGDTEDPGVASGYVSSADLVFTPNSGASS |
| | | VSLVPSLGLPSDQTPSLCPGLASGPPGAPGPVKSGFEGYV |
| | | ELPPIEGRSPRSPRNNPVPPEAKSPVLNPGERPADVSPTS |
| | | PQPEGLLVLQQVGDYCFLPGLGPGPLSLRSKPSSPGPGPE |
| | | IKNLDQAFQVKKPPGQAVPQVPVIQLFKALKQQDYLSLPP |
| | | WEVNKPGEVC |
| CM023 | 9 | MGTSLLCWMALCLLGADHADAYPYDVPDYASGGGGSPTTT |
| | | PAPRPPTPAPTIASQPLSLRPEACRPAAGGAVHTRGLDFA |
| | | CDIYIWAPLAGTCGVLLLSLVITLYCNHRNRRRVCKCPRP |
| | | VVEGRGSLLTCGDVEENPGPMGWSCIILFLVATATGVHSE |
| | | IVLTQSPATLSLSPGERATLSCRASQSVRSYLAWYQQKPG |
| | | QAPRLLIYDASNRATGIPARFSGSGSGTDFTLTISSLEPE |
| | | DFAVYYCQQRSNWPPTFGCGTKVEIKEPKSSDKTHTSPPS |
| | | PAPELSPLFPGPSKPFWVLVVVGGVLACYSLLVTVAFIIF |
| | | WVRSATNFSLLKQAGDVEENPGPMETDTLILWVLLLLVPG |
| | | STGQLQVQESGPGLVKPSETLSLTCTVSGGSISNSYFSWG |
| | | WIRQPPGKCLEWIGSFYYGEKTYYNPSLKSRATISIDTSK |
| | | SQFSLKLSSVTAADTAVYYCPRGPTMIRGVIDSWGQGTLV |
| | | TVSSEPKSSDKTHTSPPSPAPELSPLFPGPSKPFWVLVVV |
| | | GGVLACYSLLVTVAFIIFWVRS |
| CM024 | 10 | MGTSLLCWMALCLLGADHADAYPYDVPDYASGGGGSPTTT |
| | | PAPRPPTPAPTIASQPLSLRPEACRPAAGGAVHTRGLDFA |
| | | CDIYIWAPLAGTCGVLLLSLVITLYCNHRNRRRVCKCPRP |
| | | VVEGRGSLLTCGDVEENPGPMGWSCIILFLVATATGVHSE |
| | | IVLTQSPATLSLSPGERATLSCRASQSVRSYLAWYQQKPG |
| | | QAPRLLIYDASNRATGIPARFSGSGSGTDFTLTISSLEPE |
| | | DFAVYYCQQRSNWPPTFGCGTKVEIKEPKSSDKTHTSPPS |
| | | PAPELNNSSGEMDPILLTISILSFFSVALLVILACVLWKK |
| | | ATNFSLLKQAGDVEENPGPMETDTLILWVLLLLVPGSTGQ |
| | | LQVQESGPGLVKPSETLSLTCTVSGGSISNSYFSWGWIRQ |
| | | PPGKCLEWIGSFYYGEKTYYNPSLKSRATISIDTSKSQFS |
| | | LKLSSVTAADTAVYYCPRGPTMIRGVIDSWGQGTLVTVSS |
| | | EPKSSDKTHTSPPSPAPELNTSKENPFLFALEAVVISVGS |
| | | MGLIISLLCVYFWLER |
| CM025 | 11 | MGTSLLCWMALCLLGADHADAYPYDVPDYASGGGGSPTTT |
| | | PAPRPPTPAPTIASQPLSLRPEACRPAAGGAVHTRGLDFA |
| | | CDIYIWAPLAGTCGVLLLSLVITLYCNHRNRRRVCKCPRP |
| | | VVEGRGSLLTCGDVEENPGPMGWSCIILFLVATATGVHSR |
| | | TVAAPSVFIFPPSDEQLKSGTASVVCLLNNFYPREAKVQW |
| | | KVDNALQSGNSQESVTEQDSKDSTYSLSSTLTLSKADYEK |
| | | HKVYACEVTHQGLSSPVTKSFNRGECEPKSCDKTHTCPPC |
| | | PEPKSSDKTHTSPPSPAPELNGIWSEWSEARSWDTESVLP |
| | | MWVLALIVIFLTIAVLLALRFCGIYGYRLRRKWEEKIPNP |
| | | SKSHLFQNGSAELWPPGSMSAFTSGSPPHQGPWGSRFPEL |
| | | EGVFPVGFGDSEVSPLTIEDPKHVCDPPSGPDTTPAASDL |
| | | PTEQPPSPQPGPPAASHTPEKQASSFDFNGPYLGPPHSRS |
| | | LPDILGQPEPPQEGGSQKSPPPGSLEYLCLPAGGQVQLVP |
| | | LAQAMGPGQAVEVERRPSQGAAGSPSLESGGGPAPPALGP |
| | | RVGGQDQKDSPVAIPMSSGDTEDPGVASGYVSSADLVFTP |
| | | NSGASSVSLVPSLGLPSDQTPSLCPGLASGPPGAPGPVKS |
| | | GFEGYVELPPIEGRSPRSPRNNPVPPEAKSPVLNPGERPA |
| | | DVSPTSPQPEGLLVLQQVGDYCFLPGLGPGPLSLRSKPSS |
| | | PGPGPEIKNLDQAFQVKKPPGQAVPQVPVIQLFKALKQQD |
| | | YLSLPPWEVNKPGEVCATNFSLLKQAGDVEENPGPMETDT |
| | | LILWVLLLLVPGSTGASTKGPSVFPLAPSSKSTSGGTAAL |
| | | GCLVKDYFPEPVTVSWNSGALTSGVHTFPAVLQSSGLYSL |
| | | SSVVTVPSSSLGTQTYICNVNHKPSNTKVDKKVEPKSCDK |
| | | THTCPPCPEPKSSDKTHTSPPSPAPELNWSSWSEAIEFGS |
| | | DDGNLGSVYIYVLLIVGTLVCGIVLGFLFKRFLRIQRLFP |
| | | PVPQIKDKLNDNHEVEDEIIWEEFTPEEGKGYREEVLTVK |
| | | EIT |
| CM026 | 12 | MGTSLLCWMALCLLGADHADAYPYDVPDYASGGGGSPTTT |
| | | PAPRPPTPAPTIASQPLSLRPEACRPAAGGAVHTRGLDFA |
| | | CDIYIWAPLAGTCGVLLLSLVITLYCNHRNRRRVCKCPRP |
| | | VVEGRGSLLTCGDVEENPGPMGWSCIILFLVATATGVHSD |
| | | IQMTQSPSSLSASVGDRVTITCKASQNVGINVAWYQQKPG |
| | | KAPKSLISSASYRYSGVPSRFSGSGSGTDFTLTISSLQPE |
| | | DFATYFCQQYDTYPFTFGCGTKVEIKEPKSSDKTHTSPPS |
| | | PAPELNGIWSEWSEARSWDTESVLPMWVLALIVIFLTIAV |
| | | LLALRFCGIYGYRLRRKWEEKIPNPSKSHLFQNGSAELWP |
| | | PGSMSAFTSGSPPHQGPWGSRFPELEGVFPVGFGDSEVSP |
| | | LTIEDPKHVCDPPSGPDTTPAASDLPTEQPPSPQPGPPAA |
| | | SHTPEKQASSFDFNGPYLGPPHSRSLPDILGQPEPPQEGG |
| | | SQKSPPPGSLEYLCLPAGGQVQLVPLAQAMGPGQAVEVER |
| | | RPSQGAAGSPSLESGGGPAPPALGPRVGGQDQKDSPVAIP |
| | | MSSGDTEDPGVASGYVSSADLVFTPNSGASSVSLVPSLGL |
| | | PSDQTPSLCPGLASGPPGAPGPVKSGFEGYVELPPIEGRS |
| | | PRSPRNNPVPPEAKSPVLNPGERPADVSPTSPQPEGLLVL |
| | | QQVGDYCFLPGLGPGPLSLRSKPSSPGPGPEIKNLDQAFQ |
| | | VKKPPGQAVPQVPVIQLFKALKQQDYLSLPPWEVNKPGEV |
| | | CATNFSLLKQAGDVEENPGPMETDTLILWVLLLLVPGSTG |
| | | QVQLVQSGAEEVKKPGSSVKVSCKASGYTFTTYYLHWVRQ |
| | | APGQCLEWMGWIYPGNVHAQYNEKFKGRVTITADKSTSTA |
| | | YMELSSLRSEDTAVYYCARSWEGFPYWGQGTTVTVSSEPK |
| | | SSDKTHTSPPSPAPELNWSSWSEAIEFGSDDGNLGSVYIY |
| | | VLLIVGTLVCGIVLGFLFKRFLRIQRLFPPVPQIKDKLND |
| | | NHEVEDEIIWEEFTPEEGKGYREEVLTVKEIT |

TABLE 3
DNA sequences of CM015-CM026 constructs (SEQ ID NO: 13-24)

| | SEQ ID | |
|---|---|---|
| Construct: | NO: | Sequence: |
| CM015 | 13 | atgggcaccagcctgctGTGCTGGATGGCCCTGTGCCTGCTGGGCGCCGACCACG |
| | | CCGATGCCTACCCCTACGACGTGCCCGACTACGCCAGCGGAGGGGGAGGCAGCCC |
| | | CACCACCACCCCAGCCCCCAGACCTCCCACCCCAGCCCCCACCATCGCCAGCCAG |
| | | CCTCTGAGCCTGAGACCCGAGGCCTGCCGCCCAGCgGCCGGCGGCGCCGTGCACA |
| | | CCAGAGGCCTGGATTTCGCCTGCGATATCTACATCTGGGCCCCACTcGCCGGCAC |
| | | CTGTGGCGTGCTGCTGCTGAGCCTGGTGATCACCCTGTACTGCAACCACCGCAAC |
| | | CGCAGGCGCGTGTGCAAGTGCCCCAGGCCCGTcGTGGAAGGCAGAGGCAGCCTGC |
| | | TTACATGTGGCGACGTGGAAGAGAACCCCGGACCTATGGGCTGGAGCTGCATCAT |
| | | CCTGTTCCTGGTGGCCACCGCCACCGGCGTGCACAGCGAGATCGTGCTGACACAG |
| | | TCTCCCGCCACACTGTCACTGTCTCCAGGCGAAAGAGCCACACTGAGCTGTAGAG |
| | | CCTCTCAGAGCGTGCGGTCTTACCTGGCCTGGTATCAGCAGAAGCCTGGACAGGC |
| | | TCCCCGGCTGCTGATCTACGATGCCAGCAATAGAGCCACAGGCATCCCCGCCAGA |
| | | TTTTCTGGCTCTGGCAGCGGCACCGATTTCACCCTGACCATAAGCAGCCTGGAAC |
| | | CTGAGGACTTCGCCGTGTACTACTGCCAGCAGAGAAGCAACTGGCCTCCTACCTT |
| | | TGGCTGCGGCACCAAGGTGGAAATCAAGGAGCCCAAGAGCAGCGACAAGACCCAC |
| | | ACCAGCCCCCCCAGCCCCGCCCCCGAGCTGAACACCAGCAAGGAAAACCCCTTTC |
| | | TGTTCGCCCTGGAGGCCGTGGTTATCAGCGTGGGCTCTATGGGCCTGATCATCAG |
| | | CCTGCTGTGCGTGTACTTCTGGCTGGAACGGACCATGCCTAGAATCCCCACACTG |
| | | AAGAACCTTGAGGACCTGGTCACCGAGTACCACGGCAACTTTTCTGCTTGGAGCG |
| | | GCGTGTCCAAAGGCCTGGCCGAATCTCTCCAGCCTGACTACAGCGAGAGACTGTG |
| | | CCTGGTGTCTGAGATCCCTCCTAAAGGCGGAGCCCTCGGAGAAGGACCTGGCGCC |
| | | TCTCCATGTAATCAGCACAGCCCTTACTGGGCTCCTCCTTGCTACACCCTGAAGC |
| | | CTGAGACAGCCACCAATTTCAGCCTGCTGAAACAGGCTGGCGACGTGGAAGAGAA |
| | | CCCTGGACCTATGGAGACCGACACCCTGATCCTGTGGGTGCTGCTGCTGCTGGTG |
| | | CCCGGCAGCACCGGCCAGCTCCAGGTGCAAGAGTCTGGACCTGGCCTGGTCAAGC |
| | | CTAGCGAGACACTGAGCCTGACCTGTACAGTGTCTGGCGGCAGCATCAGCAACAG |
| | | CTACTTTAGCTGGGGCTGGATCAGACAGCCTCCTGGCAAGTGCCTGGAATGGATC |
| | | GGCAGCTTCTACTACGGCGAGAAAACCTACTACAACCCCAGCCTGAAGTCCCGGG |
| | | CCACCATCAGCATCGATACCAGCAAGAGCCAGTTCTCCCTGAAGCTGAGCAGCGT |
| | | GACAGCCGCCGATACAGCCGTGTACTACTGTCCTAGAGGCCCCACCATGATCCGG |
| | | GGCGTGATAGATTCTTGGGGCCAGGGCACACTGGTCACCGTTTCTTCTGAGCCGA |
| | | AAtcctccGATAAAACGCATACAtccCCGCCATCACCAGCGCCAGAACTGAACAA |
| | | CAGCTCCGGCGAAATGGACCCTATCCTGCTGACAATCAGCATCCTGAGCTTTTTC |
| | | AGCGTGGCCCTGCTGGTCATCCTGGCCTGTGTGCTGTGGAAGAAGCGGATCAAGC |
| | | CCATCGTGTGGCCCAGCCTGCCTGACCACAAGAAAACCCTGGAACACCTGTGCAA |
| | | GAAGCCCCGGAAGAACCTGAACGTGTCCTTCAATCCCGAGAGCTTCCTGGACTGC |
| | | CAGATCCACAGAGTGGACGACATCCAGGCCAGGGACGAAGTGGAAGGCTTCCTCC |
| | | AGGACACATTCCCTCAGCAGCTGGAAGAGAGCGAGAAGCAGAGACTCGGCGGAGA |
| | | TGTGCAGAGCCCTAATTGCCCTAGCGAGGACGTGGTCATCACCCCTGAGAGCTTC |
| | | GGCAGAGATAGCAGCCTGACATGTCTGGCCGGCAATGTGTCCGCCTGTGATGCCC |
| | | CTATCCTGAGCAGTAGCAGAAGCCTGGATTGCAGAGAGAGCGGCAAGAACGGCCC |
| | | TCACGTGTACCAGGATCTGCTGCTGTCTCTGGGCACCACCAATAGCACACTGCCT |
| | | CCACCTTTCAGCCTGCAAAGCGGCATCCTGACACTGAACCCTGTGGCTCAGGGAC |
| | | AGCCAATCCTGACAAGCCTGGGCAGCAATCAAGAAGAGGCCTACGTCACCATGAG |
| | | CAGCTTCTACCAGAACCAG |
| CM016 | 14 | atgggcaccagcctgctGTGCTGGATGGCCCTGTGCCTGCTGGGCGCCGACCACG |
| | | CCGATGCCTACCCCTACGACGTGCCCGACTACGCCAGCGGAGGGGGAGGCAGCCC |
| | | CACCACCACCCCAGCCCCCAGACCTCCCACCCCAGCCCCCACCATCGCCAGCCAG |
| | | CCTCTGAGCCTGAGACCCGAGGCCTGCCGCCCAGCgGCCGGCGGCGCCGTGCACA |
| | | CCAGAGGCCTGGATTTCGCCTGCGATATCTACATCTGGGCCCCACTcGCCGGCAC |
| | | CTGTGGCGTGCTGCTGCTGAGCCTGGTGATCACCCTGTACTGCAACCACCGCAAC |
| | | CGCAGGCGCGTGTGCAAGTGCCCCAGGCCCGTcGTGGAAGGCAGAGGCAGCCTGC |
| | | TTACATGTGGCGACGTGGAAGAGAACCCCGGACCTATGGGCTGGAGCTGCATCAT |
| | | CCTGTTCCTGGTGGCCACCGCCACCGGCGTGCACAGCGAGATCGTGCTGACACAG |
| | | TCTCCCGCCACACTGTCACTGTCTCCAGGCGAAAGAGCCACACTGAGCTGTAGAG |
| | | CCTCTCAGAGCGTGCGGTCTTACCTGGCCTGGTATCAGCAGAAGCCTGGACAGGC |
| | | TCCCCGGCTGCTGATCTACGATGCCAGCAATAGAGCCACAGGCATCCCCGCCAGA |
| | | TTTTCTGGCTCTGGCAGCGGCACCGATTTCACCCTGACCATAAGCAGCCTGGAAC |
| | | CTGAGGACTTCGCCGTGTACTACTGCCAGCAGAGAAGCAACTGGCCTCCTACCTT |
| | | TGGCTGCGGCACCAAGGTGGAAATCAAGGAGCCCAAGAGCAGCGACAAGACCCAC |
| | | ACCAGCCCCCCCAGCCCCGCCCCCGAGCTGAACAACAGCTCCGGCGAAATGGACC |
| | | CTATCCTGCTGACAATCAGCATCCTGAGCTTTTTCAGCGTGGCCCTGCTGGTCAT |
| | | CCTGGCCTGTGTGCTGTGGAAGAAGCGGATCAAGCCCATCGTGTGGCCCAGCCTG |
| | | CCTGACCACAAGAAAACCCTGGAACACCTGTGCAAGAAGCCCCGGAAGAACCTGA |
| | | ACGTGTCCTTCAATCCCGAGAGCTTCCTGGACTGCCAGATCCACAGAGTGGACGA |
| | | CATCCAGGCCAGGGACGAAGTGGAAGGCTTCCTCCAGGACACATTCCCTCAGCAG |
| | | CTGGAAGAGAGCGAGAAGCAGAGACTCGGCGGAGATGTGCAGAGCCCTAATTGCC |
| | | CTAGCGAGGACGTGGTCATCACCCCTGAGAGCTTCGGCAGAGATAGCAGCCTGAC |
| | | ATGTCTGGCCGGCAATGTGTCCGCCTGTGATGCCCCTATCCTGAGCAGTAGCAGA |
| | | AGCCTGGATTGCAGAGAGAGCGGCAAGAACGGCCCTCACGTGTACCAGGATCTGC |
| | | TGCTGTCTCTGGGCACCACCAATAGCACACTGCCTCCACCTTTCAGCCTGCAAAG |
| | | CGGCATCCTGACACTGAACCCTGTGGCTCAGGGACAGCCAATCCTGACAAGCCTG |
| | | GGCAGCAATCAAGAAGAGGCCTACGTCACCATGAGCAGCTTCTACCAGAACCAGG |
| | | CCACCAATTTCAGCCTGCTGAAACAGGCTGGCGACGTGGAAGAGAACCCTGGACC |
| | | TATGGAGACCGACACCCTGATCCTGTGGGTGCTGCTGCTGCTGGTGCCCGGCAGC |
| | | ACCGGCCAGCTCCAGGTGCAAGAGTCTGGACCTGGCCTGGTCAAGCCTAGCGAGA |
| | | CACTGAGCCTGACCTGTACAGTGTCTGGCGGCAGCATCAGCAACAGCTACTTTAG |
| | | CTGGGGCTGGATCAGACAGCCTCCTGGCAAGTGCCTGGAATGGATCGGCAGCTTC |
| | | TACTACGGCGAGAAAACCTACTACAACCCCAGCCTGAAGTCCCGGGCCACCATCA |
| | | GCATCGATACCAGCAAGAGCCAGTTCTCCCTGAAGCTGAGCAGCGTGACAGCCGC |
| | | CGATACAGCCGTGTACTACTGTCCTAGAGGCCCCACCATGATCCGGGGCGTGATA |
| | | GATTCTTGGGGCCAGGGCACACTGGTCACCGTTTCTTCTGAGCCGAAAtcctccG |
| | | ATAAAACGCATACAtccCCGCCATCACCAGCGCCAGAACTGAACACCAGCAAGGA |
| | | AAACCCCTTTCTGTTCGCCCTGGAGGCCGTGGTTATCAGCGTGGGCTCTATGGGC |
| | | CTGATCATCAGCCTGCTGTGCGTGTACTTCTGGCTGGAACGGACCATGCCTAGAA |
| | | TCCCCACACTGAAGAACCTTGAGGACCTGGTCACCGAGTACCACGGCAACTTTTC |
| | | TGCTTGGAGCGGCGTGTCCAAAGGCCTGGCCGAATCTCTCCAGCCTGACTACAGC |
| | | GAGAGACTGTGCCTGGTGTCTGAGATCCCTCCTAAAGGCGGAGCCCTCGGAGAAG |
| | | GACCTGGCGCCTCTCCATGTAATCAGCACAGCCCTTACTGGGCTCCTCCTTGCTA |
| | | CACCCTGAAGCCTGAGACA |
| CM017 | 15 | atgggcaccagcctgctGTGCTGGATGGCCCTGTGCCTGCTGGGCGCCGACCACG |
| | | CCGATGCCTACCCCTACGACGTGCCCGACTACGCCAGCGGAGGGGGAGGCAGCCC |
| | | CACCACCACCCCAGCCCCCAGACCTCCCACCCCAGCCCCCACCATCGCCAGCCAG |
| | | CCTCTGAGCCTGAGACCCGAGGCCTGCCGCCCAGCgGCCGGCGGCGCCGTGCACA |
| | | CCAGAGGCCTGGATTTCGCCTGCGATATCTACATCTGGGCCCCACTcGCCGGCAC |
| | | CTGTGGCGTGCTGCTGCTGAGCCTGGTGATCACCCTGTACTGCAACCACCGCAAC |
| | | CGCAGGCGCGTGTGCAAGTGCCCCAGGCCCGTcGTGGAAGGCAGAGGCAGCCTGC |
| | | TTACATGTGGCGACGTGGAAGAGAACCCCGGACCTATGGGCTGGAGCTGCATCAT |
| | | CCTGTTCCTGGTGGCCACCGCCACCGGCGTGCACAGCGAGATCGTGCTGACACAG |
| | | TCTCCCGCCACACTGTCACTGTCTCCAGGCGAAAGAGCCACACTGAGCTGTAGAG |
| | | CCTCTCAGAGCGTGCGGTCTTACCTGGCCTGGTATCAGCAGAAGCCTGGACAGGC |
| | | TCCCCGGCTGCTGATCTACGATGCCAGCAATAGAGCCACAGGCATCCCCGCCAGA |
| | | TTTTCTGGCTCTGGCAGCGGCACCGATTTCACCCTGACCATAAGCAGCCTGGAAC |
| | | CTGAGGACTTCGCCGTGTACTACTGCCAGCAGAGAAGCAACTGGCCTCCTACCTT |
| | | TGGCTGCGGCACCAAGGTGGAAATCAAGGAGCCCAAGAGCAGCGACAAGACCCAC |
| | | ACCAGCCCCCCCAGCCCCGCCCCCGAGCTGAACACCAGCAAGGAAAACCCCTTTC |
| | | TGTTCGCCCTGGAGGCCGTGGTTATCAGCGTGGGCTCTATGGGCCTGATCATCAG |
| | | CCTGCTGTGCGTGTACTTCTGGCTGGAACGGACCATGCCTAGAGAGAAAGTCAAG |
| | | ACCCTGAAGGCCCAGAACAGCGAGCTGGCCTCTACCGCCAACATGCTGAGAGAAC |
| | | AGGTGGCCCAACTGATCCCCACACTGAAGAACCTTGAGGACCTGGTCACCGAGTA |
| | | CCACGGCAACTTTTCTGCTTGGAGCGGCGTGTCCAAAGGCCTGGCCGAATCTCTC |
| | | CAGCCTGACTACAGCGAGAGACTGTGCCTGGTGTCTGAGATCCCTCCTAAAGGCG |
| | | GAGCCCTCGGAGAAGGACCTGGCGCCTCTCCATGTAATCAGCACAGCCCTTACTG |
| | | GGCTCCTCCTTGCTACACCCTGAAGCCTGAGACAGCCACCAATTTCAGCCTGCTG |
| | | AAACAGGCTGGCGACGTGGAAGAGAACCCTGGACCTATGGAGACCGACACCCTGA |
| | | TCCTGTGGGTGCTGCTGCTGCTGGTGCCCGGCAGCACCGGCCAGCTCCAGGTGCA |
| | | AGAGTCTGGACCTGGCCTGGTCAAGCCTAGCGAGACACTGAGCCTGACCTGTACA |
| | | GTGTCTGGCGGCAGCATCAGCAACAGCTACTTTAGCTGGGGCTGGATCAGACAGC |
| | | CTCCTGGCAAGTGCCTGGAATGGATCGGCAGCTTCTACTACGGCGAGAAAACCTA |
| | | CTACAACCCCAGCCTGAAGTCCCGGGCCACCATCAGCATCGATACCAGCAAGAGC |
| | | CAGTTCTCCCTGAAGCTGAGCAGCGTGACAGCCGCCGATACAGCCGTGTACTACT |
| | | GTCCTAGAGGCCCCACCATGATCCGGGGCGTGATAGATTCTTGGGGCCAGGGCAC |
| | | ACTGGTCACCGTTTCTTCTGAGCCGAAAtcctccGATAAAACGCATACAtccCCG |
| | | CCATCACCAGCGCCAGAACTGAACAACAGCTCCGGCGAAATGGACCCTATCCTGC |
| | | TGACAATCAGCATCCTGAGCTTTTTCAGCGTGGCCCTGCTGGTCATCCTGGCCTG |
| | | TGTGCTGTGGAAGAAGCGGATCAAGCCCGCCGAGACAGACCAGCTGGAAGATGAG |
| | | AAGTCTGCCCTCCAGACCGAGATCGCCAACCTGCTGAAAGAGAAAGAGAAGCTGA |
| | | TCGTGTGGCCCAGCCTGCCTGACCACAAGAAAACCCTGGAACACCTGTGCAAGAA |
| | | GCCCCGGAAGAACCTGAACGTGTCCTTCAATCCCGAGAGCTTCCTGGACTGCCAG |
| | | ATCCACAGAGTGGACGACATCCAGGCCAGGGACGAAGTGGAAGGCTTCCTCCAGG |
| | | ACACATTCCCTCAGCAGCTGGAAGAGAGCGAGAAGCAGAGACTCGGCGGAGATGT |
| | | GCAGAGCCCTAATTGCCCTAGCGAGGACGTGGTCATCACCCCTGAGAGCTTCGGC |
| | | AGAGATAGCAGCCTGACATGTCTGGCCGGCAATGTGTCCGCCTGTGATGCCCCTA |
| | | TCCTGAGCAGTAGCAGAAGCCTGGATTGCAGAGAGAGCGGCAAGAACGGCCCTCA |
| | | CGTGTACCAGGATCTGCTGCTGTCTCTGGGCACCACCAATAGCACACTGCCTCCA |
| | | CCTTTCAGCCTGCAAAGCGGCATCCTGACACTGAACCCTGTGGCTCAGGGACAGC |
| | | CAATCCTGACAAGCCTGGGCAGCAATCAAGAAGAGGCCTACGTCACCATGAGCAG |
| | | CTTCTACCAGAACCAG |
| CM018 | 16 | atgggcaccagcctgctGTGCTGGATGGCCCTGTGCCTGCTGGGCGCCGACCACG |
| | | CCGATGCCTACCCCTACGACGTGCCCGACTACGCCAGCGGAGGGGGAGGCAGCCC |
| | | CACCACCACCCCAGCCCCCAGACCTCCCACCCCAGCCCCCACCATCGCCAGCCAG |
| | | CCTCTGAGCCTGAGACCCGAGGCCTGCCGCCCAGCgGCCGGCGGCGCCGTGCACA |
| | | CCAGAGGCCTGGATTTCGCCTGCGATATCTACATCTGGGCCCCACTcGCCGGCAC |
| | | CTGTGGCGTGCTGCTGCTGAGCCTGGTGATCACCCTGTACTGCAACCACCGCAAC |
| | | CGCAGGCGCGTGTGCAAGTGCCCCAGGCCCGTcGTGGAAGGCAGAGGCAGCCTGC |
| | | TTACATGTGGCGACGTGGAAGAGAACCCCGGACCTATGGGCTGGAGCTGCATCAT |
| | | CCTGTTCCTGGTGGCCACCGCCACCGGCGTGCACAGCGAGATCGTGCTGACACAG |
| | | TCTCCCGCCACACTGTCACTGTCTCCAGGCGAAAGAGCCACACTGAGCTGTAGAG |
| | | CCTCTCAGAGCGTGCGGTCTTACCTGGCCTGGTATCAGCAGAAGCCTGGACAGGC |
| | | TCCCCGGCTGCTGATCTACGATGCCAGCAATAGAGCCACAGGCATCCCCGCCAGA |
| | | TTTTCTGGCTCTGGCAGCGGCACCGATTTCACCCTGACCATAAGCAGCCTGGAAC |
| | | CTGAGGACTTCGCCGTGTACTACTGCCAGCAGAGAAGCAACTGGCCTCCTACCTT |
| | | TGGCTGCGGCACCAAGGTGGAAATCAAGGAGCCCAAGAGCAGCGACAAGACCCAC |
| | | ACCAGCCCCCCCAGCCCCGCCCCCGAGCTGAACAACAGCTCCGGCGAAATGGACC |
| | | CTATCCTGCTGACAATCAGCATCCTGAGCTTTTTCAGCGTGGCCCTGCTGGTCAT |
| | | CCTGGCCTGTGTGCTGTGGAAGAAGCGGATCAAGCCCGCCGAGACAGACCAGCTG |
| | | GAAGATGAGAAGTCTGCCCTCCAGACCGAGATCGCCAACCTGCTGAAAGAGAAAG |
| | | AGAAGCTGATCGTGTGGCCCAGCCTGCCTGACCACAAGAAAACCCTGGAACACCT |
| | | GTGCAAGAAGCCCCGGAAGAACCTGAACGTGTCCTTCAATCCCGAGAGCTTCCTG |
| | | GACTGCCAGATCCACAGAGTGGACGACATCCAGGCCAGGGACGAAGTGGAAGGCT |
| | | TCCTCCAGGACACATTCCCTCAGCAGCTGGAAGAGAGCGAGAAGCAGAGACTCGG |
| | | CGGAGATGTGCAGAGCCCTAATTGCCCTAGCGAGGACGTGGTCATCACCCCTGAG |
| | | AGCTTCGGCAGAGATAGCAGCCTGACATGTCTGGCCGGCAATGTGTCCGCCTGTG |
| | | ATGCCCCTATCCTGAGCAGTAGCAGAAGCCTGGATTGCAGAGAGAGCGGCAAGAA |
| | | CGGCCCTCACGTGTACCAGGATCTGCTGCTGTCTCTGGGCACCACCAATAGCACA |
| | | CTGCCTCCACCTTTCAGCCTGCAAAGCGGCATCCTGACACTGAACCCTGTGGCTC |
| | | AGGGACAGCCAATCCTGACAAGCCTGGGCAGCAATCAAGAAGAGGCCTACGTCAC |
| | | CATGAGCAGCTTCTACCAGAACCAGGCCACCAATTTCAGCCTGCTGAAACAGGCT |
| | | GGCGACGTGGAAGAGAACCCTGGACCTATGGAGACCGACACCCTGATCCTGTGGG |
| | | TGCTGCTGCTGCTGGTGCCCGGCAGCACCGGCCAGCTCCAGGTGCAAGAGTCTGG |
| | | ACCTGGCCTGGTCAAGCCTAGCGAGACACTGAGCCTGACCTGTACAGTGTCTGGC |
| | | GGCAGCATCAGCAACAGCTACTTTAGCTGGGGCTGGATCAGACAGCCTCCTGGCA |
| | | AGTGCCTGGAATGGATCGGCAGCTTCTACTACGGCGAGAAAACCTACTACAACCC |
| | | CAGCCTGAAGTCCCGGGCCACCATCAGCATCGATACCAGCAAGAGCCAGTTCTCC |
| | | CTGAAGCTGAGCAGCGTGACAGCCGCCGATACAGCCGTGTACTACTGTCCTAGAG |
| | | GCCCCACCATGATCCGGGGCGTGATAGATTCTTGGGGCCAGGGCACACTGGTCAC |
| | | CGTTTCTTCTGAGCCGAAAtcctccGATAAAACGCATACAtccCCGCCATCACCA |
| | | GCGCCAGAACTGAACACCAGCAAGGAAAACCCCTTTCTGTTCGCCCTGGAGGCCG |
| | | TGGTTATCAGCGTGGGCTCTATGGGCCTGATCATCAGCCTGCTGTGCGTGTACTT |
| | | CTGGCTGGAACGGACCATGCCTAGAGAGAAAGTCAAGACCCTGAAGGCCCAGAAC |
| | | AGCGAGCTGGCCTCTACCGCCAACATGCTGAGAGAACAGGTGGCCCAACTGATCC |
| | | CCACACTGAAGAACCTTGAGGACCTGGTCACCGAGTACCACGGCAACTTTTCTGC |
| | | TTGGAGCGGCGTGTCCAAAGGCCTGGCCGAATCTCTCCAGCCTGACTACAGCGAG |
| | | AGACTGTGCCTGGTGTCTGAGATCCCTCCTAAAGGCGGAGCCCTCGGAGAAGGAC |
| | | CTGGCGCCTCTCCATGTAATCAGCACAGCCCTTACTGGGCTCCTCCTTGCTACAC |
| | | CCTGAAGCCTGAGACA |
| CM019 | 17 | atgggcaccagcctgctGTGCTGGATGGCCCTGTGCCTGCTGGGCGCCGACCACG |
| | | CCGATGCCTACCCCTACGACGTGCCCGACTACGCCAGCGGAGGGGGAGGCAGCCC |
| | | CACCACCACCCCAGCCCCCAGACCTCCCACCCCAGCCCCCACCATCGCCAGCCAG |
| | | CCTCTGAGCCTGAGACCCGAGGCCTGCCGCCCAGCgGCCGGCGGCGCCGTGCACA |
| | | CCAGAGGCCTGGATTTCGCCTGCGATATCTACATCTGGGCCCCACTcGCCGGCAC |
| | | CTGTGGCGTGCTGCTGCTGAGCCTGGTGATCACCCTGTACTGCAACCACCGCAAC |
| | | CGCAGGCGCGTGTGCAAGTGCCCCAGGCCCGTcGTGGAAGGCAGAGGCAGCCTGC |
| | | TTACATGTGGCGACGTGGAAGAGAACCCCGGACCTATGGGCTGGAGCTGCATCAT |
| | | CCTGTTCCTGGTGGCCACCGCCACCGGCGTGCACAGCGAGATCGTGCTGACACAG |
| | | TCTCCCGCCACACTGTCACTGTCTCCAGGCGAAAGAGCCACACTGAGCTGTAGAG |
| | | CCTCTCAGAGCGTGCGGTCTTACCTGGCCTGGTATCAGCAGAAGCCTGGACAGGC |
| | | TCCCCGGCTGCTGATCTACGATGCCAGCAATAGAGCCACAGGCATCCCCGCCAGA |
| | | TTTTCTGGCTCTGGCAGCGGCACCGATTTCACCCTGACCATAAGCAGCCTGGAAC |
| | | CTGAGGACTTCGCCGTGTACTACTGCCAGCAGAGAAGCAACTGGCCTCCTACCTT |
| | | TGGCTGCGGCACCAAGGTGGAAATCAAGGAGCCCAAGAGCAGCGACAAGACCCAC |
| | | ACCAGCCCCCCCAGCCCCGCCCCCGAGCTGAACGGCATTTGGAGCGAGTGGAGCG |
| | | AAGCCAGAAGCTGGGATACCGAGAGCGTGCTGCCTATGTGGGTGCTCGCCCTGAT |
| | | CGTGATCTTCCTGACAATCGCCGTGCTGCTGGCCCTGAGATTCTGTGGCATCTAC |
| | | GGCTACAGACTGCGGCGGAAGTGGGAAGAGAAGATCCCCAATCCTAGCAAGAGCC |
| | | ACCTGTTCCAGAATGGCTCTGCCGAGCTTTGGCCTCCTGGCAGCATGAGCGCCTT |
| | | TACATCTGGCAGCCCTCCACATCAAGGCCCCTGGGGAAGCAGATTCCCTGAACTG |
| | | GAAGGCGTGTTCCCTGTCGGCTTCGGCGATAGCGAAGTGTCCCCTCTGACCATCG |
| | | AGGACCCTAAGCACGTGTGCGATCCTCCAAGCGGCCCTGATACAACACCAGCCGC |
| | | AAGCGATCTGCCTACCGAGCAACCTCCTAGTCCTCAGCCTGGACCTCCTGCCGCT |
| | | TCTCACACACCTGAGAAACAGGCCAGCTCCTTCGACTTCAACGGCCCCTATCTGG |
| | | GCCCTCCTCATAGCAGAAGCCTGCCTGACATTCTGGGCCAGCCTGAGCCTCCTCA |
| | | AGAAGGCGGATCTCAGAAGTCTCCTCCACCTGGCAGCCTGGAATACCTGTGTTTG |
| | | CCTGCTGGTGGCCAGGTGCAGCTGGTTCCTCTTGCTCAAGCTATGGGACCTGGAC |
| | | AGGCCGTGGAAGTGGAAAGAAGGCCATCTCAAGGCGCCGCTGGCTCTCCATCTCT |
| | | TGAATCTGGCGGAGGACCTGCTCCTCCTGCTCTGGGACCTAGAGTTGGAGGCCAG |
| | | GACCAGAAAGACAGCCCCGTGGCCATTCCTATGAGCAGCGGAGATACCGAAGATC |
| | | CCGGCGTGGCCTCTGGCTATGTGTCATCTGCCGACCTGGTGTTCACCCCTAATAG |
| | | CGGCGCTAGCTCCGTGTCTCTGGTGCCTAGTCTTGGCCTGCCTAGCGATCAGACC |
| | | CCTTCTCTGTGTCCTGGACTGGCTTCTGGACCACCTGGTGCTCCAGGACCTGTGA |
| | | AGTCTGGCTTTGAGGGCTACGTGGAACTGCCTCCTATCGAGGGCAGAAGCCCTAG |
| | | AAGCCCCAGAAACAACCCTGTGCCTCCTGAGGCCAAGTCTCCCGTTCTGAACCCT |
| | | GGCGAAAGACCCGCCGATGTGTCTCCTACATCTCCTCAGCCAGAGGGACTGCTGG |
| | | TGCTGCAACAAGTGGGCGACTACTGCTTCCTGCCTGGACTTGGACCAGGACCTCT |
| | | GAGCCTGAGAAGCAAGCCTAGCTCTCCTGGACCTGGGCCTGAGATCAAGAATCTG |
| | | GACCAGGCCTTCCAAGTGAAGAAGCCTCCAGGCCAGGCTGTGCCTCAGGTGCCAG |
| | | TGATCCAGCTGTTCAAGGCCCTGAAGCAGCAGGACTACCTGAGCCTGCCACCTTG |
| | | GGAAGTGAACAAGCCAGGCGAAGTGTGCGCCACCAATTTCAGCCTGCTGAAACAG |
| | | GCTGGCGACGTGGAAGAGAACCCTGGACCTATGGAGACCGACACCCTGATCCTGT |
| | | GGGTGCTGCTGCTGCTGGTGCCCGGCAGCACCGGCCAGCTCCAGGTGCAAGAGTC |
| | | TGGACCTGGCCTGGTCAAGCCTAGCGAGACACTGAGCCTGACCTGTACAGTGTCT |
| | | GGCGGCAGCATCAGCAACAGCTACTTTAGCTGGGGCTGGATCAGACAGCCTCCTG |
| | | GCAAGTGCCTGGAATGGATCGGCAGCTTCTACTACGGCGAGAAAACCTACTACAA |
| | | CCCCAGCCTGAAGTCCCGGGCCACCATCAGCATCGATACCAGCAAGAGCCAGTTC |
| | | TCCCTGAAGCTGAGCAGCGTGACAGCCGCCGATACAGCCGTGTACTACTGTCCTA |
| | | GAGGCCCCACCATGATCCGGGGCGTGATAGATTCTTGGGGCCAGGGCACACTGGT |
| | | CACCGTTTCTTCTGAGCCGAAAtcctccGATAAAACGCATACAtccCCGCCATCA |
| | | CCAGCGCCAGAACTGAACTGGTCCTCTTGGAGCGAGGCCATCGAGTTCGGCAGCG |
| | | ACGATGGCAATCTGGGCAGCGTGTACATCTACGTGCTGCTGATCGTGGGCACCCT |
| | | CGTGTGTGGAATCGTGCTGGGCTTCCTGTTCAAGCGGTTCCTGAGAATCCAGCGG |
| | | CTGTTCCCTCCAGTGCCTCAGATCAAGGACAAGCTGAACGACAACCACGAGGTCG |
| | | AGGACGAGATCATCTGGGAAGAGTTCACCCCTGAGGAAGGCAAGGGCTACAGAGA |
| | | AGAGGTGCTGACCGTGAAAGAGATCACC |
| CM020 | 18 | atgggcaccagcctgctGTGCTGGATGGCCCTGTGCCTGCTGGGCGCCGACCACG |
| | | CCGATGCCTACCCCTACGACGTGCCCGACTACGCCAGCGGAGGGGGAGGCAGCCC |
| | | CACCACCACCCCAGCCCCCAGACCTCCCACCCCAGCCCCCACCATCGCCAGCCAG |
| | | CCTCTGAGCCTGAGACCCGAGGCCTGCCGCCCAGCgGCCGGCGGCGCCGTGCACA |
| | | CCAGAGGCCTGGATTTCGCCTGCGATATCTACATCTGGGCCCCACTcGCCGGCAC |
| | | CTGTGGCGTGCTGCTGCTGAGCCTGGTGATCACCCTGTACTGCAACCACCGCAAC |
| | | CGCAGGCGCGTGTGCAAGTGCCCCAGGCCCGTcGTGGAAGGCAGAGGCAGCCTGC |
| | | TTACATGTGGCGACGTGGAAGAGAACCCCGGACCTATGGGCTGGAGCTGCATCAT |
| | | CCTGTTCCTGGTGGCCACCGCCACCGGCGTGCACAGCGAGATCGTGCTGACACAG |
| | | TCTCCCGCCACACTGTCACTGTCTCCAGGCGAAAGAGCCACACTGAGCTGTAGAG |
| | | CCTCTCAGAGCGTGCGGTCTTACCTGGCCTGGTATCAGCAGAAGCCTGGACAGGC |
| | | TCCCCGGCTGCTGATCTACGATGCCAGCAATAGAGCCACAGGCATCCCCGCCAGA |
| | | TTTTCTGGCTCTGGCAGCGGCACCGATTTCACCCTGACCATAAGCAGCCTGGAAC |
| | | CTGAGGACTTCGCCGTGTACTACTGCCAGCAGAGAAGCAACTGGCCTCCTACCTT |
| | | TGGCTGCGGCACCAAGGTGGAAATCAAGGAGCCCAAGAGCAGCGACAAGACCCAC |
| | | ACCAGCCCCCCCAGCCCCGCCCCCGAGCTGAACTGGTCCTCTTGGAGCGAGGCCA |
| | | TCGAGTTCGGCAGCGACGATGGCAATCTGGGCAGCGTGTACATCTACGTGCTGCT |
| | | GATCGTGGGCACCCTCGTGTGTGGAATCGTGCTGGGCTTCCTGTTCAAGCGGTTC |
| | | CTGAGAATCCAGCGGCTGTTCCCTCCAGTGCCTCAGATCAAGGACAAGCTGAACG |
| | | ACAACCACGAGGTCGAGGACGAGATCATCTGGGAAGAGTTCACCCCTGAGGAAGG |
| | | CAAGGGCTACAGAGAAGAGGTGCTGACCGTGAAAGAGATCACCGCCACCAATTTC |
| | | AGCCTGCTGAAACAGGCTGGCGACGTGGAAGAGAACCCTGGACCTATGGAGACCG |
| | | ACACCCTGATCCTGTGGGTGCTGCTGCTGCTGGTGCCCGGCAGCACCGGCCAGCT |
| | | CCAGGTGCAAGAGTCTGGACCTGGCCTGGTCAAGCCTAGCGAGACACTGAGCCTG |
| | | ACCTGTACAGTGTCTGGCGGCAGCATCAGCAACAGCTACTTTAGCTGGGGCTGGA |
| | | TCAGACAGCCTCCTGGCAAGTGCCTGGAATGGATCGGCAGCTTCTACTACGGCGA |
| | | GAAAACCTACTACAACCCCAGCCTGAAGTCCCGGGCCACCATCAGCATCGATACC |
| | | AGCAAGAGCCAGTTCTCCCTGAAGCTGAGCAGCGTGACAGCCGCCGATACAGCCG |
| | | TGTACTACTGTCCTAGAGGCCCCACCATGATCCGGGGCGTGATAGATTCTTGGGG |
| | | CCAGGGCACACTGGTCACCGTTTCTTCTGAGCCGAAAtcctccGATAAAACGCAT |
| | | ACAtccCCGCCATCACCAGCGCCAGAACTGAACGGCATTTGGAGCGAGTGGAGCG |
| | | AAGCCAGAAGCTGGGATACCGAGAGCGTGCTGCCTATGTGGGTGCTCGCCCTGAT |
| | | CGTGATCTTCCTGACAATCGCCGTGCTGCTGGCCCTGAGATTCTGTGGCATCTAC |
| | | GGCTACAGACTGCGGCGGAAGTGGGAAGAGAAGATCCCCAATCCTAGCAAGAGCC |
| | | ACCTGTTCCAGAATGGCTCTGCCGAGCTTTGGCCTCCTGGCAGCATGAGCGCCTT |
| | | TACATCTGGCAGCCCTCCACATCAAGGCCCCTGGGGAAGCAGATTCCCTGAACTG |
| | | GAAGGCGTGTTCCCTGTCGGCTTCGGCGATAGCGAAGTGTCCCCTCTGACCATCG |
| | | AGGACCCTAAGCACGTGTGCGATCCTCCAAGCGGCCCTGATACAACACCAGCCGC |
| | | AAGCGATCTGCCTACCGAGCAACCTCCTAGTCCTCAGCCTGGACCTCCTGCCGCT |
| | | TCTCACACACCTGAGAAACAGGCCAGCTCCTTCGACTTCAACGGCCCCTATCTGG |
| | | GCCCTCCTCATAGCAGAAGCCTGCCTGACATTCTGGGCCAGCCTGAGCCTCCTCA |
| | | AGAAGGCGGATCTCAGAAGTCTCCTCCACCTGGCAGCCTGGAATACCTGTGTTTG |
| | | CCTGCTGGTGGCCAGGTGCAGCTGGTTCCTCTTGCTCAAGCTATGGGACCTGGAC |
| | | AGGCCGTGGAAGTGGAAAGAAGGCCATCTCAAGGCGCCGCTGGCTCTCCATCTCT |
| | | TGAATCTGGCGGAGGACCTGCTCCTCCTGCTCTGGGACCTAGAGTTGGAGGCCAG |
| | | GACCAGAAAGACAGCCCCGTGGCCATTCCTATGAGCAGCGGAGATACCGAAGATC |
| | | CCGGCGTGGCCTCTGGCTATGTGTCATCTGCCGACCTGGTGTTCACCCCTAATAG |
| | | CGGCGCTAGCTCCGTGTCTCTGGTGCCTAGTCTTGGCCTGCCTAGCGATCAGACC |
| | | CCTTCTCTGTGTCCTGGACTGGCTTCTGGACCACCTGGTGCTCCAGGACCTGTGA |
| | | AGTCTGGCTTTGAGGGCTACGTGGAACTGCCTCCTATCGAGGGCAGAAGCCCTAG |
| | | AAGCCCCAGAAACAACCCTGTGCCTCCTGAGGCCAAGTCTCCCGTTCTGAACCCT |
| | | GGCGAAAGACCCGCCGATGTGTCTCCTACATCTCCTCAGCCAGAGGGACTGCTGG |
| | | TGCTGCAACAAGTGGGCGACTACTGCTTCCTGCCTGGACTTGGACCAGGACCTCT |
| | | GAGCCTGAGAAGCAAGCCTAGCTCTCCTGGACCTGGGCCTGAGATCAAGAATCTG |
| | | GACCAGGCCTTCCAAGTGAAGAAGCCTCCAGGCCAGGCTGTGCCTCAGGTGCCAG |
| | | TGATCCAGCTGTTCAAGGCCCTGAAGCAGCAGGACTACCTGAGCCTGCCACCTTG |
| | | GGAAGTGAACAAGCCAGGCGAAGTGTGC |
| CM021 | 19 | atgggcaccagcctgctGTGCTGGATGGCCCTGTGCCTGCTGGGCGCCGACCACG |
| | | CCGATGCCTACCCCTACGACGTGCCCGACTACGCCAGCGGAGGGGGAGGCAGCCC |
| | | CACCACCACCCCAGCCCCCAGACCTCCCACCCCAGCCCCCACCATCGCCAGCCAG |
| | | CCTCTGAGCCTGAGACCCGAGGCCTGCCGCCCAGCgGCCGGCGGCGCCGTGCACA |
| | | CCAGAGGCCTGGATTTCGCCTGCGATATCTACATCTGGGCCCCACTcGCCGGCAC |
| | | CTGTGGCGTGCTGCTGCTGAGCCTGGTGATCACCCTGTACTGCAACCACCGCAAC |
| | | CGCAGGCGCGTGTGCAAGTGCCCCAGGCCCGTcGTGGAAGGCAGAGGCAGCCTGC |
| | | TTACATGTGGCGACGTGGAAGAGAACCCCGGACCTATGGGCTGGAGCTGCATCAT |
| | | CCTGTTCCTGGTGGCCACCGCCACCGGCGTGCACAGCGAGATCGTGCTGACACAG |
| | | TCTCCCGCCACACTGTCACTGTCTCCAGGCGAAAGAGCCACACTGAGCTGTAGAG |
| | | CCTCTCAGAGCGTGCGGTCTTACCTGGCCTGGTATCAGCAGAAGCCTGGACAGGC |
| | | TCCCCGGCTGCTGATCTACGATGCCAGCAATAGAGCCACAGGCATCCCCGCCAGA |
| | | TTTTCTGGCTCTGGCAGCGGCACCGATTTCACCCTGACCATAAGCAGCCTGGAAC |
| | | CTGAGGACTTCGCCGTGTACTACTGCCAGCAGAGAAGCAACTGGCCTCCTACCTT |
| | | TGGCTGCGGCACCAAGGTGGAAATCAAGGAGCCCAAGAGCAGCGACAAGACCCAC |
| | | ACCAGCCCCCCCAGCCCCGCCCCCGAGCTGAACGGCATTTGGAGCGAGTGGAGCG |
| | | AAGCCAGAAGCTGGGATACCGAGAGCGTGCTGCCTATGTGGGTGCTCGCCCTGAT |
| | | CGTGATCTTCCTGACAATCGCCGTGCTGCTGGCCCTGAGATTCTGTGGCATCTAC |
| | | GAGAAAGTCAAGACCCTGAAGGCCCAGAACAGCGAGCTGGCCTCTACCGCCAACA |
| | | TGCTGAGAGAACAGGTGGCCCAACTGGGCTACAGACTGCGGCGGAAGTGGGAAGA |
| | | GAAGATCCCCAATCCTAGCAAGAGCCACCTGTTCCAGAATGGCTCTGCCGAGCTT |
| | | TGGCCTCCTGGCAGCATGAGCGCCTTTACATCTGGCAGCCCTCCACATCAAGGCC |
| | | CCTGGGGAAGCAGATTCCCTGAACTGGAAGGCGTGTTCCCTGTCGGCTTCGGCGA |
| | | TAGCGAAGTGTCCCCTCTGACCATCGAGGACCCTAAGCACGTGTGCGATCCTCCA |
| | | AGCGGCCCTGATACAACACCAGCCGCAAGCGATCTGCCTACCGAGCAACCTCCTA |
| | | GTCCTCAGCCTGGACCTCCTGCCGCTTCTCACACACCTGAGAAACAGGCCAGCTC |
| | | CTTCGACTTCAACGGCCCCTATCTGGGCCCTCCTCATAGCAGAAGCCTGCCTGAC |
| | | ATTCTGGGCCAGCCTGAGCCTCCTCAAGAAGGCGGATCTCAGAAGTCTCCTCCAC |
| | | CTGGCAGCCTGGAATACCTGTGTTTGCCTGCTGGTGGCCAGGTGCAGCTGGTTCC |
| | | TCTTGCTCAAGCTATGGGACCTGGACAGGCCGTGGAAGTGGAAAGAAGGCCATCT |
| | | CAAGGCGCCGCTGGCTCTCCATCTCTTGAATCTGGCGGAGGACCTGCTCCTCCTG |
| | | CTCTGGGACCTAGAGTTGGAGGCCAGGACCAGAAAGACAGCCCCGTGGCCATTCC |
| | | TATGAGCAGCGGAGATACCGAAGATCCCGGCGTGGCCTCTGGCTATGTGTCATCT |
| | | GCCGACCTGGTGTTCACCCCTAATAGCGGCGCTAGCTCCGTGTCTCTGGTGCCTA |
| | | GTCTTGGCCTGCCTAGCGATCAGACCCCTTCTCTGTGTCCTGGACTGGCTTCTGG |
| | | ACCACCTGGTGCTCCAGGACCTGTGAAGTCTGGCTTTGAGGGCTACGTGGAACTG |
| | | CCTCCTATCGAGGGCAGAAGCCCTAGAAGCCCCAGAAACAACCCTGTGCCTCCTG |
| | | AGGCCAAGTCTCCCGTTCTGAACCCTGGCGAAAGACCCGCCGATGTGTCTCCTAC |
| | | ATCTCCTCAGCCAGAGGGACTGCTGGTGCTGCAACAAGTGGGCGACTACTGCTTC |
| | | CTGCCTGGACTTGGACCAGGACCTCTGAGCCTGAGAAGCAAGCCTAGCTCTCCTG |
| | | GACCTGGGCCTGAGATCAAGAATCTGGACCAGGCCTTCCAAGTGAAGAAGCCTCC |
| | | AGGCCAGGCTGTGCCTCAGGTGCCAGTGATCCAGCTGTTCAAGGCCCTGAAGCAG |
| | | CAGGACTACCTGAGCCTGCCACCTTGGGAAGTGAACAAGCCAGGCGAAGTGTGCG |
| | | CCACCAATTTCAGCCTGCTGAAACAGGCTGGCGACGTGGAAGAGAACCCTGGACC |
| | | TATGGAGACCGACACCCTGATCCTGTGGGTGCTGCTGCTGCTGGTGCCCGGCAGC |
| | | ACCGGCCAGCTCCAGGTGCAAGAGTCTGGACCTGGCCTGGTCAAGCCTAGCGAGA |
| | | CACTGAGCCTGACCTGTACAGTGTCTGGCGGCAGCATCAGCAACAGCTACTTTAG |
| | | CTGGGGCTGGATCAGACAGCCTCCTGGCAAGTGCCTGGAATGGATCGGCAGCTTC |
| | | TACTACGGCGAGAAAACCTACTACAACCCCAGCCTGAAGTCCCGGGCCACCATCA |
| | | GCATCGATACCAGCAAGAGCCAGTTCTCCCTGAAGCTGAGCAGCGTGACAGCCGC |
| | | CGATACAGCCGTGTACTACTGTCCTAGAGGCCCCACCATGATCCGGGGCGTGATA |
| | | GATTCTTGGGGCCAGGGCACACTGGTCACCGTTTCTTCTGAGCCGAAAtectccG |
| | | ATAAAACGCATACAtccCCGCCATCACCAGCGCCAGAACTGAACTGGTCCTCTTG |
| | | GAGCGAGGCCATCGAGTTCGGCAGCGACGATGGCAATCTGGGCAGCGTGTACATC |
| | | TACGTGCTGCTGATCGTGGGCACCCTCGTGTGTGGAATCGTGCTGGGCTTCCTGT |
| | | TCAAGCGGTTCCTGAGAGCCGAGACAGACCAGCTGGAAGATGAGAAGTCTGCCCT |
| | | CCAGACCGAGATCGCCAACCTGCTGAAAGAGAAAGAGAAGCTGATCCAGCGGCTG |
| | | TTCCCTCCAGTGCCTCAGATCAAGGACAAGCTGAACGACAACCACGAGGTCGAGG |
| | | ACGAGATCATCTGGGAAGAGTTCACCCCTGAGGAAGGCAAGGGCTACAGAGAAGA |
| | | GGTGCTGACCGTGAAAGAGATCACC |
| CM022 | 20 | atgggcaccagcctgctGTGCTGGATGGCCCTGTGCCTGCTGGGCGCCGACCACG |
| | | CCGATGCCTACCCCTACGACGTGCCCGACTACGCCAGCGGAGGGGGAGGCAGCCC |
| | | CACCACCACCCCAGCCCCCAGACCTCCCACCCCAGCCCCCACCATCGCCAGCCAG |
| | | CCTCTGAGCCTGAGACCCGAGGCCTGCCGCCCAGCgGCCGGCGGCGCCGTGCACA |
| | | CCAGAGGCCTGGATTTCGCCTGCGATATCTACATCTGGGCCCCACTcGCCGGCAC |
| | | CTGTGGCGTGCTGCTGCTGAGCCTGGTGATCACCCTGTACTGCAACCACCGCAAC |
| | | CGCAGGCGCGTGTGCAAGTGCCCCAGGCCCGTcGTGGAAGGCAGAGGCAGCCTGC |
| | | TTACATGTGGCGACGTGGAAGAGAACCCCGGACCTATGGGCTGGAGCTGCATCAT |
| | | CCTGTTCCTGGTGGCCACCGCCACCGGCGTGCACAGCGAGATCGTGCTGACACAG |
| | | TCTCCCGCCACACTGTCACTGTCTCCAGGCGAAAGAGCCACACTGAGCTGTAGAG |
| | | CCTCTCAGAGCGTGCGGTCTTACCTGGCCTGGTATCAGCAGAAGCCTGGACAGGC |
| | | TCCCCGGCTGCTGATCTACGATGCCAGCAATAGAGCCACAGGCATCCCCGCCAGA |
| | | TTTTCTGGCTCTGGCAGCGGCACCGATTTCACCCTGACCATAAGCAGCCTGGAAC |
| | | CTGAGGACTTCGCCGTGTACTACTGCCAGCAGAGAAGCAACTGGCCTCCTACCTT |
| | | TGGCTGCGGCACCAAGGTGGAAATCAAGGAGCCCAAGAGCAGCGACAAGACCCAC |
| | | ACCAGCCCCCCCAGCCCCGCCCCCGAGCTGAACTGGTCCTCTTGGAGCGAGGCCA |
| | | TCGAGTTCGGCAGCGACGATGGCAATCTGGGCAGCGTGTACATCTACGTGCTGCT |
| | | GATCGTGGGCACCCTCGTGTGTGGAATCGTGCTGGGCTTCCTGTTCAAGCGGTTC |
| | | CTGAGAGCCGAGACAGACCAGCTGGAAGATGAGAAGTCTGCCCTCCAGACCGAGA |
| | | TCGCCAACCTGCTGAAAGAGAAAGAGAAGCTGATCCAGCGGCTGTTCCCTCCAGT |
| | | GCCTCAGATCAAGGACAAGCTGAACGACAACCACGAGGTCGAGGACGAGATCATC |
| | | TGGGAAGAGTTCACCCCTGAGGAAGGCAAGGGCTACAGAGAAGAGGTGCTGACCG |
| | | TGAAAGAGATCACCGCCACCAATTTCAGCCTGCTGAAACAGGCTGGCGACGTGGA |
| | | AGAGAACCCTGGACCTATGGAGACCGACACCCTGATCCTGTGGGTGCTGCTGCTG |
| | | CTGGTGCCCGGCAGCACCGGCCAGCTCCAGGTGCAAGAGTCTGGACCTGGCCTGG |
| | | TCAAGCCTAGCGAGACACTGAGCCTGACCTGTACAGTGTCTGGCGGCAGCATCAG |
| | | CAACAGCTACTTTAGCTGGGGCTGGATCAGACAGCCTCCTGGCAAGTGCCTGGAA |
| | | TGGATCGGCAGCTTCTACTACGGCGAGAAAACCTACTACAACCCCAGCCTGAAGT |
| | | CCCGGGCCACCATCAGCATCGATACCAGCAAGAGCCAGTTCTCCCTGAAGCTGAG |
| | | CAGCGTGACAGCCGCCGATACAGCCGTGTACTACTGTCCTAGAGGCCCCACCATG |
| | | ATCCGGGGCGTGATAGATTCTTGGGGCCAGGGCACACTGGTCACCGTTTCTTCTG |
| | | AGCCGAAAtcctccGATAAAACGCATACAtccCCGCCATCACCAGCGCCAGAACT |
| | | GAACGGCATTTGGAGCGAGTGGAGCGAAGCCAGAAGCTGGGATACCGAGAGCGTG |
| | | CTGCCTATGTGGGTGCTCGCCCTGATCGTGATCTTCCTGACAATCGCCGTGCTGC |
| | | TGGCCCTGAGATTCTGTGGCATCTACGAGAAAGTCAAGACCCTGAAGGCCCAGAA |
| | | CAGCGAGCTGGCCTCTACCGCCAACATGCTGAGAGAACAGGTGGCCCAACTGGGC |
| | | TACAGACTGCGGCGGAAGTGGGAAGAGAAGATCCCCAATCCTAGCAAGAGCCACC |
| | | TGTTCCAGAATGGCTCTGCCGAGCTTTGGCCTCCTGGCAGCATGAGCGCCTTTAC |
| | | ATCTGGCAGCCCTCCACATCAAGGCCCCTGGGGAAGCAGATTCCCTGAACTGGAA |
| | | GGCGTGTTCCCTGTCGGCTTCGGCGATAGCGAAGTGTCCCCTCTGACCATCGAGG |
| | | ACCCTAAGCACGTGTGCGATCCTCCAAGCGGCCCTGATACAACACCAGCCGCAAG |
| | | CGATCTGCCTACCGAGCAACCTCCTAGTCCTCAGCCTGGACCTCCTGCCGCTTCT |
| | | CACACACCTGAGAAACAGGCCAGCTCCTTCGACTTCAACGGCCCCTATCTGGGCC |
| | | CTCCTCATAGCAGAAGCCTGCCTGACATTCTGGGCCAGCCTGAGCCTCCTCAAGA |
| | | AGGCGGATCTCAGAAGTCTCCTCCACCTGGCAGCCTGGAATACCTGTGTTTGCCT |
| | | GCTGGTGGCCAGGTGCAGCTGGTTCCTCTTGCTCAAGCTATGGGACCTGGACAGG |
| | | CCGTGGAAGTGGAAAGAAGGCCATCTCAAGGCGCCGCTGGCTCTCCATCTCTTGA |
| | | ATCTGGCGGAGGACCTGCTCCTCCTGCTCTGGGACCTAGAGTTGGAGGCCAGGAC |
| | | CAGAAAGACAGCCCCGTGGCCATTCCTATGAGCAGCGGAGATACCGAAGATCCCG |
| | | GCGTGGCCTCTGGCTATGTGTCATCTGCCGACCTGGTGTTCACCCCTAATAGCGG |
| | | CGCTAGCTCCGTGTCTCTGGTGCCTAGTCTTGGCCTGCCTAGCGATCAGACCCCT |
| | | TCTCTGTGTCCTGGACTGGCTTCTGGACCACCTGGTGCTCCAGGACCTGTGAAGT |
| | | CTGGCTTTGAGGGCTACGTGGAACTGCCTCCTATCGAGGGCAGAAGCCCTAGAAG |
| | | CCCCAGAAACAACCCTGTGCCTCCTGAGGCCAAGTCTCCCGTTCTGAACCCTGGC |
| | | GAAAGACCCGCCGATGTGTCTCCTACATCTCCTCAGCCAGAGGGACTGCTGGTGC |
| | | TGCAACAAGTGGGCGACTACTGCTTCCTGCCTGGACTTGGACCAGGACCTCTGAG |
| | | CCTGAGAAGCAAGCCTAGCTCTCCTGGACCTGGGCCTGAGATCAAGAATCTGGAC |
| | | CAGGCCTTCCAAGTGAAGAAGCCTCCAGGCCAGGCTGTGCCTCAGGTGCCAGTGA |
| | | TCCAGCTGTTCAAGGCCCTGAAGCAGCAGGACTACCTGAGCCTGCCACCTTGGGA |
| | | AGTGAACAAGCCAGGCGAAGTGTGC |
| CM023 | 21 | atgggcaccagcctgctGTGCTGGATGGCCCTGTGCCTGCTGGGCGCCGACCACG |
| | | CCGATGCCTACCCCTACGACGTGCCCGACTACGCCAGCGGAGGGGGAGGCAGCCC |
| | | CACCACCACCCCAGCCCCCAGACCTCCCACCCCAGCCCCCACCATCGCCAGCCAG |
| | | CCTCTGAGCCTGAGACCCGAGGCCTGCCGCCCAGCgGCCGGCGGCGCCGTGCACA |
| | | CCAGAGGCCTGGATTTCGCCTGCGATATCTACATCTGGGCCCCACTcGCCGGCAC |
| | | CTGTGGCGTGCTGCTGCTGAGCCTGGTGATCACCCTGTACTGCAACCACCGCAAC |
| | | CGCAGGCGCGTGTGCAAGTGCCCCAGGCCCGTcGTGGAAGGCAGAGGCAGCCTGC |
| | | TTACATGTGGCGACGTGGAAGAGAACCCCGGACCTATGGGCTGGAGCTGCATCAT |
| | | CCTGTTCCTGGTGGCCACCGCCACCGGCGTGCACAGCGAGATCGTGCTGACACAG |
| | | TCTCCCGCCACACTGTCACTGTCTCCAGGCGAAAGAGCCACACTGAGCTGTAGAG |
| | | CCTCTCAGAGCGTGCGGTCTTACCTGGCCTGGTATCAGCAGAAGCCTGGACAGGC |
| | | TCCCCGGCTGCTGATCTACGATGCCAGCAATAGAGCCACAGGCATCCCCGCCAGA |
| | | TTTTCTGGCTCTGGCAGCGGCACCGATTTCACCCTGACCATAAGCAGCCTGGAAC |
| | | CTGAGGACTTCGCCGTGTACTACTGCCAGCAGAGAAGCAACTGGCCTCCTACCTT |
| | | TGGCTGCGGCACCAAGGTGGAAATCAAGGAGCCCAAGAGCAGCGACAAGACCCAC |
| | | ACCAGCCCCCCCAGCCCCGCCCCCGAGCTGAGCCCTCTGTTTCCCGGACCTAGCA |
| | | AGCCTTTCTGGGTGCTCGTTGTTGTTGGCGGCGTGCTGGCCTGTTACAGCCTGCT |
| | | GGTTACCGTGGCCTTCATCATCTTTTGGGTCCGAAGCGCCACCAATTTCAGCCTG |
| | | CTGAAACAGGCTGGCGACGTGGAAGAGAACCCTGGACCTATGGAGACCGACACCC |
| | | TGATCCTGTGGGTGCTGCTGCTGCTGGTGCCCGGCAGCACCGGCCAGCTCCAGGT |
| | | GCAAGAGTCTGGACCTGGCCTGGTCAAGCCTAGCGAGACACTGAGCCTGACCTGT |
| | | ACAGTGTCTGGCGGCAGCATCAGCAACAGCTACTTTAGCTGGGGCTGGATCAGAC |
| | | AGCCTCCTGGCAAGTGCCTGGAATGGATCGGCAGCTTCTACTACGGCGAGAAAAC |
| | | CTACTACAACCCCAGCCTGAAGTCCCGGGCCACCATCAGCATCGATACCAGCAAG |
| | | AGCCAGTTCTCCCTGAAGCTGAGCAGCGTGACAGCCGCCGATACAGCCGTGTACT |
| | | ACTGTCCTAGAGGCCCCACCATGATCCGGGGCGTGATAGATTCTTGGGGCCAGGG |
| | | CACACTGGTCACCGTTTCTTCTGAGCCGAAAtectccGATAAAACGCATACAtcc |
| | | CCGCCATCACCAGCGCCAGAACTGTCCCCGCTCTTCCCGGGCCCCTCCAAACCGT |
| | | TTTGGGTCCTGGTGGTAGTAGGTGGTGTCTTGGCgTGcTAttccCTcCTcGTgAC |
| | | gGTcGCgTTtATtATtTTcTGGGTgCGctcc |
| CM024 | 22 | atgggcaccagcctgctGTGCTGGATGGCCCTGTGCCTGCTGGGCGCCGACCACG |
| | | CCGATGCCTACCCCTACGACGTGCCCGACTACGCCAGCGGAGGGGGAGGCAGCCC |
| | | CACCACCACCCCAGCCCCCAGACCTCCCACCCCAGCCCCCACCATCGCCAGCCAG |
| | | CCTCTGAGCCTGAGACCCGAGGCCTGCCGCCCAGCgGCCGGCGGCGCCGTGCACA |
| | | CCAGAGGCCTGGATTTCGCCTGCGATATCTACATCTGGGCCCCACTcGCCGGCAC |
| | | CTGTGGCGTGCTGCTGCTGAGCCTGGTGATCACCCTGTACTGCAACCACCGCAAC |
| | | CGCAGGCGCGTGTGCAAGTGCCCCAGGCCCGTcGTGGAAGGCAGAGGCAGCCTGC |
| | | TTACATGTGGCGACGTGGAAGAGAACCCCGGACCTATGGGCTGGAGCTGCATCAT |
| | | CCTGTTCCTGGTGGCCACCGCCACCGGCGTGCACAGCGAGATCGTGCTGACACAG |
| | | TCTCCCGCCACACTGTCACTGTCTCCAGGCGAAAGAGCCACACTGAGCTGTAGAG |
| | | CCTCTCAGAGCGTGCGGTCTTACCTGGCCTGGTATCAGCAGAAGCCTGGACAGGC |
| | | TCCCCGGCTGCTGATCTACGATGCCAGCAATAGAGCCACAGGCATCCCCGCCAGA |
| | | TTTTCTGGCTCTGGCAGCGGCACCGATTTCACCCTGACCATAAGCAGCCTGGAAC |
| | | CTGAGGACTTCGCCGTGTACTACTGCCAGCAGAGAAGCAACTGGCCTCCTACCTT |
| | | TGGCTGCGGCACCAAGGTGGAAATCAAGGAGCCCAAGAGCAGCGACAAGACCCAC |
| | | ACCAGCCCCCCCAGCCCCGCCCCCGAGCTGAACAACAGCTCCGGCGAAATGGACC |
| | | CTATCCTGCTGACAATCAGCATCCTGAGCTTTTTCAGCGTGGCCCTGCTGGTCAT |
| | | CCTGGCCTGTGTGCTGTGGAAGAAGGCCACCAATTTCAGCCTGCTGAAACAGGCT |
| | | GGCGACGTGGAAGAGAACCCTGGACCTATGGAGACCGACACCCTGATCCTGTGGG |
| | | TGCTGCTGCTGCTGGTGCCCGGCAGCACCGGCCAGCTCCAGGTGCAAGAGTCTGG |
| | | ACCTGGCCTGGTCAAGCCTAGCGAGACACTGAGCCTGACCTGTACAGTGTCTGGC |
| | | GGCAGCATCAGCAACAGCTACTTTAGCTGGGGCTGGATCAGACAGCCTCCTGGCA |
| | | AGTGCCTGGAATGGATCGGCAGCTTCTACTACGGCGAGAAAACCTACTACAACCC |
| | | CAGCCTGAAGTCCCGGGCCACCATCAGCATCGATACCAGCAAGAGCCAGTTCTCC |
| | | CTGAAGCTGAGCAGCGTGACAGCCGCCGATACAGCCGTGTACTACTGTCCTAGAG |
| | | GCCCCACCATGATCCGGGGCGTGATAGATTCTTGGGGCCAGGGCACACTGGTCAC |
| | | CGTTTCTTCTGAGCCGAAAtcctccGATAAAACGCATACAtccCCGCCATCACCA |
| | | GCGCCAGAACTGAACACCAGCAAGGAAAACCCCTTTCTGTTCGCCCTGGAGGCCG |
| | | TGGTTATCAGCGTGGGCTCTATGGGCCTGATCATCAGCCTGCTGTGCGTGTACTT |
| | | CTGGCTGGAACGG |
| CM025 | 23 | atgggcaccagcctgctGTGCTGGATGGCCCTGTGCCTGCTGGGCGCCGACCACG |
| | | CCGATGCCTACCCCTACGACGTGCCCGACTACGCCAGCGGAGGGGGAGGCAGCCC |
| | | CACCACCACCCCAGCCCCCAGACCTCCCACCCCAGCCCCCACCATCGCCAGCCAG |
| | | CCTCTGAGCCTGAGACCCGAGGCCTGCCGCCCAGCgGCCGGCGGCGCCGTGCACA |
| | | CCAGAGGCCTGGATTTCGCCTGCGATATCTACATCTGGGCCCCACTcGCCGGCAC |
| | | CTGTGGCGTGCTGCTGCTGAGCCTGGTGATCACCCTGTACTGCAACCACCGCAAC |
| | | CGCAGGCGCGTGTGCAAGTGCCCCAGGCCCGTcGTGGAAGGCAGAGGCAGCCTGC |
| | | TTACATGTGGCGACGTGGAAGAGAACCCCGGACCTATGGGCTGGAGCTGCATCAT |
| | | CCTGTTCCTGGTGGCCACCGCCACCGGCGTGCACAGCAGAACAGTGGCCGCTCCT |
| | | AGCGTGTTCATCTTCCCACCTTCCGACGAGCAGCTGAAGTCTGGCACAGCCTCTG |
| | | TCGTGTGCCTGCTGAACAACTTCTACCCCAGAGAAGCCAAGGTGCAGTGGAAGGT |
| | | GGACAACGCCCTCCAGAGCGGCAATAGCCAAGAGAGCGTGACCGAGCAGGACAGC |
| | | AAGGACTCTACCTACAGCCTGAGCAGCACCCTGACACTGAGCAAGGCCGACTACG |
| | | AGAAGCACAAAGTGTACGCCTGCGAAGTGACCCACCAGGGCCTTTCTAGCCCTGT |
| | | GACCAAGAGCTTCAACCGGGGAGAGTGCGAGCCCAAGAGCTGCGATAAGACCCAC |
| | | ACCTGTCCTCCATGTCCTGAGCCCAAGAGCAGCGACAAGACCCACACCAGCCCCC |
| | | CCAGCCCCGCCCCCGAGCTGAACGGCATTTGGAGCGAGTGGAGCGAAGCCAGAAG |
| | | CTGGGATACCGAGAGCGTGCTGCCTATGTGGGTGCTCGCCCTGATCGTGATCTTC |
| | | CTGACAATCGCCGTGCTGCTGGCCCTGAGATTCTGTGGCATCTACGGCTACAGAC |
| | | TGCGGCGGAAGTGGGAAGAGAAGATCCCCAATCCTAGCAAGAGCCACCTGTTCCA |
| | | GAATGGCTCTGCCGAGCTTTGGCCTCCTGGCAGCATGAGCGCCTTTACATCTGGC |
| | | AGCCCTCCACATCAAGGCCCCTGGGGAAGCAGATTCCCTGAACTGGAAGGCGTGT |
| | | TCCCTGTCGGCTTCGGCGATAGCGAAGTGTCCCCTCTGACCATCGAGGACCCTAA |
| | | GCACGTGTGCGATCCTCCAAGCGGCCCTGATACAACACCAGCCGCAAGCGATCTG |
| | | CCTACCGAGCAACCTCCTAGTCCTCAGCCTGGACCTCCTGCCGCTTCTCACACAC |
| | | CTGAGAAACAGGCCAGCTCCTTCGACTTCAACGGCCCCTATCTGGGCCCTCCTCA |
| | | TAGCAGAAGCCTGCCTGACATTCTGGGCCAGCCTGAGCCTCCTCAAGAAGGCGGA |
| | | TCTCAGAAGTCTCCTCCACCTGGCAGCCTGGAATACCTGTGTTTGCCTGCTGGTG |
| | | GCCAGGTGCAGCTGGTTCCTCTTGCTCAAGCTATGGGACCTGGACAGGCCGTGGA |
| | | AGTGGAAAGAAGGCCATCTCAAGGCGCCGCTGGCTCTCCATCTCTTGAATCTGGC |
| | | GGAGGACCTGCTCCTCCTGCTCTGGGACCTAGAGTTGGAGGCCAGGACCAGAAAG |
| | | ACAGCCCCGTGGCCATTCCTATGAGCAGCGGAGATACCGAAGATCCCGGCGTGGC |
| | | CTCTGGCTATGTGTCATCTGCCGACCTGGTGTTCACCCCTAATAGCGGCGCTAGC |
| | | TCCGTGTCTCTGGTGCCTAGTCTTGGCCTGCCTAGCGATCAGACCCCTTCTCTGT |
| | | GTCCTGGACTGGCTTCTGGACCACCTGGTGCTCCAGGACCTGTGAAGTCTGGCTT |
| | | TGAGGGCTACGTGGAACTGCCTCCTATCGAGGGCAGAAGCCCTAGAAGCCCCAGA |
| | | AACAACCCTGTGCCTCCTGAGGCCAAGTCTCCCGTTCTGAACCCTGGCGAAAGAC |
| | | CCGCCGATGTGTCTCCTACATCTCCTCAGCCAGAGGGACTGCTGGTGCTGCAACA |
| | | AGTGGGCGACTACTGCTTCCTGCCTGGACTTGGACCAGGACCTCTGAGCCTGAGA |
| | | AGCAAGCCTAGCTCTCCTGGACCTGGGCCTGAGATCAAGAATCTGGACCAGGCCT |
| | | TCCAAGTGAAGAAGCCTCCAGGCCAGGCTGTGCCTCAGGTGCCAGTGATCCAGCT |
| | | GTTCAAGGCCCTGAAGCAGCAGGACTACCTGAGCCTGCCACCTTGGGAAGTGAAC |
| | | AAGCCAGGCGAAGTGTGCGCCACCAATTTCAGCCTGCTGAAACAGGCTGGCGACG |
| | | TGGAAGAGAACCCTGGACCTATGGAGACCGACACCCTGATCCTGTGGGTGCTGCT |
| | | GCTGCTGGTGCCCGGCAGCACCGGCGCCTCTACAAAGGGCCCTAGTGTGTTCCCT |
| | | CTGGCTCCCAGCAGCAAGTCTACATCTGGCGGAACAGCCGCTCTGGGCTGCCTGG |
| | | TCAAGGATTACTTTCCCGAGCCTGTGACCGTGTCCTGGAATAGCGGAGCACTGAC |
| | | AAGCGGCGTGCACACCTTTCCAGCTGTGCTGCAAAGCAGCGGCCTGTACTCTCTG |
| | | AGCAGCGTGGTCACAGTGCCTAGCTCTAGCCTGGGCACCCAGACCTACATCTGCA |
| | | ATGTGAACCACAAGCCTAGCAACACCAAGGTGGACAAGAAGGTGGAACCCAAGAG |
| | | CTGCGACAAGACCCACACCTGTCCTCCATGTCCTGAGCCGAAAtectccGATAAA |
| | | ACGCATACAtccCCGCCATCACCAGCGCCAGAACTGAACTGGTCCTCTTGGAGCG |
| | | AGGCCATCGAGTTCGGCAGCGACGATGGCAATCTGGGCAGCGTGTACATCTACGT |
| | | GCTGCTGATCGTGGGCACCCTCGTGTGTGGAATCGTGCTGGGCTTCCTGTTCAAG |
| | | CGGTTCCTGAGAATCCAGCGGCTGTTCCCTCCAGTGCCTCAGATCAAGGACAAGC |
| | | TGAACGACAACCACGAGGTCGAGGACGAGATCATCTGGGAAGAGTTCACCCCTGA |
| | | GGAAGGCAAGGGCTACAGAGAAGAGGTGCTGACCGTGAAAGAGATCACC |
| CM026 | 24 | atgggcaccagcctgctGTGCTGGATGGCCCTGTGCCTGCTGGGCGCCGACCACG |
| | | CCGATGCCTACCCCTACGACGTGCCCGACTACGCCAGCGGAGGGGGAGGCAGCCC |
| | | CACCACCACCCCAGCCCCCAGACCTCCCACCCCAGCCCCCACCATCGCCAGCCAG |
| | | CCTCTGAGCCTGAGACCCGAGGCCTGCCGCCCAGCgGCCGGCGGCGCCGTGCACA |
| | | CCAGAGGCCTGGATTTCGCCTGCGATATCTACATCTGGGCCCCACTcGCCGGCAC |
| | | CTGTGGCGTGCTGCTGCTGAGCCTGGTGATCACCCTGTACTGCAACCACCGCAAC |
| | | CGCAGGCGCGTGTGCAAGTGCCCCAGGCCCGTcGTGGAAGGCAGAGGCAGCCTGC |
| | | TTACATGTGGCGACGTGGAAGAGAACCCCGGACCTATGGGCTGGAGCTGCATCAT |
| | | CCTGTTCCTGGTGGCCACCGCCACCGGCGTGCACAGCGACATCCAGATGACACAG |
| | | AGCCCTAGCAGCCTGTCTGCCAGCGTGGGAGACAGAGTGACCATCACATGCAAGG |
| | | CCAGCCAGAACGTGGGCATCAACGTGGCCTGGTATCAGCAGAAGCCTGGCAAGGC |
| | | CCCTAAGAGCCTGATCAGCAGCGCCAGCTACAGATACAGCGGCGTGCCAAGCAGA |
| | | TTTTCCGGCTCTGGCAGCGGCACCGATTTCACCCTGACAATATCCAGCCTCCAGC |
| | | CTGAGGACTTCGCCACCTACTTTTGCCAGCAGTACGACACATACCCCTTCACCTT |
| | | CGGCTGCGGCACCAAGGTGGAAATCAAGGAGCCCAAGAGCAGCGACAAGACCCAC |
| | | ACCAGCCCCCCCAGCCCCGCCCCCGAGCTGAACGGCATTTGGAGCGAGTGGAGCG |
| | | AAGCCAGAAGCTGGGATACCGAGAGCGTGCTGCCTATGTGGGTGCTCGCCCTGAT |
| | | CGTGATCTTCCTGACAATCGCCGTGCTGCTGGCCCTGAGATTCTGTGGCATCTAC |
| | | GGCTACAGACTGCGGCGGAAGTGGGAAGAGAAGATCCCCAATCCTAGCAAGAGCC |
| | | ACCTGTTCCAGAATGGCTCTGCCGAGCTTTGGCCTCCTGGCAGCATGAGCGCCTT |
| | | TACATCTGGCAGCCCTCCACATCAAGGCCCCTGGGGAAGCAGATTCCCTGAACTG |
| | | GAAGGCGTGTTCCCTGTCGGCTTCGGCGATAGCGAAGTGTCCCCTCTGACCATCG |
| | | AGGACCCTAAGCACGTGTGCGATCCTCCAAGCGGCCCTGATACAACACCAGCCGC |
| | | AAGCGATCTGCCTACCGAGCAACCTCCTAGTCCTCAGCCTGGACCTCCTGCCGCT |
| | | TCTCACACACCTGAGAAACAGGCCAGCTCCTTCGACTTCAACGGCCCCTATCTGG |
| | | GCCCTCCTCATAGCAGAAGCCTGCCTGACATTCTGGGCCAGCCTGAGCCTCCTCA |
| | | AGAAGGCGGATCTCAGAAGTCTCCTCCACCTGGCAGCCTGGAATACCTGTGTTTG |
| | | CCTGCTGGTGGCCAGGTGCAGCTGGTTCCTCTTGCTCAAGCTATGGGACCTGGAC |
| | | AGGCCGTGGAAGTGGAAAGAAGGCCATCTCAAGGCGCCGCTGGCTCTCCATCTCT |
| | | TGAATCTGGCGGAGGACCTGCTCCTCCTGCTCTGGGACCTAGAGTTGGAGGCCAG |
| | | GACCAGAAAGACAGCCCCGTGGCCATTCCTATGAGCAGCGGAGATACCGAAGATC |
| | | CCGGCGTGGCCTCTGGCTATGTGTCATCTGCCGACCTGGTGTTCACCCCTAATAG |
| | | CGGCGCTAGCTCCGTGTCTCTGGTGCCTAGTCTTGGCCTGCCTAGCGATCAGACC |
| | | CCTTCTCTGTGTCCTGGACTGGCTTCTGGACCACCTGGTGCTCCAGGACCTGTGA |
| | | AGTCTGGCTTTGAGGGCTACGTGGAACTGCCTCCTATCGAGGGCAGAAGCCCTAG |
| | | AAGCCCCAGAAACAACCCTGTGCCTCCTGAGGCCAAGTCTCCCGTTCTGAACCCT |
| | | GGCGAAAGACCCGCCGATGTGTCTCCTACATCTCCTCAGCCAGAGGGACTGCTGG |
| | | TGCTGCAACAAGTGGGCGACTACTGCTTCCTGCCTGGACTTGGACCAGGACCTCT |
| | | GAGCCTGAGAAGCAAGCCTAGCTCTCCTGGACCTGGGCCTGAGATCAAGAATCTG |
| | | GACCAGGCCTTCCAAGTGAAGAAGCCTCCAGGCCAGGCTGTGCCTCAGGTGCCAG |
| | | TGATCCAGCTGTTCAAGGCCCTGAAGCAGCAGGACTACCTGAGCCTGCCACCTTG |
| | | GGAAGTGAACAAGCCAGGCGAAGTGTGCGCCACCAATTTCAGCCTGCTGAAACAG |
| | | GCTGGCGACGTGGAAGAGAACCCTGGACCTATGGAGACCGACACCCTGATCCTGT |
| | | GGGTGCTGCTGCTGCTGGTGCCCGGCAGCACCGGCCAGGTTCAGCTGGTTCAGTC |
| | | TGGCGCCGAGGAAGTGAAGAAACCTGGCAGCTCTGTGAAGGTGTCCTGCAAGGCC |
| | | AGCGGCTACACCTTTACCACCTACTACCTGCACTGGGTCCGACAGGCTCCAGGCC |
| | | AATGTCTGGAATGGATGGGCTGGATCTACCCCGGCAATGTGCACGCCCAGTACAA |
| | | CGAGAAGTTCAAGGGCAGAGTGACCATCACCGCCGACAAGAGCACAAGCACCGCC |
| | | TACATGGAACTGAGCAGCCTGAGAAGCGAGGACACCGCCGTGTACTACTGTGCCA |
| | | GAAGCTGGGAGGGCTTCCCTTATTGGGGCCAGGGAACCACCGTGACCGTTAGCTC |
| | | TGAGCCGAAAtcctccGATAAAACGCATACAtccCCGCCATCACCAGCGCCAGAA |
| | | CTGAACTGGTCCTCTTGGAGCGAGGCCATCGAGTTCGGCAGCGACGATGGCAATC |
| | | TGGGCAGCGTGTACATCTACGTGCTGCTGATCGTGGGCACCCTCGTGTGTGGAAT |
| | | CGTGCTGGGCTTCCTGTTCAAGCGGTTCCTGAGAATCCAGCGGCTGTTCCCTCCA |
| | | GTGCCTCAGATCAAGGACAAGCTGAACGACAACCACGAGGTCGAGGACGAGATCA |
| | | TCTGGGAAGAGTTCACCCCTGAGGAAGGCAAGGGCTACAGAGAAGAGGTGCTGAC |
| | | CGTGAAAGAGATCACC |

The FAB antibody fragments include two variable and two constant domains, of which both domains contained respective heavy and light chains (FIGS. 1A and 2A). The Fv fragment is only comprised of the heavy and light chains of the variable domain. In a subset of FAB antibody fragments, an additional disulfide bond (“dsFv”) is incorporated to stabilize the Fv portions.

Each construct further included several subdomains, as shown below and in Tables 4-5:
1. CM015:
   Anti-TBRII light chain (SEQ ID NO: 25)-Hinge (SEQ ID NO: 26)-Truncated IL-7 gamma extracellular domain (SEQ ID NO: 27)-IL-7 gamma transmembrane domain (SEQ ID NO: 28)-IL-7 gamma intracellular domain (SEQ ID NO: 29)-P2A (SEQ ID NO: 30)-anti-TBRII heavy chain (SEQ ID NO: 31)-Hinge (SEQ ID NO: 32)-Truncated IL-7 alpha extracellular domain (SEQ ID NO: 33)-IL-7 alpha transmembrane domain (SEQ ID NO: 34)-IL-7 alpha intracellular domain (SEQ ID NO: 35)
2. CM016:
   Anti-TBRII light chain (SEQ ID NO: 25)-Hinge (SEQ ID NO: 32)-Truncated IL-7 alpha extracellular domain (SEQ ID NO: 33)-IL-7 alpha transmembrane domain (SEQ ID NO: 34)-IL-7 alpha intracellular domain (SEQ ID NO: 35)-Hinge (SEQ ID NO: 32)-P2A (SEQ ID NO: 30)-Anti-TBRII heavy chain (SEQ ID NO: 31)-Hinge (SEQ ID NO: 26)-Truncated IL-7 gamma extracellular domain (SEQ ID NO: 27)-IL-7 gamma transmembrane domain (SEQ ID NO: 28)-IL-7 gamma transmembrane domain (SEQ ID NO: 29)
3. CM017:
   Anti-TBRII light chain (SEQ ID NO: 25)-Hinge (SEQ ID NO: 26)-Truncated IL-7 gamma extracellular domain (SEQ ID NO: 27)-IL-7 gamma transmembrane domain (SEQ ID NO: 28)-Jun leucine zipper (SEQ ID NO: 26)-IL-7 gamma intracellular domain (SEQ ID NO: 29)-P2A (SEQ ID NO: 30)-Anti-TBRII heavy chain (SEQ ID NO: 31)-Hinge (SEQ ID NO: 32)-Truncated IL-7 alpha extracellular domain (SEQ ID NO: 33)-IL-7 alpha transmembrane domain (SEQ ID NO: 34)-Fos leucine zipper (SEQ ID NO: 37)-IL-7 alpha intracellular domain (SEQ ID NO: 35)
4. CM018:
   Anti-TBRII light chain (SEQ ID NO: 25)-Hinge (SEQ ID NO: 32)-Truncated IL-7 extracellular domain (SEQ ID NO: 33)-IL-7 alpha transmembrane domain (SEQ ID NO: 34)-Jun leucine zipper (SEQ ID NO: 36)-IL-7 alpha intracellular domain (SEQ ID NO: 35)-P2A (SEQ ID NO: 30)-Anti-TBRII heavy chain (SEQ ID NO: 31)-Hinge (SEQ ID NO: 26)-Truncated IL-7 gamma extracellular domain (SEQ ID NO: 27)-IL-7 gamma transmembrane domain (SEQ ID NO: 28) Fos leucine zipper (SEQ ID NO: 37)-IL-7 gamma intracellular domain (SEQ ID NO: 29)
5. CM019:
   Anti-TBRII light chain (SEQ ID NO: 25)-Hinge (SEQ ID NO: 26)-Truncated GMCSF2Rb extracellular domain (SEQ ID NO: 38)-GMCSF2Rb transmembrane domain (SEQ ID NO: 39)-GMCSF2Rb intracellular domain (SEQ ID NO: 40)-P2A (SEQ ID NO: 30)-Anti-TBRII heavy chain (SEQ ID NO: 31)-Hinge (SEQ ID NO: 32)-Truncated GMCSF2Ra extracellular domain (SEQ ID NO: 41)-GMCSF2Ra transmembrane domain (SEQ ID NO: 42)-GMCSF2Ra intracellular domain (SEQ ID NO: 43)
6. CM020:
   Anti-TBRII light chain (SEQ ID NO: 25)-Hinge (SEQ ID NO: 26)-Truncated GMCSF2Ra extracellular domain (SEQ ID NO: 41)-GMCSF2Ra transmembrane domain (SEQ ID NO: 42)-GMCSF2Ra intracellular domain (SEQ ID NO: 43)-P2A (SEQ ID NO: 30)-Anti-TBRII heavy chain (SEQ ID NO: 31)-Hinge (SEQ ID NO: 32)-Truncated GMCSF2Rb extracellular domain (SEQ ID NO: 38)-GMCSF2Rb transmembrane domain (SEQ ID NO: 39)-GMCSF2Rb intracellular domain (SEQ ID NO: 40)
7. CM021:
   Anti-TBRII light chain (SEQ ID NO: 25)-Hinge (SEQ ID NO: 26)-Truncated GMCSF2Rb extracellular domain (SEQ ID NO: 38)-GMCSF2Rb transmembrane domain (SEQ ID NO: 39)-Jun leucine zipper (SEQ ID NO: 36)-GMCSF2Rb intracellular domain (SEQ ID NO: 40)-P2A (SEQ ID NO: 30)-Anti-TBRII heavy chain (SEQ ID NO: 31)-Hinge (SEQ ID NO: 32)-Truncated GMCSF2Ra extracellular domain (SEQ ID NO: 41)-GMCSF2Ra transmembrane domain (SEQ ID NO: 42)-Fos leucine zipper (SEQ ID NO: 37)-GMCSF2Ra intracellular domain (SEQ ID NO: 43)
8. CM022:
   Anti-TBRII light chain (SEQ ID NO: 25)-Hinge (SEQ ID NO: 26)-Truncated GMCSF2Ra extracellular domain (SEQ ID NO: 41)-GMCSF2Ra transmembrane domain (SEQ ID NO: 42)-Jun leucine zipper (SEQ ID NO: 36)-GMCSF2Ra intracellular domain (SEQ ID NO: 43)-P2A (SEQ ID NO: 30)-Anti-TBRII heavy chain (SEQ ID NO: 31)-Hinge (SEQ ID NO: 32)-Truncated GMCSF2Rb extracellular domain (SEQ ID NO: 38)-GMCSF2Rb transmembrane domain (SEQ ID NO: 39)-Fos leucine zipper (SEQ ID NO: 37)-GMCSF2Rb intracellular domain (SEQ ID NO: 40)
9. CM023:
   Anti-TBRII light chain (SEQ ID NO: 25)-Hinge (SEQ ID NO: 26)-Truncated CD28 extracellular domain (SEQ ID NO: 44)-CD28 transmembrane domain (SEQ ID NO: 45)-Truncated CD28 intracellular domain (sequence: RS)-P2A (SEQ ID NO: 30)-Anti-TBRII heavy chain (SEQ ID NO: 31)-Hinge (SEQ ID NO: 32)-Truncated CD28 extracellular domain (wobbled) (SEQ ID NO: 46)-CD28 transmembrane domain (wobbled) (SEQ ID NO: 47)-Truncated CD28 intracellular domain (wobbled) (sequence: RS)
10. CM024:
   Anti-TBRII light chain (SEQ ID NO: 25)-Hinge (SEQ ID NO: 32)-Truncated IL-7 alpha extracellular domain (SEQ ID NO: 33)-IL-7 alpha transmembrane domain (SEQ ID NO: 34)-Truncated IL-7 alpha intracellular domain (sequence: KK)-Hinge (SEQ ID NO: 32)-P2A (SEQ ID NO: 30)-Anti-TBRII heavy chain (SEQ ID NO: 31)-Hinge (SEQ ID NO: 26)-Truncated IL-7 gamma extracellular domain (SEQ ID NO: 27)-IL-7 gamma transmembrane domain (SEQ ID NO: 28)-Truncated IL-7 gamma intracellular domain (sequence: ER)
11. CM025:
   FAB light chain (SEQ ID NO: 50)-Hinge (SEQ ID NO: 26)-Truncated GMCSF2Rb extracellular domain (SEQ ID NO: 38)-GMCSF2Rb transmembrane domain (SEQ ID NO: 39)-GMCSF2Rb intracellular domain (SEQ ID NO: 40)-P2A (SEQ ID NO: 30)-FAB heavy chain (SEQ ID NO: 51)-Hinge (SEQ ID NO: 32)-Truncated GMCSF2Ra extracellular domain (SEQ ID NO: 41)-GMCSF2Ra transmembrane domain (SEQ ID NO: 42)-GMCSF2Ra intracellular domain (SEQ ID NO: 43)
12. CM026:
   Anti-BHA10 light chain (SEQ ID NO: 48)-Hinge (SEQ ID NO: 26)-Truncated GMCSF2Rb extracellular domain (SEQ ID NO: 38)-GMCSF2Rb transmembrane domain (SEQ ID NO: 39)-GMCSF2Rb intracellular domain (SEQ ID NO: 40)-P2A (SEQ ID NO: 30)-Anti-BHA10 heavy chain (SEQ ID NO: 49)-Hinge (SEQ ID NO: 32)-Truncated GMCSF2Ra extracellular domain (SEQ ID NO: 41)-GMCSF2Ra transmembrane domain (SEQ ID NO: 42)-GMCSF2Ra intracellular domain (SEQ ID NO: 43)

TABLE 4
Amino acid sequences of CM015-CM026 construct subdomains (SEQ ID NOs: 25-51).
The modified cysteine used to generate disulfide
bonds is written in bold and underlined
in SEQ ID NOs: 25 and 31.

| | SEQ ID | |
|---|---|---|
| Construct: | NO: | Sequence: |
| Anti-TBRII | 25 | EIVLTQSPATLSLSPGERATLSCRASQSVRSYLAWYQQKPGQAPRLLIY |
| Light Chain | | DASNRATGIPARFSGSGSGTDFTLTISSLEPEDFAVYYCQQRSNWPPTF |
| | | GCGTKVEIK |
| Hinge | 26 | EPKSSDKTHTSPPSPAPEL |
| Truncated IL7 | 27 | NTSKENPFLFALEA |
| gamma | | |
| extracellular | | |
| domain | | |
| IL7 gamma | 28 | VVISVGSMGLIISLLCVYFWL |
| transmembrane | | |
| domain | | |
| IL7 gamma | 29 | VVISVGSMGLIISLLCVYFWLERTMPRIPTLKNLEDLVTEYHGNFSAWS |
| intracellular | | GVSKGLAESLQPDYSERLCLVSEIPPKGGALGEGPGASPCNQHSPYWA |
| domain | | PPCYTLKPET |
| P2A | 30 | ATNFSLLKQAGDVEENPGP |
| Anti-TBRII | 31 | QLQVQESGPGLVKPSETLSLTCTVSGGSISNSYFSWGWIRQPPGKCLE |
| Heavy chain | | WIGSFYYGEKTYYNPSLKSRATISIDTSKSQFSLKLSSVTAADTAVYYC |
| | | PRGPTMIRGVIDSWGQGTLVTVSS |
| Hinge | 32 | EPKSSDKTHTSPPSPAPEL |
| Truncated IL7 | 33 | NNSSGEMD |
| alpha | | |
| extracellular | | |
| domain | | |
| IL7 alpha | 34 | PILLTISILSFFSVALLVILACVLW |
| transmembrane | | |
| domain | | |
| IL7 alpha | 35 | KKRIKPIVWPSLPDHKKTLEHLCKKPRKNLNVSFNPESFLDCQIHRVDD |
| intracellular | | IQARDEVEGFLQDTFPQQLEESEKQRLGGDVQSPNCPSEDVVITPESFG |
| domain | | RDSSLTCLAGNVSACDAPILSSSRSLDCRESGKNGPHVYQDLLLSLGTT |
| | | NSTLPPPFSLQSGILTLNPVAQGQPILTSLGSNQEEAYVTMSSFYQNQ |
| Jun Leucine | 36 | EKVKTLKAQNSELASTANMLREQVAQL |
| Zipper | | |
| Fos Leucine | 37 | AETDQLEDEKSALQTEIANLLKEKEKL |
| Zipper | | |
| Truncated | 38 | NGIWSEWSEARSWDTESVLPMW |
| GMCSF2Rb | | |
| extracellular | | |
| domain | | |
| GMCSF2Rb | 39 | VLALIVIFLTIAVLLAL |
| transmembrane | | |
| domain | | |
| GMCSF2Rb | 40 | RFCGIYGYRLRRKWEEKIPNPSKSHLFQNGSAELWPPGSMSAFTSGSPP |
| intracellular | | HQGPWGSRFPELEGVFPVGFGDSEVSPLTIEDPKHVCDPPSGPDTTPAA |
| domain | | SDLPTEQPPSPQPGPPAASHTPEKQASSFDFNGPYLGPPHSRSLPDILGQ |
| | | PEPPQEGGSQKSPPPGSLEYLCLPAGGQVQLVPLAQAMGPGQAVEVER |
| | | RPSQGAAGSPSLESGGGPAPPALGPRVGGQDQKDSPVAIPMSSGDTED |
| | | PGVASGYVSSADLVFTPNSGASSVSLVPSLGLPSDQTPSLCPGLASGPP |
| | | GAPGPVKSGFEGYVELPPIEGRSPRSPRNNPVPPEAKSPVLNPGERPAD |
| | | VSPTSPQPEGLLVLQQVGDYCFLPGLGPGPLSLRSKPSSPGPGPEIKNLD |
| | | QAFQVKKPPGQAVPQVPVIQLFKALKQQDYLSLPPWEVNKPGEVC |
| Truncated | 41 | NWSSWSEAIEFGSDDG |
| GMCSF2Ra | | |
| extracellular | | |
| domain | | |
| GMCSF2Ra | 42 | NLGSVYIYVLLIVGTLVCGIVLGFLF |
| transmembrane | | |
| domain | | |
| GMCSF2Ra | 43 | KRFLRIQRLFPPVPQIKDKLNDNHEVEDEIIWEEFTPEEGKGYREEVLTV |
| Intracellular | | KEIT |
| domain | | |
| Truncated | 44 | SPLFPGPSK |
| CD28 | | |
| extracellular | | |
| domain | | |
| CD28 | 45 | PFWVLVVVGGVLACYSLLVTVAFIIFWV |
| Transmembrane | | |
| domain | | |
| Truncated | 46 | SPLFPGPSKP |
| CD28 | | |
| extracellular | | |
| domain | | |
| (Wobbled) | | |
| CD28 | 47 | FWVLVVVGGVLACYSLLVTVAFIIFWV |
| Transmembrane | | |
| domain | | |
| (Wobbled) | | |
| Anti-BHA10 | 48 | DIQMTQSPSSLSASVGDRVTITCKASQNVGINVAWYQQKPGKAPKSLIS |
| Light chain | | SASYRYSGVPSRFSGSGSGTDFTLTISSLQPEDFATYFCQQYDTYPFTFG |
| | | CGTKVEIK |
| Anti-BHA10 | 49 | QVQLVQSGAEEVKKPGSSVKVSCKASGYTFTTYYLHWVRQAPGQCLE |
| Heavy chain | | WMGWIYPGNVHAQYNEKFKGRVTITADKSTSTAYMELSSLRSEDTAV |
| | | YYCARSWEGFPYWGQGTTVTVSS |
| Modified FAB | 50 | RTVAAPSVFIFPPSDEQLKSGTASVVCLLNNFYPREAKVQWKVDNALQ |
| Light chain | | SGNSQESVTEQDSKDSTYSLSSTLTLSKADYEKHKVYACEVTHQGLSS |
| | | PVTKSFNRGECEPKSCDKTHTCPPCP |
| Modified FAB | 51 | ASTKGPSVFPLAPSSKSTSGGTAALGCLVKDYFPEPVTVSWNSGALTS |
| Heavy chain | | GVHTFPAVLQSSGLYSLSSVVTVPSSSLGTQTYICNVNHKPSNTKVDK |
| | | KVEPKSCDKTHTCPPCP |
| Wild type FV | 79 | EIVLTQSPATLSLSPGERATLSCRASQSVRSYLAWYQQKPGQAPRLLIY |
| Light Chain | | DASNRATGIPARFSGSGSGTDFTLTISSLEPEDFAVYYCQQRSNWPPTF |
| | | GQGTKVEIK |
| Wild type FV | 80 | QLQVQESGPGLVKPSETLSLTCTVSGGSISNSYFSWGWIRQPPGKGLE |
| Heavy Chain | | WIGSFYYGEKTYYNPSLKSRATISIDTSKSQFSLKLSSVTAADTAVYYC |
| | | PRGPTMIRGVIDSWGQGTLVTVSS |
| Wild type FAB | 81 | EIVLTQSPATLSLSPGERATLSCRASQSVRSYLAWYQQKPGQAPRLLIY |
| Light Chain | | DASNRATGIPARFSGSGSGTDFTLTISSLEPEDFAVYYCQQRSNWPPTF |
| | | GQGTKVEIKRTVAAPSVFIFPPSDEQLKSGTASVVCLLNNFYPREAKVQ |
| | | WKVDNALQSGNSQESVTEQDSKDSTYSLSSTLTLSKADYEKHKVYAC |
| | | EVTHQGLSSPVTKSFNRGEC |
| Wild type FAB | 82 | QLQVQESGPGLVKPSETLSLTCTVSGGSISNSYFSWGWIRQPPGKGLE |
| Heavy Chain | | WIGSFYYGEKTYYNPSLKSRATISIDTSKSQFSLKLSSVTAADTAVYYC |
| | | PRGPTMIRGVIDSWGQGTLVTVSSASTKGPSVFPLAPSSKSTSGGTAAL |
| | | GCLVKDYFPEPVTVSWNSGALTSGVHTFPAVLQSSGLYSLSSVVTVPS |
| | | SSLGTQTYICNVNHKPSNTKVDKKVEPKSC |

TABLE 5
DNA sequences of CM015-CM026 construct subdomains (SEQ ID NOs: 52-78)

| Construct: | SEQ ID NO: | Sequence: |
|---|---|---|
| Anti-TBRII | 52 | GAGATCGTGCTGACACAGTCTCCCGCCACACTGTCACTGTCTCCA |
| Light Chain | | GGCGAAAGAGCCACACTGAGCTGTAGAGCCTCTCAGAGCGTGCG |
| | | GTCTTACCTGGCCTGGTATCAGCAGAAGCCTGGACAGGCTCCCCG |
| | | GCTGCTGATCTACGATGCCAGCAATAGAGCCACAGGCATCCCCGC |
| | | CAGATTTTCTGGCTCTGGCAGCGGCACCGATTTCACCCTGACCAT |
| | | AAGCAGCCTGGAACCTGAGGACTTCGCCGTGTACTACTGCCAGCA |
| | | GAGAAGCAACTGGCCTCCTACCTTTGGCTGCGGCACCAAGGTGG |
| | | AAATCAAG |
| Hinge | 53 | GAGCCCAAGAGCAGCGACAAGACCCACACCAGCCCCCCCAGCCC |
| | | CGCCCCCGAGCTG |
| Truncated | 54 | AACACCAGCAAGGAAAACCCCTTTCTGTTCGCCCTGGAGGCC |
| IL7 gamma | | |
| extracellular | | |
| domain | | |
| IL7 gamma | 55 | GTGGTTATCAGCGTGGGCTCTATGGGCCTGATCATCAGCCTGCTG |
| Transmembrane | | TGCGTGTACTTCTGGCTG |
| domain | | |
| IL7 gamma | 56 | GAACGGACCATGCCTAGAATCCCCACACTGAAGAACCTTGAGGA |
| intracellular | | CCTGGTCACCGAGTACCACGGCAACTTTTCTGCTTGGAGCGGCGT |
| domain | | GTCCAAAGGCCTGGCCGAATCTCTCCAGCCTGACTACAGCGAGA |
| | | GACTGTGCCTGGTGTCTGAGATCCCTCCTAAAGGCGGAGCCCTCG |
| | | GAGAAGGACCTGGCGCCTCTCCATGTAATCAGCACAGCCCTTACT |
| | | GGGCTCCTCCTTGCTACACCCTGAAGCCTGAGACA |
| P2A | 57 | GCCACCAATTTCAGCCTGCTGAAACAGGCTGGCGACGTGGAAGA |
| | | GAACCCTGGACCT |
| Anti-TBRII | 58 | CAGCTCCAGGTGCAAGAGTCTGGACCTGGCCTGGTCAAGCCTAGC |
| Heavy chain | | GAGACACTGAGCCTGACCTGTACAGTGTCTGGCGGCAGCATCAG |
| | | CAACAGCTACTTTAGCTGGGGCTGGATCAGACAGCCTCCTGGCAA |
| | | GTGCCTGGAATGGATCGGCAGCTTCTACTACGGCGAGAAAACCT |
| | | ACTACAACCCCAGCCTGAAGTCCCGGGCCACCATCAGCATCGATA |
| | | CCAGCAAGAGCCAGTTCTCCCTGAAGCTGAGCAGCGTGACAGCC |
| | | GCCGATACAGCCGTGTACTACTGTCCTAGAGGCCCCACCATGATC |
| | | CGGGGCGTGATAGATTCTTGGGGCCAGGGCACACTGGTCACCGTT |
| | | TCTTCT |
| Hinge | 59 | GAGCCGAAAtcctccGATAAAACGCATACAtccCCGCCATCACCAGCG |
| | | CCAGAACTG |
| Truncated | 60 | AACAACAGCTCCGGCGAAATGGAC |
| IL7 alpha | | |
| extracellular | | |
| domain | | |
| IL7 alpha | 61 | CCTATCCTGCTGACAATCAGCATCCTGAGCTTTTTCAGCGTGGCC |
| Transmembrane | | CTGCTGGTCATCCTGGCCTGTGTGCTGTGG |
| domain | | |
| IL7 alpha | 62 | AAGAAGCGGATCAAGCCCATCGTGTGGCCCAGCCTGCCTGACCA |
| intracellular | | CAAGAAAACCCTGGAACACCTGTGCAAGAAGCCCCGGAAGAACC |
| domain | | TGAACGTGTCCTTCAATCCCGAGAGCTTCCTGGACTGCCAGATCC |
| | | ACAGAGTGGACGACATCCAGGCCAGGGACGAAGTGGAAGGCTTC |
| | | CTCCAGGACACATTCCCTCAGCAGCTGGAAGAGAGCGAGAAGCA |
| | | GAGACTCGGCGGAGATGTGCAGAGCCCTAATTGCCCTAGCGAGG |
| | | ACGTGGTCATCACCCCTGAGAGCTTCGGCAGAGATAGCAGCCTG |
| | | ACATGTCTGGCCGGCAATGTGTCCGCCTGTGATGCCCCTATCCTG |
| | | AGCAGTAGCAGAAGCCTGGATTGCAGAGAGAGCGGCAAGAACG |
| | | GCCCTCACGTGTACCAGGATCTGCTGCTGTCTCTGGGCACCACCA |
| | | ATAGCACACTGCCTCCACCTTTCAGCCTGCAAAGCGGCATCCTGA |
| | | CACTGAACCCTGTGGCTCAGGGACAGCCAATCCTGACAAGCCTG |
| | | GGCAGCAATCAAGAAGAGGCCTACGTCACCATGAGCAGCTTCTA |
| | | CCAGAACCAG |
| Jun Leucine | 63 | GAGAAAGTCAAGACCCTGAAGGCCCAGAACAGCGAGCTGGCCTC |
| Zipper | | TACCGCCAACATGCTGAGAGAACAGGTGGCCCAACTG |
| Fos Leucine | 64 | GCCGAGACAGACCAGCTGGAAGATGAGAAGTCTGCCCTCCAGAC |
| Zipper | | CGAGATCGCCAACCTGCTGAAAGAGAAAGAGAAGCTG |
| Truncated | 65 | AACGGCATTTGGAGCGAGTGGAGCGAAGCCAGAAGCTGGGATAC |
| GMCSF2Rb | | CGAGAGCGTGCTGCCTATGTGG |
| extracellular | | |
| domain | | |
| GMCSF2Rb | 66 | GTGCTCGCCCTGATCGTGATCTTCCTGACAATCGCCGTGCTGCTG |
| Transmembrane | | GCCCTG |
| domain | | |
| GMCSF2Rb | 67 | AGATTCTGTGGCATCTACGGCTACAGACTGCGGCGGAAGTGGGA |
| intracellular | | AGAGAAGATCCCCAATCCTAGCAAGAGCCACCTGTTCCAGAATG |
| domain | | GCTCTGCCGAGCTTTGGCCTCCTGGCAGCATGAGCGCCTTTACAT |
| | | CTGGCAGCCCTCCACATCAAGGCCCCTGGGGAAGCAGATTCCCTG |
| | | AACTGGAAGGCGTGTTCCCTGTCGGCTTCGGCGATAGCGAAGTGT |
| | | CCCCTCTGACCATCGAGGACCCTAAGCACGTGTGCGATCCTCCAA |
| | | GCGGCCCTGATACAACACCAGCCGCAAGCGATCTGCCTACCGAG |
| | | CAACCTCCTAGTCCTCAGCCTGGACCTCCTGCCGCTTCTCACACA |
| | | CCTGAGAAACAGGCCAGCTCCTTCGACTTCAACGGCCCCTATCTG |
| | | GGCCCTCCTCATAGCAGAAGCCTGCCTGACATTCTGGGCCAGCCT |
| | | GAGCCTCCTCAAGAAGGCGGATCTCAGAAGTCTCCTCCACCTGGC |
| | | AGCCTGGAATACCTGTGTTTGCCTGCTGGTGGCCAGGTGCAGCTG |
| | | GTTCCTCTTGCTCAAGCTATGGGACCTGGACAGGCCGTGGAAGTG |
| | | GAAAGAAGGCCATCTCAAGGCGCCGCTGGCTCTCCATCTCTTGAA |
| | | TCTGGCGGAGGACCTGCTCCTCCTGCTCTGGGACCTAGAGTTGGA |
| | | GGCCAGGACCAGAAAGACAGCCCCGTGGCCATTCCTATGAGCAG |
| | | CGGAGATACCGAAGATCCCGGCGTGGCCTCTGGCTATGTGTCATC |
| | | TGCCGACCTGGTGTTCACCCCTAATAGCGGCGCTAGCTCCGTGTC |
| | | TCTGGTGCCTAGTCTTGGCCTGCCTAGCGATCAGACCCCTTCTCTG |
| | | TGTCCTGGACTGGCTTCTGGACCACCTGGTGCTCCAGGACCTGTG |
| | | AAGTCTGGCTTTGAGGGCTACGTGGAACTGCCTCCTATCGAGGGC |
| | | AGAAGCCCTAGAAGCCCCAGAAACAACCCTGTGCCTCCTGAGGC |
| | | CAAGTCTCCCGTTCTGAACCCTGGCGAAAGACCCGCCGATGTGTC |
| | | TCCTACATCTCCTCAGCCAGAGGGACTGCTGGTGCTGCAACAAGT |
| | | GGGCGACTACTGCTTCCTGCCTGGACTTGGACCAGGACCTCTGAG |
| | | CCTGAGAAGCAAGCCTAGCTCTCCTGGACCTGGGCCTGAGATCAA |
| | | GAATCTGGACCAGGCCTTCCAAGTGAAGAAGCCTCCAGGCCAGG |
| | | CTGTGCCTCAGGTGCCAGTGATCCAGCTGTTCAAGGCCCTGAAGC |
| | | AGCAGGACTACCTGAGCCTGCCACCTTGGGAAGTGAACAAGCCA |
| | | GGCGAAGTGTGC |
| Truncated | 68 | AACTGGTCCTCTTGGAGCGAGGCCATCGAGTTCGGCAGCGACGAT |
| GMCSF2Ra | | GGC |
| extracellular | | |
| domain | | |
| GMCSF2Ra | 69 | AATCTGGGCAGCGTGTACATCTACGTGCTGCTGATCGTGGGCACC |
| Transmembrane | | CTCGTGTGTGGAATCGTGCTGGGCTTCCTGTTC |
| domain | | |
| GMCSF2Ra | 70 | AAGCGGTTCCTGAGAATCCAGCGGCTGTTCCCTCCAGTGCCTCAG |
| Intracellular | | ATCAAGGACAAGCTGAACGACAACCACGAGGTCGAGGACGAGAT |
| domain | | CATCTGGGAAGAGTTCACCCCTGAGGAAGGCAAGGGCTACAGAG |
| | | AAGAGGTGCTGACCGTGAAAGAGATCACC |
| Truncated | 71 | AGCCCTCTGTTTCCCGGACCTAGCAAG |
| CD28 | | |
| extracellular | | |
| domain | | |
| CD28 | 72 | CCTTTCTGGGTGCTCGTTGTTGTTGGCGGCGTGCTGGCCTGTTACA |
| Transmembrane | | GCCTGCTGGTTACCGTGGCCTTCATCATCTTTTGGGTC |
| domain | | |
| Truncated | 73 | TCCCCGCTCTTCCCGGGCCCCTCCAAACCG |
| CD28 | | |
| extracellular | | |
| domain | | |
| (Wobbled) | | |
| CD28 | 74 | TTTTGGGTCCTGGTGGTAGTAGGTGGTGTCTTGGCgTGcTAttccCTc |
| Transmembrane | | CTcGTgACgGTcGCgTTtATtATtTTcTGGGTg |
| domain | | |
| (Wobbled) | | |
| Anti- | 75 | GACATCCAGATGACACAGAGCCCTAGCAGCCTGTCTGCCAGCGT |
| BHA10 | | GGGAGACAGAGTGACCATCACATGCAAGGCCAGCCAGAACGTGG |
| Light chain | | GCATCAACGTGGCCTGGTATCAGCAGAAGCCTGGCAAGGCCCCT |
| | | AAGAGCCTGATCAGCAGCGCCAGCTACAGATACAGCGGCGTGCC |
| | | AAGCAGATTTTCCGGCTCTGGCAGCGGCACCGATTTCACCCTGAC |
| | | AATATCCAGCCTCCAGCCTGAGGACTTCGCCACCTACTTTTGCCA |
| | | GCAGTACGACACATACCCCTTCACCTTCGGCTGCGGCACCAAGGT |
| | | GGAAATCAAG |
| Anti- | 76 | CAGGTTCAGCTGGTTCAGTCTGGCGCCGAGGAAGTGAAGAAACC |
| BHA10 | | TGGCAGCTCTGTGAAGGTGTCCTGCAAGGCCAGCGGCTACACCTT |
| Heavy chain | | TACCACCTACTACCTGCACTGGGTCCGACAGGCTCCAGGCCAATG |
| | | TCTGGAATGGATGGGCTGGATCTACCCCGGCAATGTGCACGCCCA |
| | | GTACAACGAGAAGTTCAAGGGCAGAGTGACCATCACCGCCGACA |
| | | AGAGCACAAGCACCGCCTACATGGAACTGAGCAGCCTGAGAAGC |
| | | GAGGACACCGCCGTGTACTACTGTGCCAGAAGCTGGGAGGGCTT |
| | | CCCTTATTGGGGCCAGGGAACCACCGTGACCGTTAGCTCT |
| FAB Light | 77 | AGAACAGTGGCCGCTCCTAGCGTGTTCATCTTCCCACCTTCCGAC |
| chain | | GAGCAGCTGAAGTCTGGCACAGCCTCTGTCGTGTGCCTGCTGAAC |
| | | AACTTCTACCCCAGAGAAGCCAAGGTGCAGTGGAAGGTGGACAA |
| | | CGCCCTCCAGAGCGGCAATAGCCAAGAGAGCGTGACCGAGCAGG |
| | | ACAGCAAGGACTCTACCTACAGCCTGAGCAGCACCCTGACACTG |
| | | AGCAAGGCCGACTACGAGAAGCACAAAGTGTACGCCTGCGAAGT |
| | | GACCCACCAGGGCCTTTCTAGCCCTGTGACCAAGAGCTTCAACCG |
| | | GGGAGAGTGCGAGCCCAAGAGCTGCGATAAGACCCACACCTGTC |
| | | CTCCATGTCCT |
| FAB Heavy | 78 | GCCTCTACAAAGGGCCCTAGTGTGTTCCCTCTGGCTCCCAGCAGC |
| chain | | AAGTCTACATCTGGCGGAACAGCCGCTCTGGGCTGCCTGGTCAAG |
| | | GATTACTTTCCCGAGCCTGTGACCGTGTCCTGGAATAGCGGAGCA |
| | | CTGACAAGCGGCGTGCACACCTTTCCAGCTGTGCTGCAAAGCAGC |
| | | GGCCTGTACTCTCTGAGCAGCGTGGTCACAGTGCCTAGCTCTAGC |
| | | CTGGGCACCCAGACCTACATCTGCAATGTGAACCACAAGCCTAGC |
| | | AACACCAAGGTGGACAAGAAGGTGGAACCCAAGAGCTGCGACA |
| | | AGACCCACACCTGTCCTCCATGTCCT |

Recombinant proteins were produced in CHO cells. The heavy and light chains gene of the antibodies or antibody fragments were cloned into the pCDNA expression vector containing a strong promoter. The vector was transfected into CHO cells, and supernatant harvested after 5 days. Protein was purified through either a protein A or His-tag affinity chromatography column. FAb fragments were generated via pepsin (Thermo Fisher) cleavage.

After generating the antibody fragments for the constructs of SEQ ID NOS: 25, 31, 54, and 55, the proteins were purified and ran on SDS-PAGE gels (FIGS. 2B-2D). Proteins were column purified based on His tag presence using immobilised metal ion affinity chromatography. Purification and molecular weight were confirmed via SDS-PAGE which highlighted the presence of protein at the expected molecular weights (FAB-40 kDa, dsFv-20 kDa, Fv-10 kDa). Specificity of protein and stability of dsFv were established using an enzyme linked immunosorbent assay (ELISA) whereby plates were coated with immobilized TBRII receptor and treated with proteins at different concentrations. Binding was detected based on detection of the His tag in the protein and showed comparable binding with FAB and dsFv proteins but lack of signal with Fv.

Next, ELISAs were run on the antibody fragments against immobilized TBRII receptor (FIGS. 2E-2G). Plates were coated with immobilised TBRII receptor ligand (R&D Systems, 341-BR-050) overnight at 4° C. and blocked using assay buffer for an hour. All wash steps were done using PBS+0.5% Tween (Sigma Aldrich). Proteins were serially diluted and added to the plate and allowed to bind for 2 hours. After washing, the plate was treated with a detection antibody and an anti-his HRP (GenScript). Development was using TMB substrate and stop solution (BioLegend, 421101, 423001). Readout was taken at 450 nm using a plate reader (Vario scanner, Thermofisher). The FAB portion was calculated to have an EC₅₀ of 0.002753 μM, the FV portion was calculated to have an EC₅₀ of 0.007776 μM, and the dsFv portion was calculated to have an EC₅₀ of 0.002212 μM.

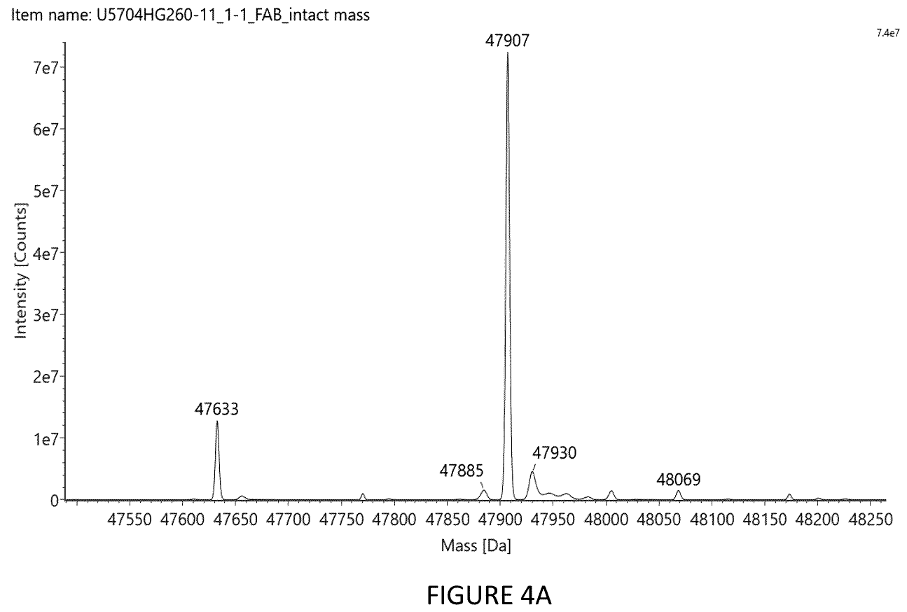

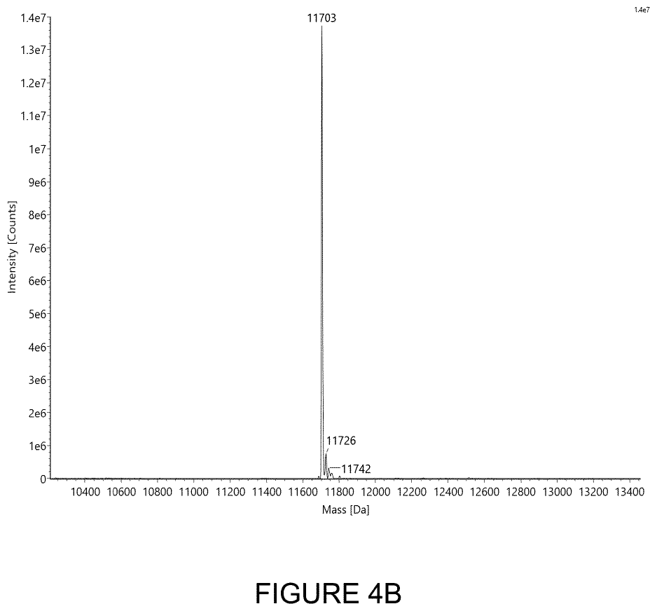

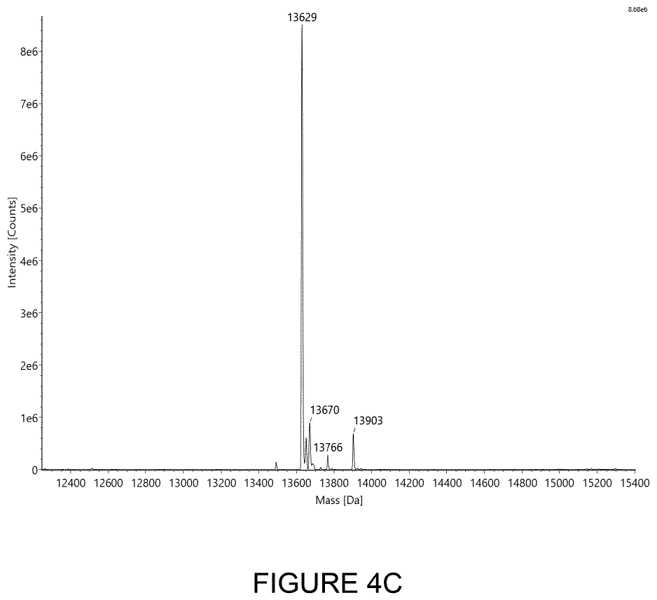

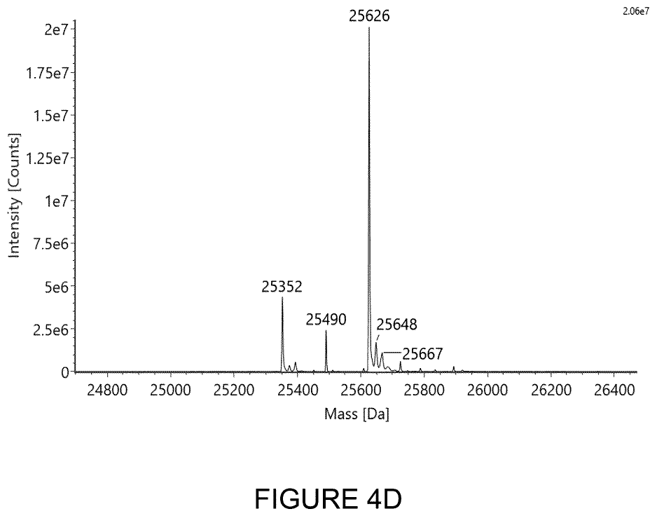

The antibody fragments were then subjected to deconvoluted intact mass spectra (FIGS. 4A-4D). Purified protein samples were prepared by dialyzing and concentrating it in a volatile buffer. Mass spectra were obtained using an Agilent mass spectrometer in positive ion mode with a resolution of at least 10,000. Deconvolution of the mass spectra was performed using MassHunter software to obtain the intact mass of the protein. Data analysis was conducted by comparing the deconvoluted mass to the expected mass based on the amino acid sequence and identifying any impurities or variants using BioPharmaFinder software. The experimental derived masses of major glycoform containing species agreed well with the theoretical masses of the proteins indicating that the disulfide bond for the dsFv format antibody remained intact (FIG. 4D). The Fv produced proteins demonstrated two distinct masses corresponding to the heavy and light variable regions of the protein (FIGS. 4B-4C).

### Example 2: DsFv Protein Targeting TBRII Inhibits TGF-β Signaling

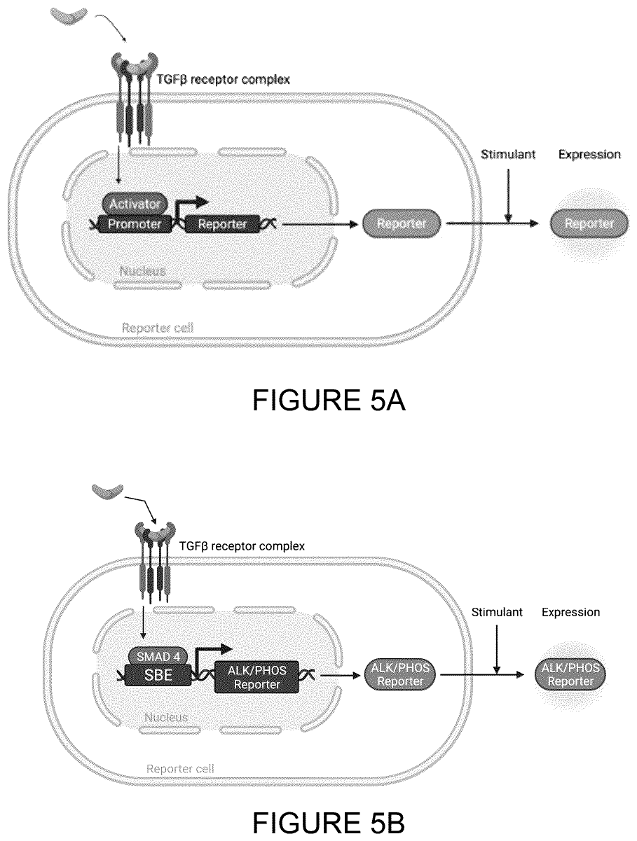

The function of anti-TBRII blocking FAB (SEQ ID NOs: 81-82) and/or a dsFv (including SEQ ID NO: 25) was determined using a reporter cell line in which TGFBRI and TGFBRII are expressed on the cell surface (FIGS. 5A-5B). Upon ligation of TGF-β, downstream signaling results in the transcription and production of an alkaline phosphatase that is secreted into the culture supernatant. Upon treatment with a dephosphorylating agent, a chemiluminescent signal is produced and detected.

Reporter cell lines were maintained in Dulbecco's Modified Eagle Medium (DMEM) media (Sigma Aldrich) supplemented with 10% FBS (Biosera, FB-1001) and 2 mM GlutaMax (Gibco) (complete media). 2× 10⁴ cells were seeded into wells of a flat bottom 96 well plate (Corning) in the presence of recombinant human TGF-β (R&D Systems, 7754-BH-005/CF) at varying concentrations and/or proteins specifically targeting TBRII. Recombinant TGF-β was acid treated for activation using hydrochloric acid (RB04). Plates were maintained in 37° C. and 5% CO₂ for 24 hours, upon which supernatant was harvested and treated with a dephosphorylating substrate (InVivoGen, rep-qbs3) added and read out taken after 1 hour. Measurement was taken at the 630 nm wavelength using a plate reader (Vario Scanner, Thermofisher).

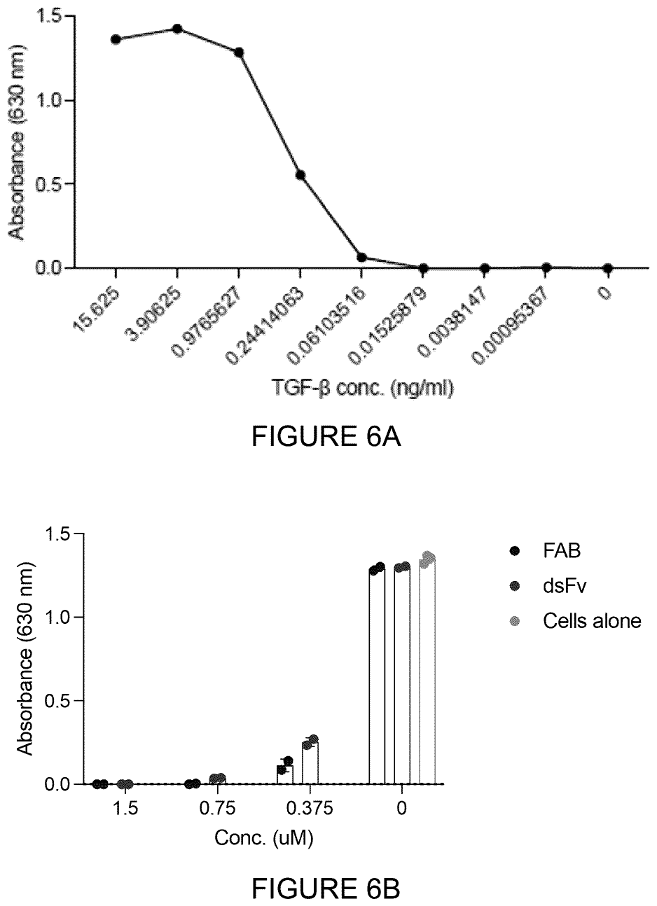

A range of concentrations of recombinant TGF-β cytokine was pulsed over the reporter cell line for 48 hrs and signal detected (FIG. 6A). From this assay, 1 ng/ml of TGF-β was used for subsequent testing. As shown in FIG. 6A, the cell line was responsive to TGF-β.

FAB and dsFV proteins specific for TBRII were cultured with the reporter cell line at concentrations ranging from 1.5 μM to 0 μM in the presence of TGF-β (FIG. 6B). As a control, the reporter cell line alone was cultured with TGF-β in the absence of proteins. The blocking capacity of the dvFv protein was comparable to the FAB protein at the higher concentrations, demonstrating the ability of the anti-TBRII dsFv to inhibit TGF-β signaling.

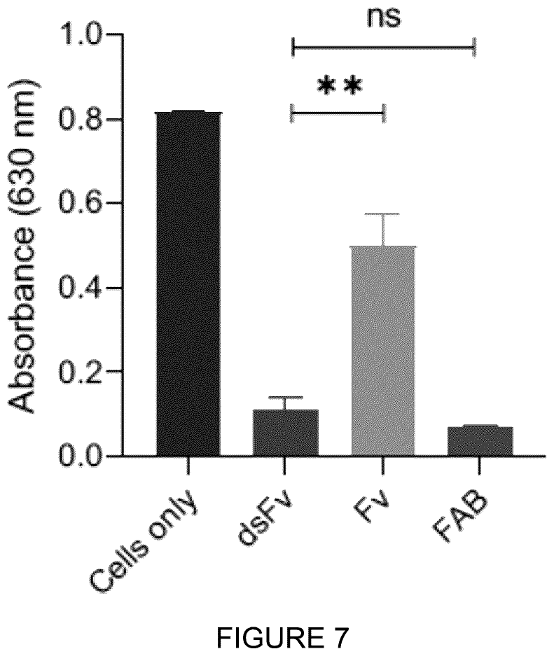

In a subsequent experiment, cells with a set concentration of dsFv (including SEQ ID NO: 25), wild type Fv, or wild type FAB and TGF-β were monitored for the release of alkaline phosphatase (FIG. 7). Reporter cell lines were maintained in Dulbecco's Modified Eagle Medium (DMEM) media (Sigma Aldrich) supplemented with 10% FBS (Biosera, FB-1001) and 2 mM GlutaMax (Gibco) (complete media). 2× 10⁴ cells were seeded into wells of a flat bottom 96 well plate (Corning, 3595) in the presence of recombinant human and/or proteins specifically targeting TBRII at varying concentrations. Plates were maintained in 37° C. and 5% CO₂ for 24 hours, upon which supernatant was harvested and treated with a dephosphorylating substrate (InVivoGen, rep-qbs3) added and read out taken after 1 hour. Measurement was taken at the 630 nm wavelength using a plate reader (Vario Scanner, Thermofisher). When used in the context of a TBRII blocking antibody in a TGF-β sensitive reporter cell assay, the presence of the disulfide bond between the variable heavy and light chain enhances the blocking function of the dsFV compared to the FV. The improvement showed the dsFv provided similar functionality to a TBRII targeting FAB protein.

### Example 3: Dual Blocking and Constitutive Cytokine Signaling Module Blocks TGF-β Signaling

As discussed in the above Example 2 and depicted in FIGS. 5A-5B, one experiment assay includes a reporter cell line expressing TBRI and TBRII which is ligated by TGF-β, resulting in the production of an alkaline phosphatase that is secreted into the culture supernatant. Upon treatment with a dephosphorylating agent, a chemiluminescent signal is produced and detected.

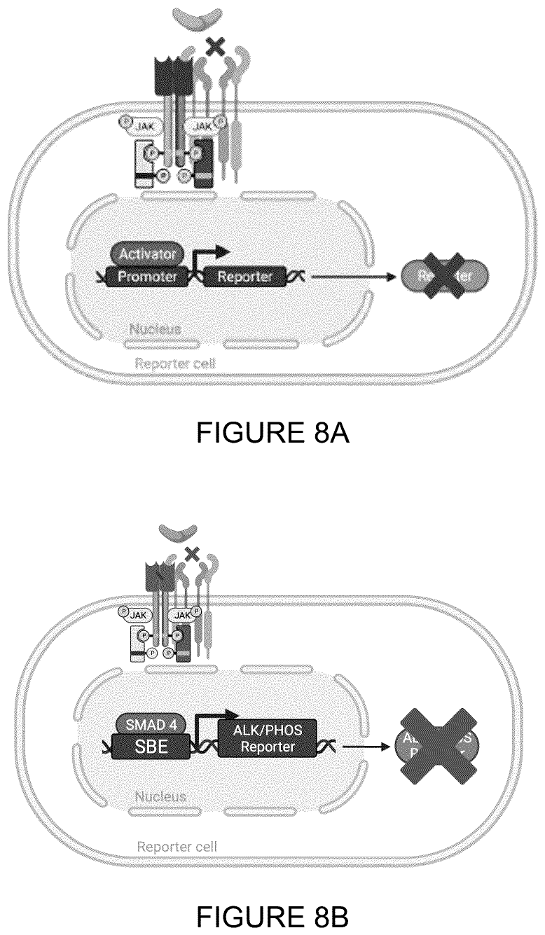

For the next step, the previously generated virus was used to infect the TGF-β reporter cell line to co-express the anti-TBRII disulfide bond stabilized Fv (including SEQ ID NO: 25) on the cell surface linked to the signaling chains of a cytokine receptor. This was expected to prevent TGF-β from engaging with TBRI and TBRII receptor complex to block the alkaline phosphatase reporter generation and secretion (FIGS. 8A-8B).

Reporter cell lines were maintained in Dulbecco's Modified Eagle Medium (DMEM) media (Sigma Aldrich) supplemented with 10% FBS (Biosera, FB-1001) and 2 mM GlutaMax (Gibco) (complete media). γ-retroviruses produced following the transfection of HEK 293T cells, viruses were harvested and used to transduce cell lines in the presence of polybrene (Sigma Aldrich, TR-1003-G). Cell lines were treated with virus for three consecutive days and transgene expression measured via flow cytometry (Miltenyi Biotech, MacsQuant 10) at day four.

2×10⁴ cells were seeded into wells of a flat bottom 96 well plate (Corning, 3595) in the presence of recombinant human TGF-β (R&D Systems, 7754-BH-005/CF) at varying concentrations and/or proteins specifically targeting TBRII. Recombinant TGF-β was acid treated for activation using hydrochloric acid (RB04). Plates were maintained in 37° C. and 5% CO₂ for 24 hours, upon which supernatant was harvested and treated with a dephosphorylating substrate (InVivoGen, rep-qbs3) added and read out taken after 1 hour. Measurement was taken at the 630 nm wavelength using a plate reader (Vario Scanner, Thermofisher).

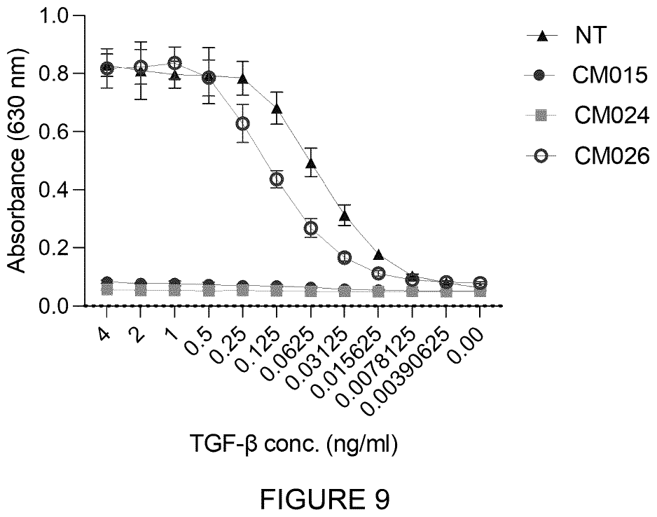

TGF-β reporter cells alone, or the cells expressing one of either CM015, CM024, or CM026 construct variants co-cultured in the presence of a range of concentrations of recombinant TGF-β for 48 hours and alkaline phosphatase presence quantified. Blocking was established as a decrease or absence in absorbance readout and was only seen in variants expressing aTBRII dsFv (TGFb block) (FIG. 9). When a non-specific dsFv was expressed on the surface of the TGF-β reporter cell line, the absorbance values generated were equivalent to the unmodified TGF-reporter cell line (NT).

### Example 4: Constitutive Cytokine Chains Provide Greater Signaling than Recombinant Cytokines

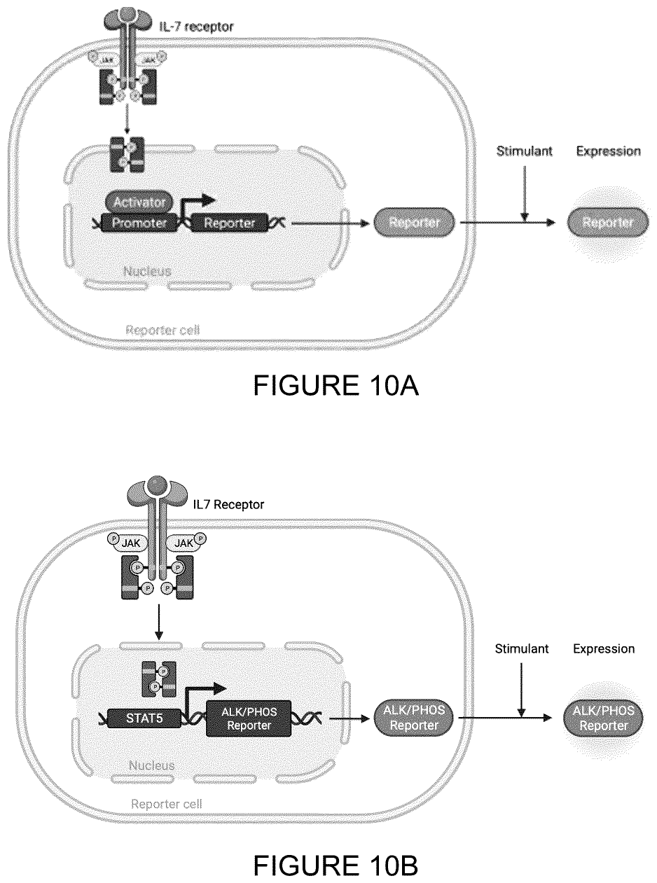

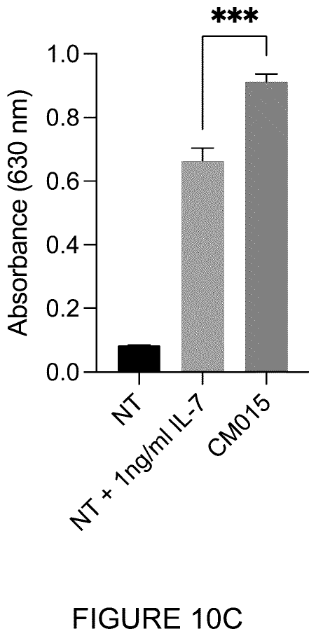

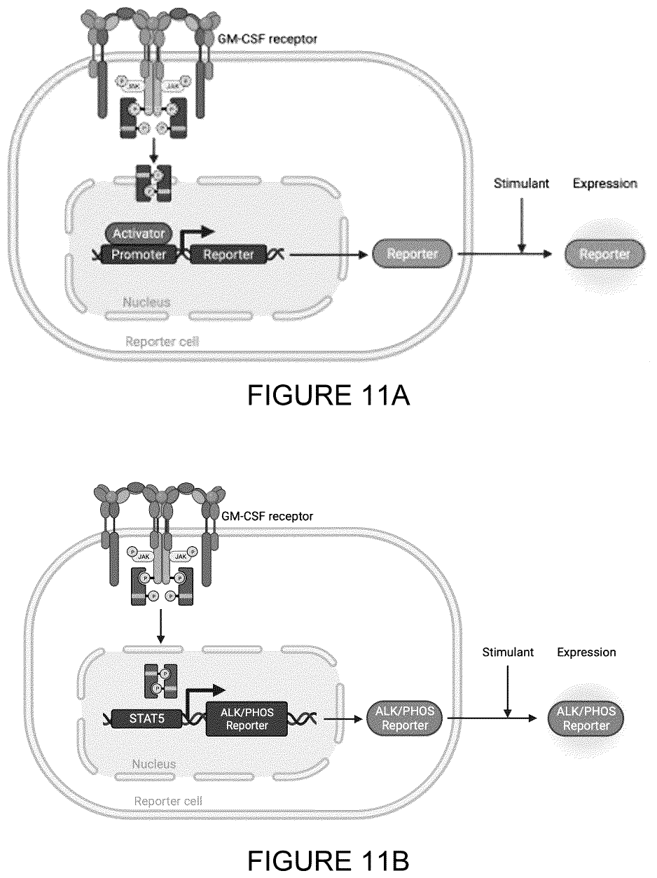

As a further derivation of the cell-based reporter assay as shown in FIGS. 5A-5B, the inventors also constructed a reporter cell line in which the IL-7 receptor is expressed on the cell surface (FIGS. 10A-10B). Upon ligation by IL-7, downstream JAK/STAT signaling results in the transcription and production of an alkaline phosphatase that is secreted into the culture supernatant. Upon treatment with a dephosphorylating agent, a chemiluminescent signal is produced and detected to quantify activity.

A variant of the reporter cell line was also generated by the inventors, in which the cell expressed the anti-TBRII dsFv (construct CM015, including SEQ ID NO: 25) with constitutive IL-7 signaling. Reporter cell lines were maintained in Dulbecco's Modified Eagle Medium (DMEM) media (Sigma Aldrich) supplemented with 10% FBS (Biosera, FB-1001) and 2 mM GlutaMax (Gibco) (complete media). γ-retroviruses produced following the transfection of HEK 293T cells, viruses were harvested and used to transduce cell lines in the presence of polybrene (Sigma Aldrich, TR-1003-G). Cell lines were treated with virus for three consecutive days and transgene expression measured via flow cytometry (Miltenyi Biotech, MacsQuant 10) at day four.

2×10⁴ cells were seeded into wells of a flat bottom 96 well plate in the presence or absence of recombinant human IL-7 (Peprotech, 200-07) or GM-CSF (Peprotech, 300-23) at 1 ng/ml. Plates were maintained in 37° C. and 5% CO₂ for 24 hours, upon which supernatant was harvested and treated with a dephosphorylating substrate (InVivoGen, rep-qbs3) added and read out taken after 1 hour. Measurement was taken at the 630 nm wavelength using a plate reader (Vario Scanner, Thermofisher).

The signal generated by this variant was determined upon 48 hours culture and quantified against reporter cell lines co-cultured with 1 ng/ml of recombinant IL-7. The signal generated significantly exceeded the signal seen in the presence of recombinant cytokine (FIG. 10C).

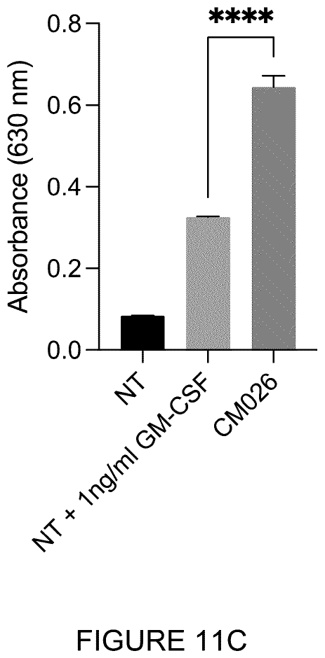

A fourth reporter cell line assay was also created, in which the GM-CSF receptor is expressed on the cell surface (FIGS. 11A-11B). Upon ligation by GM-CSF, downstream JAK/STAT signaling results in the transcription and production of an alkaline phosphatase that is secreted into the culture supernatant. Upon treatment with a dephosphorylating agent, a chemiluminescent signal is produced and detected to quantify activity.

This variant of the reporter cell line was generated by the inventors, in which the cell expressed the anti-TBRII dsFv with constitutive GMCSF signaling (construct CM026). The signal generated by this variant was determined upon 48 hours culture and quantified against reporter cell lines co-cultured with 1 ng/ml of recombinant GM-CSF (FIG. 11C).

### Example 5: Constitutive Cytokine Signaling Enhances Cell Proliferation

Peripheral blood mononuclear cells (PBMCs) are isolated from healthy donor blood samples and cultured in the presence of anti-CD3 and anti-CD28 antibodies with recombinant IL-2 for 48 hours. Post activation, cells are transduced with virus encoding a construct consisting of anti-CD19 CAR only or anti-CD19 CAR and anti-TBRII dsFv with constitutive cytokine receptor or an anti-CD19 CAR and anti-TBRII dsFv with truncated cytokine receptor.

Incorporation of the virus into the cells is determined by transgene detection using flow cytometry. Co-cultures are set up against target cells expressing CD19 or lacking CD19 expression. Cytotoxicity is determined as a ratio of T cells to target cells present within the co-culture and determined every 3 days. Upon readout, fresh target cells are seeded onto remaining co-culture plates. In cultures with CAR only or CAR and anti-TBRII with truncated cytokine receptor, after 3-4 rounds of antigen challenge, the T cell proportion decreases, and cells recovered from these wells are target cells. In comparison, by 9-10 rounds of re-stimulation in co-cultures with anti-CD19 CAR and anti-TBRII dsFv with constitutive cytokine receptor, target cells are not detectable with only T cells recovered in these wells. This demonstrates that the incorporation of a constitutive cytokine receptor vastly improves the long-term cytotoxic function of these cells when repeatedly challenged with antigen.

Cultures are established wherein cells are seeded in culture media in the absence of additional simulation or exogenous cytokines. Cells are harvested and counted every 3 days and re-seeded until no longer viable. Significantly greater cell counts are seen when cells expressing a constitutive cytokine receptor are cultured, whilst non-transduced or cells expressing the truncated cytokine receptor are no longer viable by day 6 of culture.

### Example 6: TGF-β Inhibition by Anti-TBRII dsFv Expressing CAR T-Cells

Peripheral blood mononuclear cells (PBMCs) are isolated from health donor blood samples and cultured in the presence of anti-CD3 and anti-CD28 antibodies with recombinant IL-2 for 48 hours. Post activation, cells are transduced with virus encoding a construct consisting of: (1) anti-CD19 CAR only, (2) anti-CD19 CAR and anti-TBRII dsFv with constitutive cytokine receptor, (3) an anti-CD19 CAR and a nonspecific dsFv with constitutive cytokine receptor, or (4) an anti-CD19 CAR and an anti-TBRII dsFv with truncated cytokine receptor.

Incorporation of the viruses is determined by transgene detection using flow cytometry. Co-cultures are then set up against target cells expressing CD19 or wild-type variants in the presence of absence of recombinant TGF-β cytokine. Cultures are maintained for 1 week upon which cytotoxicity was measured via flow cytometry.

Proportion of cytotoxicity is determined as a percentage of cells recovered compared to non-transduced T cells cultured with antigen negative target cells. In the presence of TGF-β, cytotoxicity of anti-CD19 CAR is inhibited with comparable levels of target cells recovered compared to controls. In comparison, the greatest degree of cytotoxicity in the presence of TGF-β is seen in cultures with CARs co-expressing the anti-TBRII dsFv.

IFN-gamma from these co-cultures is measured via an ELISA, correlating with target cell survival. Decreased IFN-gamma is seen in the presence of TGF-β when the anti-TBRII dsFv is not expressed.

### Example 7: Long Term Cytotoxicity Upon Continuous Antigen Exposure

Peripheral blood mononuclear cells (PBMCs) are isolated from health donor blood samples and cultured in the presence of anti-CD3 and anti-CD28 antibodies with recombinant IL-2 for 48 hours. Post activation, cells are transduced with virus encoding a construct consisting of: (1) anti-CD19 CAR only, (2) anti-CD19 CAR and anti-TBRII dsFv with constitutive cytokine receptor, (3) or an anti-CD19 CAR and anti-TBRII dsFv with truncated cytokine receptor.

Incorporation of the viruses is determined by transgene detection using flow cytometry. Co-cultures are set up against target cells expressing CD19 or lacking CD19 expression. Cytotoxicity is determined as a ratio of T cells to target cells present within the co-culture and determined every 3 days.

Upon readout, fresh target cells are seeded onto remaining co-culture plates. In cultures with CAR only or CAR and anti-TBRII with truncated cytokine receptor, after 3-4 rounds of antigen challenge, the T cell proportion decreases and cells recovered from these wells are target cells. In comparison, by 9-10 rounds of re-stimulation in co-cultures with anti-CD19 CAR and anti-TBRII dsFv with constitutive cytokine receptor, target cells are not detectable with only T cells recovered in these wells. This demonstrates that the incorporation of a constitutive cytokine receptor vastly improves the long-term cytotoxic function of these cells when repeatedly challenged with antigen.

### Example 8: Orientation of Cytokine Receptor Chains Regulates IL-7 Constitutive Signaling

The anti-TBRII dsFv/GM-CSF CCR construct is tested with the cytokine receptor chain in the αβ and βα orientations (SEQ ID NOs: 27-35), to screen for the differences in signal generation.

A reporter cell line is generated, in which the IL-7 receptor is expressed on the cell surface. Upon ligation by IL-7, downstream JAK/STAT signaling results in the transcription and production of an alkaline phosphatase that is secreted into the culture supernatant. Upon treatment with a dephosphorylating agent, a chemiluminescent signal is produced and detected to quantify activity.

Variants of the receptor cell line are generated that expressed the anti-TBRII dsFv (SEQ ID NO: 25) with constitutive IL-7 signaling in either orientation. The signal generated by each cell is determined after 48 hours of culturing, and quantified against reporter cell lines co-cultured with 1 ng/ml of recombinant IL-7. The differences in signal is observed. The signal can be modified by altering the orientation of the IL7 cytokine receptor chains, with the αγ orientation showing decreased IL-7 signaling compared to the γα variant.

Peripheral blood mononuclear cells (PBMCs) are isolated from health donor blood samples and cultured in the presence of anti-CD3 and anti-CD28 antibodies with recombinant IL-2 for 48 hours. Post activation, cells are transduced with the constructs. Cultures are established wherein cells are seeded in culture media in the absence of additional simulation or exogenous cytokines. Cells are harvested after 6 days and cells counted, survival and growth are assessed compared to the number of cells recovered from the non-transduced (NT) population and fold change calculated. A similar trend is observed whereby the ya orientation provides greater cell survival and expansion compared to the IL7 receptor ay orientation.

### Example 9: Enhanced Cytokine Signaling with Dimerization Motifs

To achieve maximal signalling from the anti-TBRII/CCR, constructs are modified to include additional motifs that promote dimerization and aggregation of cytokine receptors to maximise signalling. In particular, the inclusion of leucine zippers are examined for their effect to maximize cytokine signalling.

Variants of the reporter cell line are generated that express the anti-TBRII dsFv (SEQ ID NO: 25) with constitutive IL-7 signalling with and without additional motifs. The signal generated by this variant is determined upon 48 hours culture and quantified against reporter cell lines co-cultured with 1 ng/ml of recombinant IL-7. The signal generated significantly exceeds the signal seen in the presence of recombinant cytokine.

A reporter cell line is generated in which the GM-CSF receptor is expressed on the cell surface. Upon ligation by GM-CSF, downstream JAK/STAT signalling results in the transcription and production of an alkaline phosphatase that is secreted into the culture supernatant. Upon treatment with a dephosphorylating agent, a chemiluminescent signal is produced and detected to quantify activity.

A variant of the reporter cell line is generated that expresses the anti-TBRII dsFv with constitutive GM-CSF signalling with and without dimerization motifs. The signal generated by this variant is determined upon 48 hours culture and quantified against reporter cell lines co-cultured with 1 ng/ml of recombinant GM-CSF.

### Example 10: Constitutive Cytokine Chains Provide a Growth Advantage

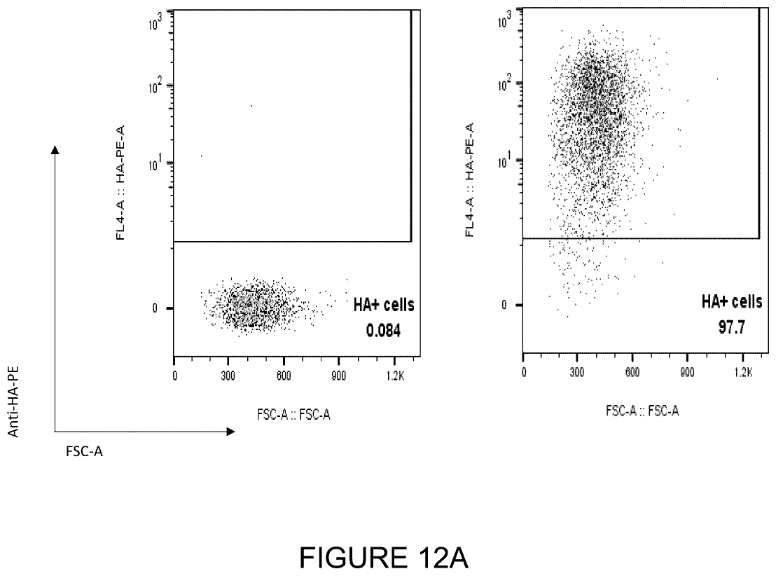

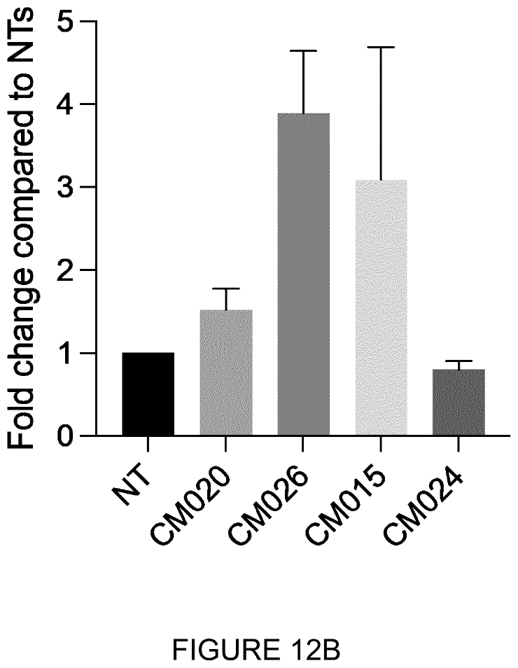

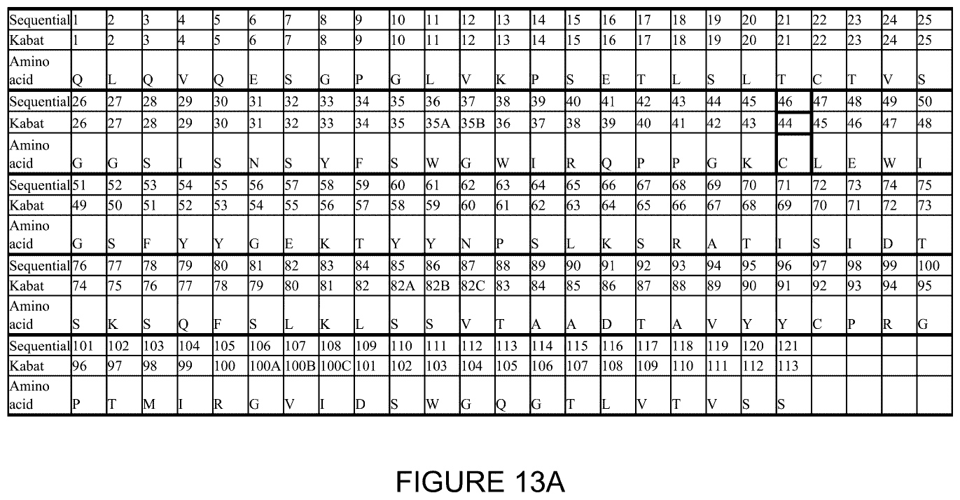

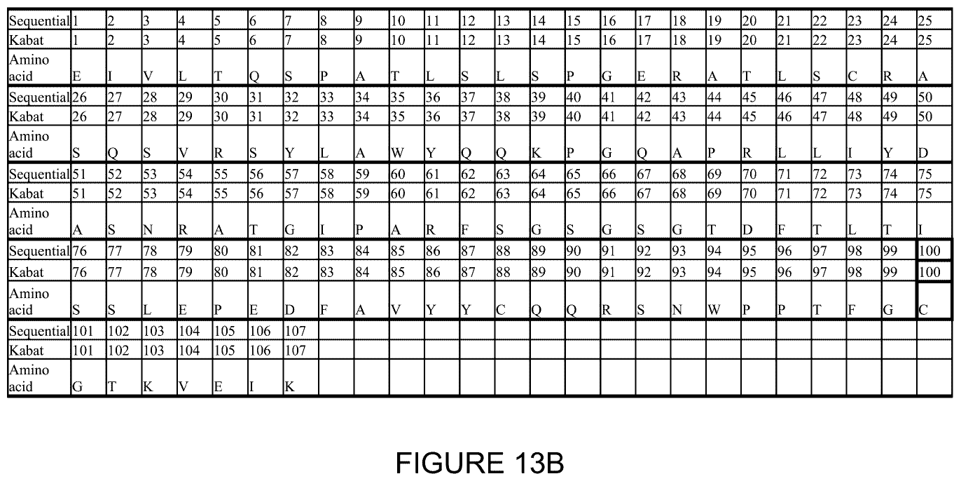

Peripheral blood mononuclear cells (PBMCs) were isolated from healthy donor blood samples and cultured in the presence of anti-CD3 and anti-CD28 antibodies with recombinant IL-2 for 48 hours. Post activation, cells were transduced with virus encoding the anti-TBRII dsFv (SEQ ID NO: 25) and a variant of a constitutive cytokine receptor or an anti-TBRII dsFv with a truncated cytokine receptor. Incorporation of virus was determined by transgene detection using flow cytometry (FIG. 12A, left and right panels). Cultures were established wherein cells were seeded in culture media in the absence of additional simulation or exogenous cytokines. Cells were harvested after 6 days and cells counted, survival and growth were assessed compared to the number of cells recovered from the non-transduced (NT) population and fold change were calculated (FIG. 12B).

PBMCs were isolated from buffy cones from healthy donors (National health service, blood, and transplant) via density centrifugation. Isolated PBMCs were stimulated with anti-CD3 (Miltenyi Biotech, 130-093-387) and anti-CD28 (Miltenyi Biotech, 130-093-375)+recombinant human IL-2 (Miltenyi Biotech, 130-097-746) for 48 hours. Cells were transduced with γ-retroviruses produced following the transfection of HEK 293T cells. Cells and viruses were added to retronectin (Takara, T100B coated plates and spinoculated (Thermo Fisher, 17171925). Cultures were maintained for 48 hours at 37° C. and 5% CO₂ prior to harvesting with transduction efficiency measured via flow cytometry (Miltenyi Biotech, MacsQuant 10).

2.5×10⁵ transduced cells were seeded into a 24 well plate and maintained for 6 days at 37° C. and 5% CO₂. Cell were harvested and counted (Thermo fisher, Countess III).

### Example 11: TGF-β Inhibition by Anti-TBRII dsFv Expressing CAR T Cells

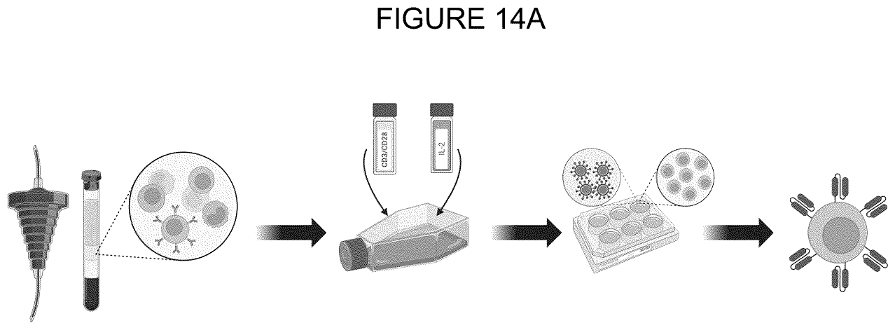

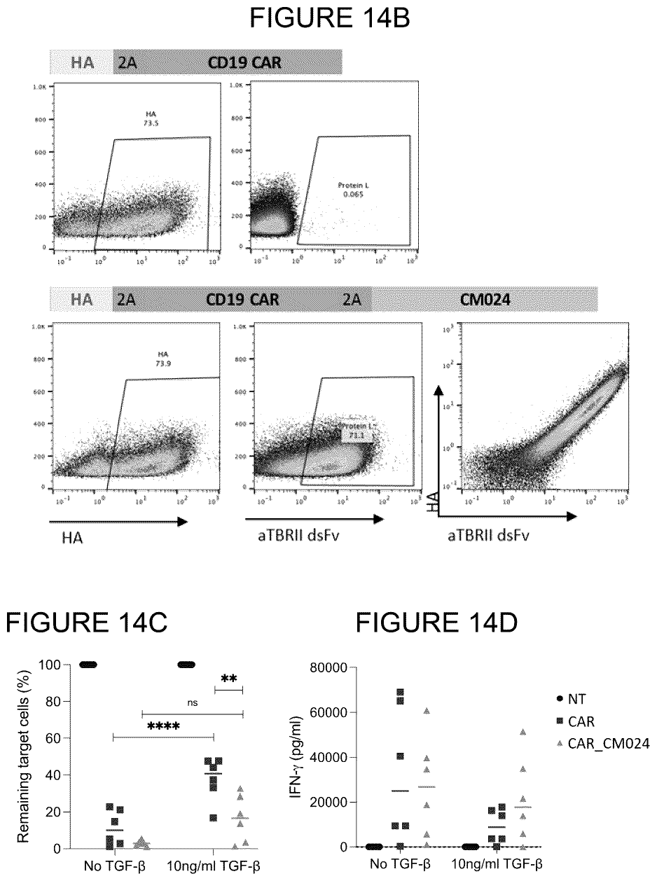

Peripheral blood mononuclear cells (PBMCs) were isolated from healthy donor blood samples and cultured in the presence of anti-CD3 and anti-CD28 antibodies with recombinant IL-2 for 48 hours. Post activation, cells were transduced with virus encoding a construct consisting of anti-CD19 CAR only or anti-CD19 CAR and anti-TBRII dsFv with constitutive cytokine receptor or an anti-CD19 CAR and a non-specific dsFv with constitutive cytokine receptor or an anti-CD19 CAR and an anti-TBRII dsFv with truncated cytokine receptor (FIG. 14A). Incorporation of virus was determined by transgene detection using flow cytometry (FIG. 14B).

Co-cultures were set up against target cells expressing CD19 or wild-type variants in the presence of absence of recombinant TGF-β cytokine. Cultures were maintained for 1 week upon which cytotoxicity was measured via flow cytometry. The proportion of cytotoxicity was determined as a percentage of cells recovered compared to non-transduced T cells cultured with antigen negative target cells. In the presence of TGF-β cytotoxicity of anti-CD19, CAR only was inhibited with comparable levels of target cells recovered compared to controls (FIG. 14AC). In comparison, the greatest degree of cytotoxicity in the presence of TGF-β was seen in cultures with CARs co-expressing the anti-TBRII dsFv. IFN-γ from these co-cultures was measured via an ELISA, correlating with target cell survival, decreased IFN-γ was seen in the presence of TGF-β when the anti-TBRII dsFv was not expressed (FIG. 14D).

### Example 12: Long-Term Cytotoxicity Upon Continuous Antigen Exposure

Peripheral blood mononuclear cells (PBMCs) are isolated from healthy donor blood samples and cultured in the presence of anti-CD3 and anti-CD28 antibodies with recombinant IL-2 for 48 hours. Post activation, cells are transduced with virus encoding a construct consisting of anti-CD19 CAR only or anti-CD19 CAR and anti-TBRII dsFv with constitutive cytokine receptor or an anti-CD19 CAR and anti-TBRII dsFv with truncated cytokine receptor.

Incorporation of virus is determined by transgene detection using flow cytometry. Co-cultures are set up against target cells expressing CD19 or lacking CD19 expression. Cytotoxicity is determined as a ratio of T cells to target cells present within the co-culture and determined every 3 days. Upon readout, fresh target cells are seeded onto remaining co-culture plates. In cultures with CAR only or CAR and anti-TBRII with truncated cytokine receptor, after 3-4 rounds of antigen challenge, the T cell proportion decreases, and cells recovered from these wells are target cells. In comparison, by 9-10 rounds of re-stimulation in co-cultures with anti-CD19 CAR and anti-TBRII dsFv with constitutive cytokine receptor, target cells are not detectable with only T cells recovered in these wells. The incorporation of a constitutive cytokine receptor will significantly improve the long term cytotoxic function of cells when repeatedly challenged with antigen.

With respect to the use of plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those of skill within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

Any of the features of an embodiment of the first through second aspects is applicable to all aspects and embodiments identified herein. Moreover, any of the features of an embodiment of the first through third aspects is independently combinable, partly, or wholly with other embodiments described herein in any way, e.g., one, two, or three or more embodiments may be combinable in whole or in part. Further, any of the features of an embodiment of the first through third aspects may be made optional to other aspects or embodiments.

1. A protein construct capable of dimerizing, comprising:
a receptor-specific targeting region;
a transmembrane domain;
an intracellular signaling domain; and
an Fv domain comprising at least one dimerization interface,
wherein the at least one dimerization interface is capable of forming at least one disulfide bond,
wherein the protein forms a dimer upon expression,
wherein the protein construct is functional as part of a heterodimer.

2. (canceled)

3. The protein construct of claim 1, wherein the heterodimer is stabilized by at least 1 disulfide bond.

4. The protein construct of claim 1, wherein the protein construct comprises a sequence with at least 80% identity to any one of SEQ ID NOs: 1-12.

5. The protein construct of claim 4, wherein the protein construct further comprises a VH domain and a VL domain with at least 80% identity to any one of SEQ ID NOs: 83-96.

6. The protein construct of claim 1, wherein dimerization stabilizes the heavy and the light chain regions of the Fv domain.

7. The protein construct of claim 1, wherein the protein is expressed in a cell, tissue, or organ.

8. The protein construct of claim 1, wherein the protein is expressed in a mammal or human.

9. The protein construct of claim 1, wherein the receptor-specific targeting region is functionalized only upon dimerization of the protein.

10. The protein construct of claim 1, wherein the receptor-specific targeting region targets at least one of a CTLA-4 receptor, a TIM-3 receptor, a TIGIT receptor, a LAG-3 receptor, a BTLA receptor, a 2B4 receptor, a CD160 receptor, a CD226 receptor, a KIR2DL4 receptor, a CD244 receptor, a TGF-β receptor, or a PD1 receptor.

11. The protein construct of claim 9, wherein binding of the receptor-specific targeting region blocks at least one immunoinhibitory pathway.

12. The protein construct of claim 1, wherein the intracellular signaling domain is functionalized upon dimerization of the protein.

13. The protein construct of claim 12, wherein activation of the intracellular signaling domain promotes cytokine signaling.

14. The protein construct of claim 13, wherein at least 1 cytokine is upregulated upon activation of the intracellular signaling domain.

15. The protein construct of claim 1, wherein the protein construct is a chimeric antigen receptor (CAR).

16. The protein construct of claim 1, wherein the protein comprises an amino acid sequence having at least 90% sequence identity to any one of SEQ ID NOs: 1, 5, 17, or 18.

17. The protein construct of claim 1, wherein at least one disulfide bond is formed between pair(s) of cysteine residues selected from the group consisting of:
i. VH37 and VL95;
ii. VH44 and VL100;
iii. VH44 and VL105;
iv. VH45 and VL87;
v. VH55 and VL101;
vi. VH100 and VL50;
vii. VH100b and VL49;
viii. VH98 and VL46; or
ix. VH105 and VL43;
wherein the residue numbering is as determined using the Kabat numbering system.

18. (canceled)

19. (canceled)

20. A cell comprising the protein construct of claim 1.

21. The cell of claim 20, wherein the cell is a T lymphocyte or a cytotoxic T lymphocyte.

22. (canceled)

23. A method of treating a disease or disorder in a patient, the method comprising:
administering to the patient a cell expressing a protein construct, the protein construct comprising:
a receptor-specific targeting region;
a transmembrane domain;
an intracellular signaling domain; and
an Fv domain comprising at least one dimerization interface,
wherein the at least one dimerization interface is capable of forming at least one disulfide bond, and
wherein the protein construct is functional as part of a heterodimer.

24. (canceled)

25. (canceled)

26. (canceled)

27. (canceled)

28. (canceled)

29. The method of claim 23, wherein the disease or disorder is a cancer.

30. (canceled)
